SEQUENCE LISTING (60 sequences)

SEQ ID NO: 1 - protein, 15 aa, synthetic construct
MWFLTTLLLWVPVDG

SEQ ID NO: 2 - protein, 23 aa, synthetic construct
MDWIWRILFLVGAATGAHSAQPA

SEQ ID NO: 3 - DNA, 39 bp, synthetic construct
  source 1..39: mol_type = other DNA
gaaataagagagaaaagaagagtaagaagaaatataaga

SEQ ID NO: 4 - protein, 248 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSS

SEQ ID NO: 5 - protein, 239 aa, synthetic construct
VQLQQSGPELVKPGASVKMSCKASGYTFTSYVMHWVKQKPGQGLEWIGYIIPYNDATKYNEKFKGKATLTSDKSSSTAYMELSSLTSEDSAVYYCARYNYDGYFDVWGAGTTVTVSSGGGGSGGGGSGGGGSDIVMTQSPATLSVTPGDRVSLSCRASQSISDYLHWYQQKSHESPRLLIKYASQSISGIPSRFSGSGSGSDFTLSINSVEPEDVGVYYCQNGHSFPPTFGGGTKLEIK

SEQ ID NO: 6 - protein, 238 aa, synthetic construct
IVMTQSPATLSVTPGDRVSLSCRASQSISDYLHWYQQKSHESPRLLIKYASQSISGIPSRFSGSGSGSDFTLSINSVEPEDVGVYYCQNGHSFPPTFGGGTKLEIKGGGGSGGGGSGGGGSVQLQQSGPELVKPGASVKMSCKASGYTFTSYVMHWVKQKPGQGLEWIGYIIPYNDATKYNEKFKGKATLTSDKSSSTAYMELSSLTSEDSAVYYCARYNYDGYFDVWGAGTTVTVSS

SEQ ID NO: 7 - protein, 246 aa, Synthetic construct
EVQLVESGGGLVQPGGSLRLSCAASGFTFNSYAMHWVRQAPGKGLEWVSAISGNGGSTRYADSVKGRFTISRDNSKNTLYLQMNSLRAEDTAVYYCARDRFRKVHGFDVWGQGTLVTVSSGGGGSGGGGSGGGGSGGGGDIQMTQSPSSLSASVGDRVTITCQASQDISNYLNWYQQKPGKAPKLLIYDASNLETGVPSRFSGSGSGTDFTFTISSLQPEDIATYYCQQDATFPLTFGQGTKVEIK

SEQ ID NO: 8 - protein, 598 aa, synthetic construct
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 9 - protein, 39 aa, synthetic construct
IEVMYPPPYLDNEKSNGTIIHVKGKHLCPSPLFPGPSKP

SEQ ID NO: 10 - protein, 27 aa, synthetic construct
FWVLVVVGGVLACYSLLVTVAFIIFWV

SEQ ID NO: 11 - protein, 41 aa, synthetic construct
RSKRSRLLHSDYMNMTPRRPGPTRKHYQPYAPPRDFAAYRS

SEQ ID NO: 12 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 13 - DNA, 579 bp, synthetic construct
  source 1..579: mol_type = other DNA
ggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagc

SEQ ID NO: 14 - protein, 483 aa, synthetic construct
MWFLTTLLLWVPVDGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 15 - protein, 491 aa, synthetic construct
MDWIWRILFLVGAATGAHSAQPADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 16 - protein, 474 aa, synthetic construct
MWFLTTLLLWVPVDGVQLQQSGPELVKPGASVKMSCKASGYTFTSYVMHWVKQKPGQGLEWIGYIIPYNDATKYNEKFKGKATLTSDKSSSTAYMELSSLTSEDSAVYYCARYNYDGYFDVWGAGTTVTVSSGGGGSGGGGSGGGGSDIVMTQSPATLSVTPGDRVSLSCRASQSISDYLHWYQQKSHESPRLLIKYASQSISGIPSRFSGSGSGSDFTLSINSVEPEDVGVYYCQNGHSFPPTFGGGTKLEIKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 17 - protein, 474 aa, synthetic construct
MWFLTTLLLWVPVDGDIVMTQSPATLSVTPGDRVSLSCRASQSISDYLHWYQQKSHESPRLLIKYASQSISGIPSRFSGSGSGSDFTLSINSVEPEDVGVYYCQNGHSFPPTFGGGTKLEIKGGGGSGGGGSGGGGSVQLQQSGPELVKPGASVKMSCKASGYTFTSYVMHWVKQKPGQGLEWIGYIIPYNDATKYNEKFKGKATLTSDKSSSTAYMELSSLTSEDSAVYYCARYNYDGYFDVWGAGTTVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 18 - protein, 482 aa, synthetic construct
MDWIWRILFLVGAATGAHSAQPAVQLQQSGPELVKPGASVKMSCKASGYTFTSYVMHWVKQKPGQGLEWIGYIIPYNDATKYNEKFKGKATLTSDKSSSTAYMELSSLTSEDSAVYYCARYNYDGYFDVWGAGTTVTVSSGGGGSGGGGSGGGGSDIVMTQSPATLSVTPGDRVSLSCRASQSISDYLHWYQQKSHESPRLLIKYASQSISGIPSRFSGSGSGSDFTLSINSVEPEDVGVYYCQNGHSFPPTFGGGTKLEIKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 19 - protein, 482 aa, synthetic construct
MDWIWRILFLVGAATGAHSAQPADIVMTQSPATLSVTPGDRVSLSCRASQSISDYLHWYQQKSHESPRLLIKYASQSISGIPSRFSGSGSGSDFTLSINSVEPEDVGVYYCQNGHSFPPTFGGGTKLEIKGGGGSGGGGSGGGGSVQLQQSGPELVKPGASVKMSCKASGYTFTSYVMHWVKQKPGQGLEWIGYIIPYNDATKYNEKFKGKATLTSDKSSSTAYMELSSLTSEDSAVYYCARYNYDGYFDVWGAGTTVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 20 - protein, 481 aa, synthetic construct
MWFLTTLLLWVPVDGEVQLVESGGGLVQPGGSLRLSCAASGFTFNSYAMHWVRQAPGKGLEWVSAISGNGGSTRYADSVKGRFTISRDNSKNTLYLQMNSLRAEDTAVYYCARDRFRKVHGFDVWGQGTLVTVSSGGGGSGGGGSGGGGSGGGGDIQMTQSPSSLSASVGDRVTITCQASQDISNYLNWYQQKPGKAPKLLIYDASNLETGVPSRFSGSGSGTDFTFTISSLQPEDIATYYCQQDATFPLTFGQGTKVEIKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 21 - protein, 489 aa, synthetic construct
MDWIWRILFLVGAATGAHSAQPAEVQLVESGGGLVQPGGSLRLSCAASGFTFNSYAMHWVRQAPGKGLEWVSAISGNGGSTRYADSVKGRFTISRDNSKNTLYLQMNSLRAEDTAVYYCARDRFRKVHGFDVWGQGTLVTVSSGGGGSGGGGSGGGGSGGGGDIQMTQSPSSLSASVGDRVTITCQASQDISNYLNWYQQKPGKAPKLLIYDASNLETGVPSRFSGSGSGTDFTFTISSLQPEDIATYYCQQDATFPLTFGQGTKVEIKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 22 - protein, 833 aa, synthetic construct
MWFLTTLLLWVPVDGQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 23 - DNA, 45 bp, synthetic construct
  source 1..45: mol_type = other DNA
atgtggtttcttactacattgctgttgtgggtcccggtggacggt

SEQ ID NO: 24 - DNA, 1449 bp, synthetic construct
  source 1..1449: mol_type = other DNA
atgtggtttcttactacattgctgttgtgggtcccggtggacggtgatatccagatgacccagacaacaagcagcctgagcgcctctctgggcgatagagtgacaatcagctgcagagccagccaggacatcagcaagtacctgaactggtatcagcagaaacccgacggcaccgtgaagctgctgatctaccacacaagcagactgcacagcggcgtgccaagcagattttctggcagcggcagcggcaccgattacagcctgaccatcagcaacctggaacaggaagatatcgctacctacttctgtcagcagggcaacaccctgccttacacctttggcggcggaacaaagctggaactgaaaagaggcggcggaggaagcggaggcggaggatctgggggcggaggctctggcggagggggatctgaagtgcagctgcagcagtctggacctggactggtggctccttctcagtccctgtctgtgacctgtacagtgtctggcgtgtccctgcctgattacggcgtgtcctggatcagacagcctcccagaaaaggcctggaatggctgggagtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggattattggggccagggcaccaccgtgacagtgtcatctattgaagttatgtatcctcctccttacctagacaatgagaagagtaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 25 - DNA, 1473 bp, synthetic construct
  source 1..1473: mol_type = other DNA
atggactggatctggcggatcctgtttctcgtgggagctgccacaggcgctcattctgctcagcctgccgatatccagatgacccagacaacaagcagcctgagcgcctctctgggcgatagagtgacaatcagctgcagagccagccaggacatcagcaagtacctgaactggtatcagcagaaacccgacggcaccgtgaagctgctgatctaccacacaagcagactgcacagcggcgtgccaagcagattttctggcagcggcagcggcaccgattacagcctgaccatcagcaacctggaacaggaagatatcgctacctacttctgtcagcagggcaacaccctgccttacacctttggcggcggaacaaagctggaactgaaaagaggcggcggaggaagcggaggcggaggatctgggggcggaggctctggcggagggggatctgaagtgcagctgcagcagtctggacctggactggtggctccttctcagtccctgtctgtgacctgtacagtgtctggcgtgtccctgcctgattacggcgtgtcctggatcagacagcctcccagaaaaggcctggaatggctgggagtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggattattggggccagggcaccaccgtgacagtgtcatctattgaagttatgtatcctcctccttacctagacaatgagaagagtaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 26 - DNA, 1422 bp, synthetic construct
  source 1..1422: mol_type = other DNA
atgtggtttcttactacattgctgttgtgggtcccggtggacggtgtccagctgcagcagtctggacctgagctggtaaagcctggggcttcagtgaagatgtcctgcaaggcttctggatacacattcactagctatgttatgcactgggtgaagcagaagcctgggcagggccttgagtggattggatatattattccttacaatgatgctactaagtacaatgagaagttcaaaggcaaggccacactgacttcagacaaatcctccagcacagcctacatggagctcagcagcctgacctctgaggactctgcggtctattactgtgcacgctataattacgacgggtacttcgatgtctggggcgcagggaccacggtcaccgtctcctcaggcggcggaggaagcggaggcggaggatctgggggcggaggctctgacattgtgatgactcagtctccagccaccctgtctgtgactccaggagatagagtctctctttcctgcagggccagccagagtattagcgactacttacactggtatcaacaaaaatcacatgagtctccaaggcttctcatcaaatatgcttcccaatccatctctggaatcccctccaggttcagtggcagtggatcagggtcagatttcactctcagtatcaacagtgtggaacctgaagatgttggagtgtattactgtcaaaatggtcacagctttcctccgacgttcggtggaggcaccaagctggaaatcaaaattgaagttatgtatcctcctccttacctagacaatgagaagagtaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 27 - DNA, 1422 bp, synthetic construct
  source 1..1422: mol_type = other DNA
atgtggtttcttactacattgctgttgtgggtcccggtggacggtgacattgtgatgactcagtctccagccaccctgtctgtgactccaggagatagagtctctctttcctgcagggccagccagagtattagcgactacttacactggtatcaacaaaaatcacatgagtctccaaggcttctcatcaaatatgcttcccaatccatctctggaatcccctccaggttcagtggcagtggatcagggtcagatttcactctcagtatcaacagtgtggaacctgaagatgttggagtgtattactgtcaaaatggtcacagctttcctccgacgttcggtggaggcaccaagctggaaatcaaaggcggcggaggaagcggaggcggaggatctgggggcggaggctctgtccagctgcagcagtctggacctgagctggtaaagcctggggcttcagtgaagatgtcctgcaaggcttctggatacacattcactagctatgttatgcactgggtgaagcagaagcctgggcagggccttgagtggattggatatattattccttacaatgatgctactaagtacaatgagaagttcaaaggcaaggccacactgacttcagacaaatcctccagcacagcctacatggagctcagcagcctgacctctgaggactctgcggtctattactgtgcacgctataattacgacgggtacttcgatgtctggggcgcagggaccacggtcaccgtctcctcaattgaagttatgtatcctcctccttacctagacaatgagaagagtaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 28 - DNA, 1446 bp, synthetic construct
  source 1..1446: mol_type = other DNA
atggactggatctggcggattctgtttctcgtgggagctgccacaggcgctcattctgctcagcctgccgtccagctgcagcagtctggacctgagctggtaaagcctggggcttcagtgaagatgtcctgcaaggcttctggatacacattcactagctatgttatgcactgggtgaagcagaagcctgggcagggccttgagtggattggatatattattccttacaatgatgctactaagtacaatgagaagttcaaaggcaaggccacactgacttcagacaaatcctccagcacagcctacatggagctcagcagcctgacctctgaggactctgcggtctattactgtgcacgctataattacgacgggtacttcgatgtctggggcgcagggaccacggtcaccgtctcctcaggcggcggaggaagcggaggcggaggatctgggggcggaggctctgacattgtgatgactcagtctccagccaccctgtctgtgactccaggagatagagtctctctttcctgcagggccagccagagtattagcgactacttacactggtatcaacaaaaatcacatgagtctccaaggcttctcatcaaatatgcttcccaatccatctctggaatcccctccaggttcagtggcagtggatcagggtcagatttcactctcagtatcaacagtgtggaacctgaagatgttggagtgtattactgtcaaaatggtcacagctttcctccgacgttcggtggaggcaccaagctggaaatcaaaattgaagttatgtatcctcctccttacctagacaatgagaagagtaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 29 - DNA, 1446 bp, synthetic construct
  source 1..1446: mol_type = other DNA
atggactggatctggcggattctgtttctcgtgggagctgccacaggcgctcattctgctcagcctgccgacattgtgatgactcagtctccagccaccctgtctgtgactccaggagatagagtctctctttcctgcagggccagccagagtattagcgactacttacactggtatcaacaaaaatcacatgagtctccaaggcttctcatcaaatatgcttcccaatccatctctggaatcccctccaggttcagtggcagtggatcagggtcagatttcactctcagtatcaacagtgtggaacctgaagatgttggagtgtattactgtcaaaatggtcacagctttcctccgacgttcggtggaggcaccaagctggaaatcaaaggcggcggaggaagcggaggcggaggatctgggggcggaggctctgtccagctgcagcagtctggacctgagctggtaaagcctggggcttcagtgaagatgtcctgcaaggcttctggatacacattcactagctatgttatgcactgggtgaagcagaagcctgggcagggccttgagtggattggatatattattccttacaatgatgctactaagtacaatgagaagttcaaaggcaaggccacactgacttcagacaaatcctccagcacagcctacatggagctcagcagcctgacctctgaggactctgcggtctattactgtgcacgctataattacgacgggtacttcgatgtctggggcgcagggaccacggtcaccgtctcctcaattgaagttatgtatcctcctccttacctagacaatgagaagagtaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 30 - DNA, 1443 bp, synthetic construct
  source 1..1443: mol_type = other DNA
atgtggtttcttactacattgctgttgtgggtcccggtggacggtgaagttcagcttgtagaatctggaggtggattggttcaacctggtggctctcttcgcctgagttgtgcagcctctggttttactttcaatagttacgctatgcattgggttcgtcaggctcctgggaaaggcctggaatgggtttcagctattagtggtaatggaggtagtactcgttacgcagacagtgtgaaaggtcgcttcaccatcagccgtgataattctaagaacactttgtacctgcaaatgaactccttgcgcgcagaagacacggctgtgtactattgtgcccgtgatcgctttcggaaggttcatggtttcgatgtatggggacaaggtactctggtaacggtttctagcggaggtggtgggagtggtggaggcggctcgggtggaggtggttcaggaggaggcggagatatccaaatgactcaatctcctagttcactgtcagcctctgttggtgatcgcgtgaccattacctgccaagctagccaggatattagcaactacttgaactggtatcagcagaagcctggcaaagccccaaagctgttgatctacgatgcaagtaacttggaaactggcgtcccaagccgcttctctggatctggttcaggcaccgacttcactttcactatcagcagcctgcagcctgaagatatcgcaacctactattgccagcaggatgctacttttcctttgactttcggccaaggcaccaaggtggagatcaagattgaagttatgtatcctcctccttacctagacaatgagaagagtaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 31 - DNA, 1467 bp, synthetic construct
  source 1..1467: mol_type = other DNA
atggactggatctggcggattctgtttctcgtgggagctgccacaggcgctcattctgctcagcctgccgaagttcagcttgtagaatctggaggtggattggttcaacctggtggctctcttcgcctgagttgtgcagcctctggttttactttcaatagttacgctatgcattgggttcgtcaggctcctgggaaaggcctggaatgggtttcagctattagtggtaatggaggtagtactcgttacgcagacagtgtgaaaggtcgcttcaccatcagccgtgataattctaagaacactttgtacctgcaaatgaactccttgcgcgcagaagacacggctgtgtactattgtgcccgtgatcgctttcggaaggttcatggtttcgatgtatggggacaaggtactctggtaacggtttctagcggaggtggtgggagtggtggaggcggctcgggtggaggtggttcaggaggaggcggagatatccaaatgactcaatctcctagttcactgtcagcctctgttggtgatcgcgtgaccattacctgccaagctagccaggatattagcaactacttgaactggtatcagcagaagcctggcaaagccccaaagctgttgatctacgatgcaagtaacttggaaactggcgtcccaagccgcttctctggatctggttcaggcaccgacttcactttcactatcagcagcctgcagcctgaagatatcgcaacctactattgccagcaggatgctacttttcctttgactttcggccaaggcaccaaggtggagatcaagattgaagttatgtatcctcctccttacctagacaatgagaagagtaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 32 - DNA, 2499 bp, synthetic construct
  source 1..2499: mol_type = other DNA
atgtggtttcttactacattgctgttgtgggtcccggtggacggtcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaaacttcagctgcaggctctacagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacattgaagttatgtatcctcctccttacctagacaatgagaagagtaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 33 - DNA, 8764 bp, synthetic construct
  source 1..8764: mol_type = other DNA
actagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagctttaatacgactcactatagggaaataagagagaaaagaagagtaagaagaaatataagagccaccatgtggtttcttactacattgctgttgtgggtcccggtggacggtcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaaacttcagctgcaggctctacagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagacattgaagttatgtatcctcctccttacctagacaatgagaagagtaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgattaataagctgccttctgcggggcttgccttctggccatgcccttcttctctcccttgcacctgtacctcttggtctttgaataaagcctgagtaggaagggtaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagctctaggctagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctatcctcatgcatatacagtcagcatatgatacccagtagtagagtgggagtgctatcctttgcatagcccatcccgcccctaactccgcccagttccgcccattctccgcctcatggctgactaattttttaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcata

SEQ ID NO: 34 - DNA, 7682 bp, synthetic construct
  source 1..7682: mol_type = other DNA
actagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagctttaatacgactcactatagggaaataagagagaaaagaagagtaagaagaaatataagagccaccatgtggtttcttactacattgctgttgtgggtcccggtggacggtgaagttcagcttgtagaatctggaggtggattggttcaacctggtggctctcttcgcctgagttgtgcagcctctggttttactttcaatagttacgctatgcattgggttcgtcaggctcctgggaaaggcctggaatgggtttcagctattagtggtaatggaggtagtactcgttacgcagacagtgtgaaaggtcgcttcaccatcagccgtgataattctaagaacactttgtacctgcaaatgaactccttgcgcgcagaagacacggctgtgtactattgtgcccgtgatcgctttcggaaggttcatggtttcgatgtatggggacaaggtactctggtaacggtttctagcggaggtggtgggagtggtggaggcggctcgggtggaggtggttcaggaggaggcggagatatccaaatgactcaatctcctagttcactgtcagcctctgttggtgatcgcgtgaccattacctgccaagctagccaggatattagcaactacttgaactggtatcagcagaagcctggcaaagccccaaagctgttgatctacgatgcaagtaacttggaaactggcgtcccaagccgcttctctggatctggttcaggcaccgacttcactttcactatcagcagcctgcagcctgaagatatcgcaacctactattgccagcaggatgctacttttcctttgactttcggccaaggcaccaaggtggagatcaagattgaagttatgtatcctcctccttacctagacaatgagaagagtaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgattaattaagctgccttctgcggggcttgccttctggccatgcccttcttctctcccttgcacctgtacctcttggtctttgaataaagcctgagtaggaagggtaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagctctaggctagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctatcctcatgcatatacagtcagcatatgatacccagtagtagagtgggagtgctatcctttgcatagcccatcccgcccctaactccgcccagttccgcccattctccgcctcatggctgactaattttttaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggc

SEQ ID NO: 35 - DNA, 7713 bp, synthetic construct
  source 1..7713: mol_type = other DNA
actagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagctttaatacgactcactatagggagacccaagctggaattcgccaccatggactggatctggcggattctgtttctcgtgggagctgccacaggcgctcattctgctcagcctgccgaagttcagcttgtagaatctggaggtggattggttcaacctggtggctctcttcgcctgagttgtgcagcctctggttttactttcaatagttacgctatgcattgggttcgtcaggctcctgggaaaggcctggaatgggtttcagctattagtggtaatggaggtagtactcgttacgcagacagtgtgaaaggtcgcttcaccatcagccgtgataattctaagaacactttgtacctgcaaatgaactccttgcgcgcagaagacacggctgtgtactattgtgcccgtgatcgctttcggaaggttcatggtttcgatgtatggggacaaggtactctggtaacggtttctagcggaggtggtgggagtggtggaggcggctcgggtggaggtggttcaggaggaggcggagatatccaaatgactcaatctcctagttcactgtcagcctctgttggtgatcgcgtgaccattacctgccaagctagccaggatattagcaactacttgaactggtatcagcagaagcctggcaaagccccaaagctgttgatctacgatgcaagtaacttggaaactggcgtcccaagccgcttctctggatctggttcaggcaccgacttcactttcactatcagcagcctgcagcctgaagatatcgcaacctactattgccagcaggatgctacttttcctttgactttcggccaaggcaccaaggtggagatcaagattgaagttatgtatcctcctccttacctagacaatgagaagagtaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgattaattaagctgccttctgcggggcttgccttctggccatgcccttcttctctcccttgcacctgtacctcttggtctttgaataaagcctgagtaggaagggtaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagctctaggctagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctatcctcatgcatatacagtcagcatatgatacccagtagtagagtgggagtgctatcctttgcatagcccatcccgcccctaactccgcccagttccgcccattctccgcctcatggctgactaattttttaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcata

SEQ ID NO: 36 - DNA, 7642 bp, synthetic construct
  source 1..7642: mol_type = other DNA
actagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagctttaatacgactcactatagggagacccaagctggaattcgccaccatggactggatctggcggattctgtttctcgtgggagctgccacaggcgctcattctgctcagcctgccgtccagctgcagcagtctggacctgagctggtaaagcctggggcttcagtgaagatgtcctgcaaggcttctggatacacattcactagctatgttatgcactgggtgaagcagaagcctgggcagggccttgagtggattggatatattattccttacaatgatgctactaagtacaatgagaagttcaaaggcaaggccacactgacttcagacaaatcctccagcacagcctacatggagctcagcagcctgacctctgaggactctgcggtctattactgtgcacgctataattacgacgggtacttcgatgtctggggcgcagggaccacggtcaccgtctcctcaggcggcggaggaagcggaggcggaggatctgggggcggaggctctgacattgtgatgactcagtctccagccaccctgtctgtgactccaggagatagagtctctctttcctgcagggccagccagagtattagcgactacttacactggtatcaacaaaaatcacatgagtctccaaggcttctcatcaaatatgcttcccaatccatctctggaatcccctccaggttcagtggcagtggatcagggtcagatttcactctcagtatcaacagtgtggaacctgaagatgttggagtgtattactgtcaaaatggtcacagctttcctccgacgttcggtggaggcaccaagctggaaatcaaaattgaagttatgtatcctcctccttacctagacaatgagaagagtaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgattaattaagctgccttctgcggggcttgccttctggccatgcccttcttctctcccttgcacctgtacctcttggtctttgaataaagcctgagtaggaagggtaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagctctaggctagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctatcctcatgcatatacagtcagcatatgatacccagtagtagagtgggagtgctatcctttgcatagcccatcccgcccctaactccgcccagttccgcccattctccgcctcatggctgactaattttttaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttt

SEQ ID NO: 37 - DNA, 7688 bp, synthetic construct
  source 1..7688: mol_type = other DNA
actagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagctttaatacgactcactatagggaaataagagagaaaagaagagtaagaagaaatataagagccaccatgtggtttcttactacattgctgttgtgggtcccggtggacggtgtccagctgcagcagtctggacctgagctggtaaagcctggggcttcagtgaagatgtcctgcaaggcttctggatacacattcactagctatgttatgcactgggtgaagcagaagcctgggcagggccttgagtggattggatatattattccttacaatgatgctactaagtacaatgagaagttcaaaggcaaggccacactgacttcagacaaatcctccagcacagcctacatggagctcagcagcctgacctctgaggactctgcggtctattactgtgcacgctataattacgacgggtacttcgatgtctggggcgcagggaccacggtcaccgtctcctcaggcggcggaggaagcggaggcggaggatctgggggcggaggctctgacattgtgatgactcagtctccagccaccctgtctgtgactccaggagatagagtctctctttcctgcagggccagccagagtattagcgactacttacactggtatcaacaaaaatcacatgagtctccaaggcttctcatcaaatatgcttcccaatccatctctggaatcccctccaggttcagtggcagtggatcagggtcagatttcactctcagtatcaacagtgtggaacctgaagatgttggagtgtattactgtcaaaatggtcacagctttcctccgacgttcggtggaggcaccaagctggaaatcaaaattgaagttatgtatcctcctccttacctagacaatgagaagagtaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgattaattaagctgccttctgcggggcttgccttctggccatgcccttcttctctcccttgcacctgtacctcttggtctttgaataaagcctgagtaggaagggtaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagctctaggctagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctatcctcatgcatatacagtcagcatatgatacccagtagtagagtgggagtgctatcctttgcatagcccatcccgcccctaactccgcccagttccgcccattctccgcctcatggctgactaattttttaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcata

SEQ ID NO: 38 - DNA, 7692 bp, synthetic construct
  source 1..7692: mol_type = other DNA
actagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagctttaatacgactcactatagggagacccaagctggaattcgccaccatggactggatctggcggattctgtttctcgtgggagctgccacaggcgctcattctgctcagcctgccgacattgtgatgactcagtctccagccaccctgtctgtgactccaggagatagagtctctctttcctgcagggccagccagagtattagcgactacttacactggtatcaacaaaaatcacatgagtctccaaggcttctcatcaaatatgcttcccaatccatctctggaatcccctccaggttcagtggcagtggatcagggtcagatttcactctcagtatcaacagtgtggaacctgaagatgttggagtgtattactgtcaaaatggtcacagctttcctccgacgttcggtggaggcaccaagctggaaatcaaaggcggcggaggaagcggaggcggaggatctgggggcggaggctctgtccagctgcagcagtctggacctgagctggtaaagcctggggcttcagtgaagatgtcctgcaaggcttctggatacacattcactagctatgttatgcactgggtgaagcagaagcctgggcagggccttgagtggattggatatattattccttacaatgatgctactaagtacaatgagaagttcaaaggcaaggccacactgacttcagacaaatcctccagcacagcctacatggagctcagcagcctgacctctgaggactctgcggtctattactgtgcacgctataattacgacgggtacttcgatgtctggggcgcagggaccacggtcaccgtctcctcaattgaagttatgtatcctcctccttacctagacaatgagaagagtaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgattaattaagctgccttctgcggggcttgccttctggccatgcccttcttctctcccttgcacctgtacctcttggtctttgaataaagcctgagtaggaagggtaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagctctaggctagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctatcctcatgcatatacagtcagcatatgatacccagtagtagagtgggagtgctatcctttgcatagcccatcccgcccctaactccgcccagttccgcccattctccgcctcatggctgactaattttttaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcata

SEQ ID NO: 39 - DNA, 7688 bp, synthetic construct
  source 1..7688: mol_type = other DNA
actagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagctttaatacgactcactatagggaaataagagagaaaagaagagtaagaagaaatataagagccaccatgtggtttcttactacattgctgttgtgggtcccggtggacggtgacattgtgatgactcagtctccagccaccctgtctgtgactccaggagatagagtctctctttcctgcagggccagccagagtattagcgactacttacactggtatcaacaaaaatcacatgagtctccaaggcttctcatcaaatatgcttcccaatccatctctggaatcccctccaggttcagtggcagtggatcagggtcagatttcactctcagtatcaacagtgtggaacctgaagatgttggagtgtattactgtcaaaatggtcacagctttcctccgacgttcggtggaggcaccaagctggaaatcaaaggcggcggaggaagcggaggcggaggatctgggggcggaggctctgtccagctgcagcagtctggacctgagctggtaaagcctggggcttcagtgaagatgtcctgcaaggcttctggatacacattcactagctatgttatgcactgggtgaagcagaagcctgggcagggccttgagtggattggatatattattccttacaatgatgctactaagtacaatgagaagttcaaaggcaaggccacactgacttcagacaaatcctccagcacagcctacatggagctcagcagcctgacctctgaggactctgcggtctattactgtgcacgctataattacgacgggtacttcgatgtctggggcgcagggaccacggtcaccgtctcctcaattgaagttatgtatcctcctccttacctagacaatgagaagagtaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgattaattaagctgccttctgcggggcttgccttctggccatgcccttcttctctcccttgcacctgtacctcttggtctttgaataaagcctgagtaggaagggtaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagctctaggctagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctatcctcatgcatatacagtcagcatatgatacccagtagtagagtgggagtgctatcctttgcatagcccatcccgcccctaactccgcccagttccgcccattctccgcctcatggctgactaattttttaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcata

SEQ ID NO: 40 - DNA, 7715 bp, synthetic construct
  source 1..7715: mol_type = other DNA
actagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagctttaatacgactcactatagggaaataagagagaaaagaagagtaagaagaaatataagagccaccatgtggtttcttactacattgctgttgtgggtcccggtggacggtgatatccagatgacccagacaacaagcagcctgagcgcctctctgggcgatagagtgacaatcagctgcagagccagccaggacatcagcaagtacctgaactggtatcagcagaaacccgacggcaccgtgaagctgctgatctaccacacaagcagactgcacagcggcgtgccaagcagattttctggcagcggcagcggcaccgattacagcctgaccatcagcaacctggaacaggaagatatcgctacctacttctgtcagcagggcaacaccctgccttacacctttggcggcggaacaaagctggaactgaaaagaggcggcggaggaagcggaggcggaggatctgggggcggaggctctggcggagggggatctgaagtgcagctgcagcagtctggacctggactggtggctccttctcagtccctgtctgtgacctgtacagtgtctggcgtgtccctgcctgattacggcgtgtcctggatcagacagcctcccagaaaaggcctggaatggctgggagtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggattattggggccagggcaccaccgtgacagtgtcatctattgaagttatgtatcctcctccttacctagacaatgagaagagtaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgattaattaagctgccttctgcggggcttgccttctggccatgcccttcttctctcccttgcacctgtacctcttggtctttgaataaagcctgagtaggaagggtaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagctctaggctagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctatcctcatgcatatacagtcagcatatgatacccagtagtagagtgggagtgctatcctttgcatagcccatcccgcccctaactccgcccagttccgcccattctccgcctcatggctgactaattttttaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcata

SEQ ID NO: 41 - DNA, 7739 bp, synthetic construct
  source 1..7739: mol_type = other DNA
actagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagctttaatacgactcactatagggaaataagagagaaaagaagagtaagaagaaatataagagccaccatggactggatctggcggatcctgtttctcgtgggagctgccacaggcgctcattctgctcagcctgccgatatccagatgacccagacaacaagcagcctgagcgcctctctgggcgatagagtgacaatcagctgcagagccagccaggacatcagcaagtacctgaactggtatcagcagaaacccgacggcaccgtgaagctgctgatctaccacacaagcagactgcacagcggcgtgccaagcagattttctggcagcggcagcggcaccgattacagcctgaccatcagcaacctggaacaggaagatatcgctacctacttctgtcagcagggcaacaccctgccttacacctttggcggcggaacaaagctggaactgaaaagaggcggcggaggaagcggaggcggaggatctgggggcggaggctctggcggagggggatctgaagtgcagctgcagcagtctggacctggactggtggctccttctcagtccctgtctgtgacctgtacagtgtctggcgtgtccctgcctgattacggcgtgtcctggatcagacagcctcccagaaaaggcctggaatggctgggagtgatctggggcagcgagacaacctactacaacagcgccctgaagtcccggctgaccatcatcaaggacaacagcaagagccaggtgttcctgaagatgaacagcctgcagaccgacgacaccgccatctactactgcgccaagcactactactacggcggcagctacgccatggattattggggccagggcaccaccgtgacagtgtcatctattgaagttatgtatcctcctccttacctagacaatgagaagagtaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgattaattaagctgccttctgcggggcttgccttctggccatgcccttcttctctcccttgcacctgtacctcttggtctttgaataaagcctgagtaggaagggtaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagctctaggctagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctatcctcatgcatatacagtcagcatatgatacccagtagtagagtgggagtgctatcctttgcatagcccatcccgcccctaactccgcccagttccgcccattctccgcctcatggctgactaattttttaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcata

SEQ ID NO: 42 - DNA, 7739 bp, synthetic construct
  source 1..7739: mol_type = other DNA
actagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagctttaatacgactcactatagggaaataagagagaaaagaagagtaagaagaaatataagagccaccatgtggtttcttactacattgctgttgtgggtcccggtggacggtgatatccaaatgactcaatctcctagttcactgtcagcctctgttggtgatcgcgtgaccattacctgccaagctagccaggatattagcaactacttgaactggtatcagcagaagcctggcaaagccccaaagctgttgatctacgatgtaagtaacttggaaactggcgtcccaagccgcttctctggatctggttcaggcaccgacttcactttcactatcagcagcctgcagcctgaagatatcgcaacctactattgccagcaggttgctaatgttcctctgactttcggccaaggcaccaaggtggagatcaagtcctcagggggcgggggaagtggtgggggcggcagcggcggagggggctcaggaggaggcggatcaggcggatcagaagttcagcttgtagaatctggaggtggattggttcaacctggtggctctcttcgcctgagttgtgcagcctctggttttactttctctagttactggatgcattgggttcgtcaggctcctgggaaaggcctggaatgggtttcagctattagttggagtggagatagtacttactacgcagacagtgtgaaaggtcgcttcaccatcagccgtgataattctaagaacactttgtacctgcaaatgaactccttgcgcgcagaagacacggctgtgtactattgtgcccgtgatcgctctgcgacttggtattatctggggcttggtttcgatgtatggggacaaggtaccctggtaacggtttctagcattgaagttatgtatcctcctccttacctagacaatgagaagagtaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgattaattaagctgccttctgcggggcttgccttctggccatgcccttcttctctcccttgcacctgtacctcttggtctttgaataaagcctgagtaggaagggtaccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagctctaggctagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctatcctcatgcatatacagtcagcatatgatacccagtagtagagtgggagtgctatcctttgcatagcccatcccgcccctaactccgcccagttccgcccattctccgcctcatggctgactaattttttaggaggcttttttggaggcctaggcttttgcaaaaagctaattcggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcata

SEQ ID NO: 43 - DNA, 1182 bp, synthetic construct
  source 1..1182: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 44 - DNA, 19 bp, synthetic construct
  source 1..19: mol_type = other DNA
taatacgactcactatagg

SEQ ID NO: 45 - skipped

SEQ ID NO: 46 - DNA, 69 bp, synthetic construct
  source 1..69: mol_type = other DNA
atggactggatctggcggattctgtttctcgtgggagctgccacaggcgctcattctgctcagcctgcc

SEQ ID NO: 47 - DNA, 117 bp, synthetic construct
  source 1..117: mol_type = other DNA
attgaagttatgtatcctcctccttacctagacaatgagaagagtaatggaaccattatccatgtgaaagggaaacacctttgtccaagtcccctatttcccggaccttctaagccc

SEQ ID NO: 48 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 49 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 50 - DNA, 342 bp, synthetic construct
  source 1..342: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 51 - DNA, 94 bp, synthetic construct
  source 1..94: mol_type = other DNA
agctgccttctgcggggcttgccttctggccatgcccttcttctctcccttgcacctgtacctcttggtctttgaataaagcctgagtaggaag

SEQ ID NO: 52 - DNA, 150 bp, synthetic construct
  source 1..150: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 53 - DNA, 135 bp, synthetic construct
  source 1..135: mol_type = other DNA
aacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 54 - DNA, 600 bp, synthetic construct
  source 1..600: mol_type = other DNA
tcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcat

SEQ ID NO: 55 - DNA, 861 bp, synthetic construct
  source 1..861: mol_type = other DNA
atgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaa

SEQ ID NO: 56 - DNA, 133 bp, synthetic construct
  source 1..133: mol_type = other DNA
ggataaatttctggtaaggaggacacgtatggaagtgggcaagttggggaagccgtatccgttgctgaatctggcatatgtgggagtataagacgcgcagcgtcgcatcaggcatttttttctgcgccaatgc

SEQ ID NO: 57 - protein, 256 aa, synthetic construct
DIQMTQSPSSLSASVGDRVTITCQASQDISNYLNWYQQKPGKAPKLLIYDVSNLETGVPSRFSGSGSGTDFTFTISSLQPEDIATYYCQQVANVPLTFGQGTKVEIKSSGGGGSGGGGSGGGGSGGGGSGGSEVQLVESGGGLVQPGGSLRLSCAASGFTFSSYWMHWVRQAPGKGLEWVSAISWSGDSTYYADSVKGRFTISRDNSKNTLYLQMNSLRAEDTAVYYCARDRSATWYYLGLGFDVWGQGTLVTVSS

SEQ ID NO: 58 - protein, 491 aa, synthetic construct
MWFLTTLLLWVPVDGDIQMTQSPSSLSASVGDRVTITCQASQDISNYLNWYQQKPGKAPKLLIYDVSNLETGVPSRFSGSGSGTDFTFTISSLQPEDIATYYCQQVANVPLTFGQGTKVEIKSSGGGGSGGGGSGGGGSGGGGSGGSEVQLVESGGGLVQPGGSLRLSCAASGFTFSSYWMHWVRQAPGKGLEWVSAISWSGDSTYYADSVKGRFTISRDNSKNTLYLQMNSLRAEDTAVYYCARDRSATWYYLGLGFDVWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 59 - DNA, 1476 bp, synthetic construct
  source 1..1476: mol_type = other DNA
atgtggtttcttactacattgctgttgtgggtcccggtggacggtgatatccaaatgactcaatctcctagttcactgtcagcctctgttggtgatcgcgtgaccattacctgccaagctagccaggatattagcaactacttgaactggtatcagcagaagcctggcaaagccccaaagctgttgatctacgatgtaagtaacttggaaactggcgtcccaagccgcttctctggatctggttcaggcaccgacttcactttcactatcagcagcctgcagcctgaagatatcgcaacctactattgccagcaggttgctaatgttcctctgactttcggccaaggcaccaaggtggagatcaagtcctcagggggcgggggaagtggtgggggcggcagcggcggagggggctcaggaggaggcggatcaggcggatcagaagttcagcttgtagaatctggaggtggattggttcaacctggtggctctcttcgcctgagttgtgcagcctctggttttactttctctagttactggatgcattgggttcgtcaggctcctgggaaaggcctggaatgggtttcagctattagttggagtggagatagtacttactacgcagacagtgtgaaaggtcgcttcaccatcagccgtgataattctaagaacactttgtacctgcaaatgaactccttgcgcgcagaagacacggctgtgtactattgtgcccgtgatcgctctgcgacttggtattatctggggcttggtttcgatgtatggggacaaggtaccctggtaacggtttctagcattgaagttatgtatcctcctccttacctagacaatgagaagagtaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 60 - DNA, 19 bp, synthetic construct
  source 1..19: mol_type = other DNA
gagacccaagctggaattc

(19) United States
(12) Patent Application Publication
(10) Pub. No.: US 2025/0084175 A1
(43) Pub. Date: Mar. 13, 2025

(54) GENERATION OF CHIMERIC ANTIGEN RECEPTOR mRNA MOLECULES FOR EXPRESSION IN PRIMARY NK CELLS

(71) Applicant: IMMUNITYBIO, INC., Culver City, CA (US)

(72) Inventor: Fereshteh PARVIZ, Culver City, CA (US)

(21) Appl. No.: 18/580,025

(22) PCT Filed: Jul. 14, 2022
(86) PCT No.: PCT/US2022/073727

§ 371 Date: Jan. 17, 2024

Related U.S. Application Data
(60) Provisional application No. 63/224,100, filed on Jul. 21, 2021.

(51) Int. Cl. C07K 16/28; A61K 39/00; C07K 14/705; C07K 14/735; C07K 16/10; C12N 5/0783

(52) U.S. Cl. CPC C07K 16/2878; A61K 40/15; A61K 40/31; A61K 40/4202; A61K 40/421; A61K 40/4211; A61K 40/4224; A61K 40/46; C07K 14/70521; C07K 14/70535; C07K 16/1003; C07K 16/2803; C07K 16/2809; C07K 16/2827; C12N 5/0646; C07K 2317/53; C07K 2319/02; C07K 2319/03; C12N 2510/00

(57) ABSTRACT

The invention relates to novel chimeric antigen (CAR) mRNA molecules, the methods of generating the molecules and methods of treating cancer with the molecules.

### CROSS REFERENCE TO RELATED APPLICATIONS

This application claims the benefit of priority under 35 U.S.C. § 119 (e) to U.S. Provisional Patent Application No. 63/224,100, filed Jul. 21, 2021. The entire disclosure of U.S. Provisional Patent Application No. 63/224, 100 is incorporated herein by reference.

### REFERENCE TO A SEQUENCE LISTING

This application contains a Sequence Listing submitted electronically as a text file by EFS-Web. The XML file, named “8774-19-PCT”, has a size in bytes of 154000 bytes, and was recorded on 11 Jul. 2021. The information contained in the XML file is incorporated herein by reference in its entirety pursuant to 37 CFR § 1.52 (e) (5).

### BACKGROUND

Natural killer (abbreviated NK) cells are the key mediators of the innate immune system. NK cells can rapidly discover and destroy abnormal cells (such as cancer cells or virus-infected cells) without requiring prior sensitization or HLA matching. Using immune cells (including NK cells) to treat cancer is a new trend in recent years. This new therapy is expected to be promising for the treatment of tumors that are refractory to traditional surgery, chemotherapy and radiotherapy.

Chimeric antigen receptors (CAR) are engineered proteins composed of an extracellular receptor region fused to an intracellular signaling region. Normally these regions are from different proteins, however they can also be designed de novo. CAR expressing T cells have utilized single-chain variable fragments (scFV) fused to intracellular signaling domains, normally the zeta chain of CD3 (CD3ζ). Further developments have included secondary co-stimulatory signals, such as CD28 and CD137, to enhance T cell activation. CAR constructs have also been applied to NK cells, most notably in the use of NK-92 CD19-CAR expression for treatment of CD19+ B cell tumors, which have also been treated with T cell CD19-CARs.

The inventors have found that while many CAR molecules can be expressed in NK cell lines, they cannot be expressed as either DNA or RNA in primary NK cells. Most CAR technology uses viral vectors for the delivery of a DNA molecule into the cells. The viral DNA enters the nucleus and can integrate into the host genome with a preference for the transcriptionally active sites. As disclosed herein, the inventors have determined a novel combination of specific domains and stabilizing elements for high expression of CAR RNA molecules in NK cells.

### SUMMARY

Disclosed herein is a chimeric antigen receptor (CAR) comprising a T7 promoter, a spacer sequence, a signal peptide, an antigen binding domain, a hinge region, a transmembrane (TM) domain and an intracellular domain; wherein the signal peptide comprises a cluster of differentiation 64 (CD64) and/or an IgG heavy chain variable gene (IgGHv) signal peptide; and wherein the antigen binding domain binds to an antigen selected from the group consisting of cluster of differentiation 19 (CD19), B-cell maturation antigen (BCMA), B7 homolog 4 (B7H4), severe acute respiratory syndrome coronavirus 2 (SARS-CoV-2) spike and cluster of differentiation 30 alpha (CD30α).

In one aspect, the signal peptide comprises SEQ ID NO:1 or SEQ ID NO:2.

In one aspect, the antigen binding domain that binds to CD19 comprises at least 90%, at least 95%, or up to 100% sequence identity to SEQ ID NO:4.

In still another aspect, the antigen binding domain that binds to BCMA comprises at least 90%, at least 95%, or up to 100% sequence identity to SEQ ID NO:5 or SEQ ID NO: 6.

In yet another aspect, the antigen binding domain that binds to B7H4 comprises at least 90%, at least 95%, or up to 100% sequence identity to SEQ ID NO:7.

In one aspect, the antigen binding domain that binds to SARS-CoV-2 spike comprises at least 90%, at least 95%, or up to 100% sequence identity to SEQ ID NO:8.

In one aspect, the antigen binding domain that binds to CD30α spike comprises at least 90%, at least 95%, or up to 100% sequence identity to SEQ ID NO:57.

In one aspect, the hinge region is a cluster of differentiation 28 (CD28) hinge region having SEQ ID NO:9.

In one aspect, the TM domain is a CD28 TM domain having SEQ ID NO:10.

In one aspect, the co-stimulatory domain comprises a CD28 cytoplasmic domain having SEQ ID NO:11.

In one aspect, the intracellular signaling domain comprises a cluster of differentiation 3 zeta (CD3ζ) cytoplasmic domain having SEQ ID NO:12.

In yet another aspect, the CAR comprises an amino acid sequence having at least 90%, at least 95%, or up to 100% sequence identity to an amino acid sequence selected from the group consisting of SEQ ID NO: 14, SEQ ID NO:15, SEQ ID NO: 16, SEQ ID NO:17, SEQ ID NO: 18, SEQ ID NO: 19, SEQ ID NO:20, SEQ ID NO:21, SEQ ID NO:22, and SEQ ID NO: 58.

Also disclosed herein is a nucleic acid construct encoding a chimeric antigen receptor (CAR) disclosed herein wherein the antigen binding domain of the CAR binds to an antigen selected from the group consisting of cluster of differentiation 19 (CD19), B-cell maturation antigen (BCMA), B7 homolog 4 (B7H4), SARS-CoV-2 spike, and CD30α. In one aspect, the nucleic acid construct comprises a nucleic acid sequence selected from the group consisting of SEQ ID NO:24, SEQ ID NO:25, SEQ ID NO:26, SEQ ID NO:27, SEQ ID NO: 28, SEQ ID NO:29, SEQ ID NO:30, SEQ ID NO:31 SEQ ID NO:32 and SEQ ID NO: 59.

Also disclosed herein is an expression vector encoding a chimeric antigen receptor (CAR) disclosed herein wherein the antigen binding domain of the CAR binds to an antigen selected from the group consisting of B-cell maturation antigen (BCMA), B7 homolog 4 (B7H4), SARS-CoV-2 spike, and CD30α. In one aspect, the expression vector has a nucleic acid sequence selected from the group consisting of SEQ ID NO:33, SEQ ID NO: 34, SEQ ID NO: 35, SEQ ID NO:36, SEQ ID NO:37, SEQ ID NO:38, SEQ ID NO: 39, SEQ ID NO: 40, SEQ ID NO:41 and SEQ ID NO:42.

Further disclosed herein is a primary Natural Kill (NK) cell modified with an RNA molecule comprising one or more nucleic acids of a T7 promoter, a spacer sequence, a signal peptide sequence portion, an antigen binding domain sequence portion, a hinge region sequence portion, a transmembrane (TM) domain sequence portion and an intracellular domain sequence portion; wherein the signal peptide sequence comprises a sequence encoding cluster of differentiation 64 (CD64) and/or an IgG heavy chain variable gene (IgGHv); wherein the antigen binding domain comprises a sequence encoding an antigen binding portion that binds to an antigen selected from the group consisting of cluster of differentiation 19 (CD19), B-cell maturation antigen (BCMA), B7 homolog 4 (B7H4), SARS-CoV-2 spike, and cluster of differentiation 30 alpha (CD30α); and wherein the nucleic acid sequences are operably linked to each other as a single polynucleotide.

In one aspect of the modified primary NK cells, the intracellular domain sequence portion comprises a CD28 cytoplasmic domain having SEQ ID NO:11 and/or a cluster of differentiation 3 zeta (CD3ζ) cytoplasmic domain having SEQ ID NO:12. In one aspect the modified primary NK cell further comprises a 3′-UTR. In still another aspect, the modified primary NK cell further comprises a poly-A sequence portion.

Also disclosed herein is a method of generating modified primary CAR-NK cells comprising transfecting a primary NK cell with a recombinant nucleic acid construct disclosed herein.

Also disclosed herein is a method of immunotherapy for treating cancer in a subject in need thereof, the method comprising administering to the subject a therapeutically effective amount of a pharmaceutical composition comprising a genetically modified NK cell as disclosed herein. In one aspect, the cancer is selected from the group consisting of leukemia, acute lymphocytic leukemia, acute myelocytic leukemia, chronic leukemias, chronic myelocytic (granulocytic) leukemia, chronic lymphocytic leukemia, polycythemia vera, lymphomas, Hodgkin's disease, non-Hodgkin's disease, multiple myeloma, Waldenstrom's macroglobulinemia, heavy chain disease, solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

Also disclosed herein is a modified NK cell as disclosed herein, for use in the treatment of cancer. In one aspect, the cancer is selected from the group consisting of leukemia, acute lymphocytic leukemia, acute myelocytic leukemia, chronic leukemias, chronic myelocytic (granulocytic) leukemia, chronic lymphocytic leukemia, polycythemia vera, lymphomas, Hodgkin's disease, non-Hodgkin's disease, multiple myeloma, Waldenstrom's macroglobulinemia, heavy chain disease, solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, euroblastoma and retinoblastoma.

Also disclosed herein is a modified NK cell disclosed herein for use as a medicament.

Further disclosed herein is a pharmaceutical composition comprising a genetically-modified NK cell as disclosed herein and a pharmaceutically acceptable carrier.

### DESCRIPTION OF THE DRAWINGS

FIG. 1 is a schematic of a construct to generate an ACE2 CAR molecule (pNKW97). The extracellular domain of the ACE2 protein was used to generate a CAR molecule with a CD64 signal peptide and a 150-bp long poly A tail. Spacer sequence having SEQ ID NO:3 was used in this construct.

FIGS. 2A, 2B, 2C and 2D show expression of NKW97-150A (XL53-ACE2-Extracellular domain) in cytokine-enriched natural killer (CENK) cells. CENK cells were transfected with pNKW97 RNA using electroporation. After overnight recovery, ACE2 expression was detected using flow cytometry and a conjugated ACE2 antibody. An isotype control was used to confirm specificity of the ACE2 antibody. As shown, both expression and cell viability is very good for this construct.

FIG. 3 shows the plasmid map of pNKW97.

FIG. 4 are schematics of two constructs to generate CAR molecules targeted at the B7H4 antigen (pNKW92-93). Two versions of a mono-peptide B7H4 CAR were designed that varied in their signal peptide (CD64 or IgGHv). Spacer sequence having SEQ ID NO:3 was used in the pNKW92 construct; spacer sequence having SEQ ID NO:60 was used in the pNKW93 construct.

FIGS. 5A, 5B, 5C, 5D, 5E and 5F shows the expression of CD64 or IgGHv B7H4 VH-VL 105A(NKW92, NKW93) CAR molecules in CENK cells. CENK cells were electroporated with pNKW92 or pNKW93 mRNA. After 24 hours, B7H4 CAR expression was detected using flow and the biotinylated B7H4 followed by streptavidin-APC. Both constructs show good expression of the B7H4 CAR.

FIG. 6 shows the plasmid map of pNKW92.

FIG. 7 shows the plasmid map of pNKW93.

FIGS. 8A and 8B are schematics of four constructs to generate CAR molecules targeted at the BCMA antigen (pNKW88-91). Four versions of a mono-peptide BCMA CAR were designed that varied in their signal peptide (CD64 or IgGHv) and the order of variable heavy and light chains. Spacer sequence having SEQ ID NO:3 was used in the pNKW89 and pNKW91 constructs; spacer sequence having SEQ ID NO:60 was used in the pNKW88 and pNKW90 constructs.

FIGS. 9A, 9B, 9C, 9D and 9E show the expression of the BCMA CAR mRNA in CENK cells. Four versions of a mono-cistronic BCMA CAR were designed that varied in their signal peptide (CD64 or IgGHv) and the order of the variable heavy and light chains. CENK cells were electroporated with the mRNA from each construct. CAR BCMA expression was detected 24 hours post electroporation using a biotinylated BCMA followed by an APC-conjugated streptavidin molecule. As shown, all 4 constructs show high levels of the BCMA CAR.

FIG. 10 shows cytotoxicity of the CD64-BCMA VL-VH 150A (NKW89) CAR-transfected CENK cells on SUP-B15 BCMA target and SUP-B15 parental cells. CENK cells were electroporated with mRNA from pNKW89. After 24 hours, a Calcein AM assay was used to test the cytotoxicity of CENK-transfected cells against SUP-B15 BCMA target and control SUP-B15 parental cells. As shown, NKW89-transfected cells show specific cytotoxic activity against SUP-B15 BCMA with no activity on SUP-B15 parental cells. The control, un-transfected CENK cells show no cytotoxic activity on either SUP-B15 BCMA or parental cells.

FIG. 11 shows the plasmid map of pNKW88.

FIG. 12 shows the plasmid map of pNKW89.

FIG. 13 shows the plasmid map of pNKW90.

FIG. 14 shows the plasmid map of pNKW91.

FIGS. 15A, 15B and 15C show the results of electroporation of PB-NK cells with a Tri-peptide CD19 CAR mRNA. A stable cell line stably expressing the CD19 CAR was used as a positive control for CD19 CAR detection. Both GFP and PDL1 CAR mRNA were used as positive controls for electroporation. As shown, the Tri-peptide CAR cannot be expressed post electroporation as an RNA molecule in PB-NK cells.

FIGS. 16A, 16B, 16C and 16D show the results of stem memory T cells (Tscm cells) electroporated with a Tri-peptide CD19 CAR mRNA. Tscm cells were electroporated with the Tri-peptide CD19 CAR mRNA using 3 different electroporation protocols (E1-E3 with increasing electric pulse). As shown, the Tri-peptide CAR cannot be expressed post electroporation as an RNA molecule into memory T cells.

FIG. 17 are schematics of two constructs to generate CAR molecules targeted at the CD19 antigen (pNKW87-59). To improve expression of the CD19 CAR mRNA molecules into primary NK cells (also referred to herein as PB-NK cells) and stem memory T cells (Tscm cells), a mono-peptide CD19 construct with either CD64 or IgGHv signal peptides was designed. A 150-nucleotide long poly A tail was added to the end of each molecule to improve mRNA stability. Spacer sequence having SEQ ID NO:3 was used in these constructs.

FIGS. 18A, 18B, 18C, 18D, 18E, and 18F show expression of CD19 CAR molecule in PB-NK cells. To improve expression of the CD19 CAR mRNA molecules in primary NK cells, a mono-peptide CD19 construct with either CD64 (pNKW87) or IgGHv (pNKW59) signal peptides was designed. The template DNA was used to generate an in vitro transcribed mRNA molecule that was used for electroporation of PB-NK cells. As shown, both constructs show over 94% CD19 CAR expression 24 hours post electroporation.

FIGS. 19A-19B show cytotoxic activity of CD19 CAR transfected PB-NK cells on a CD-19-positive, SUP-B15 parental target cell line post mRNA electroporation. Cytotoxic activity of CD19 CAR transfected PB-NK cells was determined 24 hours post electroporation using a CD19 positive (SUP-B15 parental) and a CD19 negative (SUP-B15 variant) cell lines. As shown both constructs with either CD64 (pNKW87) or IgGHv (pNKW59) signal peptides show specific cytotoxicity on the CD19-positive cell line.

FIGS. 20A-20B show cytotoxic activity of CD19 CAR molecules in memory (PB-NK CIML) and control (PB-NK) cells post mRNA transfection. Cytotoxic activity of CD19 CAR transfected memory (PB-NK CIML) or control (PB-NK) cells was determined 24 hours post electroporation using a CD19 positive (SUP-B15 parental) and a CD19 negative (SUP-B15 variant) cell lines. As shown, CD19-CAR-transfected PB-NK CIML cells show comparable cytotoxic activity to CD19-CAR-transfected, control PB-NK cells. This will further enhance directing CIML cells towards specific targeting of the CD19 positive tumor cells.

FIGS. 21A, 21B, 21C, 21D, and 21E show the results of monitoring CD19 CAR expression 24-72 hours post electroporation of activated T cells with CD19 CAR constructs. To improve expression of the CD19 CAR mRNA molecules in primary lymphocytes, a mono-peptide CD19 construct with either CD64 (pNKW87—FIGS. 21B and 21C) or IgGH (pNKW59—FIGS. 21D and 21E) signal peptides was designed. The template DNA served as a template for an in vitro transcribed mRNA molecule that was further used for electroporation of primary cells.

FIGS. 22A, 22B, 22C, 22D and 22E show the results of monitoring CD19 expression 24-72 hours post electroporation of Tscm cells with CD19 CAR constructs. To improve expression of the CD19 CAR mRNA molecules in memory T cells, a mono-cistronic CD19 construct with either CD64 (pNKW87-FIGS. 22B and 22C) or IgGHv (pNKW59—FIGS. 22D and 22E) signal peptides was designed. The template DNA served as a template for an in vitro transcribed mRNA molecule that was further used for electroporation of Tscm cells. As shown, both constructs show high levels of CD19 CAR expression even after 72 hours post electroporation.

FIG. 23 shows the plasmid map of pNKW59.

FIG. 24 shows the plasmid map of pNKW87.

FIG. 25 is a schematic of a construct to generate CAR molecules targeted at the CD30 alpha (CD30α) antigen (pNKW95). A mono-cistronic CD30α CAR were designed that contained a CD64 signal peptide and a 150-bp long poly A tail. Spacer sequence having SEQ ID NO:3 was used in the construct.

FIGS. 26A, 26B, 26C, 26D, 26E and 26F show the expression of alpha-CD30 CD64-VL-VH CAR (NKW95-150A) in CENK cells. CENK cells were transfected with pXL46 (short poly A) or pNKW95 RNA (150A) using electroporation. After overnight recovery, CD30α CAR expression was detected using flow cytometry, and a biotinylated CD30 followed by streptavidin-APC. The pNKW95 construct shows more expression compared to the parental pXL46.

FIG. 27 shows the plasmid map of pNKW95.

### DETAILED DESCRIPTION

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry are those well-known and commonly used in the art.

All publications, patents, and published patent applications referred to in this application are specifically incorporated by reference herein. In case of conflict, the present specification, including its specific definitions, will control.

As disclosed herein, the inventors have determined a novel combination of specific domains and stabilizing elements for high expression of a chimeric antigen mRNA molecule in primary NK cells. Specially, as disclosed herein, the novel combination comprises an extracellular domain comprising signal peptide, an antigen binding domain, a hinge, a transmembrane (TM), and comprising an intracellular domain comprising at least one co-stimulatory domain and an intracellular signaling domain. In addition, the CAR has modifications in the 3′ untranslated region (UTR) for optimal expression of the in-vitro transcribed RNA in NK cells. Initial results by the inventors have shown that many CAR molecules that have been easily expressed in NK cell lines, show minimal or no expression after in vitro transcription and electroporation into primary NK cells. Most CAR technology uses viral vectors for the delivery of a DNA molecule into the cells. The viral DNA enters the nucleus and can integrate into the host genome with a preference for the transcriptionally active sites. As disclosed herein, the inventors have utilized an alternative approach that uses an mRNA molecule that does not integrate into the genome thereby avoiding the risks associated with the oncogenic potentials of viral gene deliveries. Besides safety, another advantage of using a CAR mRNA molecule as disclosed herein (as compared to DNA) is the quicker response that results as mRNA is quickly translated upon entry into the cytoplasm. One disadvantage of mRNA is that it degrades easily, however, the inventors have addressed this disadvantage by prolonging the half-life of the mRNA constructs disclosed herein by introducing stabilizing elements in the 5′ end of each CAR molecule. Further as demonstrated in the figures and examples provided herein, the inventors have also shown successful expression of the CAR mRNA in memory NK and T cells.

The CAR molecules described herein target cancer surface markers, including but not limited to CD19, BCMA and CD30, as well as checkpoint inhibitors or their ligands including but not limited to B7H4. The DNA template vectors serve as templates for in vitro synthesis of an mRNA molecule that is delivered to the primary NK cells disclosed herein for the purpose of immunotherapy in cancer patients. In vitro transcription can be initiated at a promoter, such as the T7 promoter, using the bacteriophage T7 RNA polymerase. The T7 promoter is upstream of a spacer sequence (SEQ ID NO:3 or SEQ ID NO:60) comprising a Kozak sequence (SEQ ID NO: 45) that is required for the initiation of translation. A short signal peptide (15-amino acids) from the CD64 or IgGHv protein marks the N-terminus of the CAR protein. The signal peptide is recognized by a signal recognition peptide (SRP) in the cytosol that delivers the nascent polypeptide chain from the cytosol to the endoplasmic reticulum. The CAR binding site is a heterodimer of variable light and heavy chain domains. The two domains are connected to each other via a 20 amino acid (aa) linker. The hinge and the TM domains of the molecule are derived from the CD28 protein. The hinge region provides a range of motion and flexibility for the binding domain, while the TM region/domain allows correct membrane insertion. The intracellular domain comprises at least one co-stimulatory domain and an intracellular signaling domain. The co-stimulatory domain comprises the cytoplasmic domain of CD28, while the intracellular signaling domain comprises the cytoplasmic domain of CD3ζ. The co-stimulatory and intracellular signaling domains are engaged in intracellular signaling pathways that enhance cytotoxic activities of the transfected cells. The 3′ UTR of the constructs disclosed herein is a 94-bp sequence from 3′ UTR of Mus Musculus hemoglobin alpha gene followed a 150-bp poly A stretch confers stability to the RNA molecule. Alternatives for the 3′ UTR regions can be used in the CAR constructs. These include but are not limited to 3′ UTRs from human beta globin or 3′ UTRs from genes that are highly expressed in NK cells. Examples of genes that are highly expressed in NK cells include but are not limited to natural cytotoxicity receptors (NCR) such as NKp46, NKp30, NKp44; or c-lectin like activating immunoreceptors such as NKG2D and 2B4. The 3′ UTRs (as well as their alternatives) can be introduced into the CAR constructs and can improve stability of the mRNA CAR molecules.

The constructs that are disclosed herein are novel in that they have a high binding affinity for specific cancer surface markers, checkpoint inhibitors and/or their ligands. Further, the constructs are comprised of cytoplasmic domains of CD28 and CD3ζ that result in enhanced cytotoxic activity against target cells. Also, the constructs are mRNA based and thus there is no concern regarding integration of the constructs into the host genome.

The signal peptide comprises a CD64 and/or an IgGHv signal peptide. In one aspect, the signal peptide comprises SEQ ID NO: 1 or SEQ ID NO:2.

The antigen binding domain binds to an antigen selected from the group consisting of CD19, BCMA, B7H4, SARS-CoV-2 spike and CD30α.

In one aspect, the antigen binding domain that binds to CD19 comprises at least 90%, at least 95%, or up to 100% sequence identity to SEQ ID NO:4.

In still another aspect, the antigen binding domain that binds to BCMA comprises at least 90%, at least 95%, or up to 100% sequence identity to SEQ ID NO:5 or SEQ ID NO: 6.

In yet another aspect, the antigen binding domain that binds to B7H4 comprises at least 90%, at least 95%, or up to 100% sequence identity to SEQ ID NO:7.

In one aspect, the antigen binding domain that binds to SARS-CoV-2 spike comprises at least 90%, at least 95%, or up to 100% sequence identity to SEQ ID NO:8.

In one aspect, the antigen binding domain that binds to CD30α comprises at least 90%, at least 95%, or up to 100% sequence identity to SEQ ID NO:57.

In one aspect, the hinge region is a CD28 hinge region having SEQ ID NO:9.

In one aspect, the TM domain is a CD28 TM domain having SEQ ID NO: 10.

In one aspect, the co-stimulatory domain comprises a CD28 cytoplasmic domain having SEQ ID NO:11. In yet another aspect, the co-stimulator domain comprises 2B4, 4-1BB (also referred to as CD137 or TNFRS9) and/or OX40. Still further, additional co-stimulatory domains can be added to the constructs. Also contemplated is swapping/changing the order/location of the co-stimulatory domains within the construct.

In one aspect, the intracellular signaling domain comprises a CD3ζ cytoplasmic domain having SEQ ID NO:12.

As disclosed herein, the CAR comprises an amino acid sequence having at least 80%, at least 90%, at least 95%, or up to 100% sequence identity to an amino acid sequence selected from the group consisting of SEQ ID NO: 14, SEQ ID NO: 15, SEQ ID NO: 16, SEQ ID NO: 17, SEQ ID NO: 18, SEQ ID NO: 19, SEQ ID NO:20, SEQ ID NO:21, SEQ ID NO:22 and SEQ ID NO:58.

Another embodiment is a nucleic acid construct encoding a CAR disclosed herein, wherein the CAR comprises an extracellular domain comprising a signal peptide, an antigen binding domain, a hinge region, and a TM domain; and comprising at least one co-stimulatory domain and intracellular signaling domain; wherein the signal peptide comprises a CD64 and/or an IgGHv signal peptide; and wherein the antigen binding domain binds to an antigen selected from the group consisting of CD19, BCMA, B7H4, SARS-CoV-2 spike and CD30α, as well as variants thereof. In one aspect, the nucleic acid construct comprises a nucleic acid sequence selected from the group consisting of SEQ ID NO:24, SEQ ID NO:25, SEQ ID NO:26, SEQ ID NO:27, SEQ ID NO:28, SEQ ID NO:29, SEQ ID NO: 30, SEQ ID NO:31 SEQ ID NO:32 and SEQ ID NO:59. In another aspect, a SARS-CoV-2-antigen refers to a SARS-CoV-2 protein, and a variant thereof. Examples of suitable SARS-CoV-2 proteins that may be used as, or to produce, SARS-CoV-2 antigens include, but are not limited to, main protease (M^{PRO}, also known as Chain A 3C-like proteinase or 3C-like proteinase), SARS-CoV-2 nucleocapsid protein (N protein), SARS-CoV-2 membrane protein (M protein), SARS-CoV-2 envelope protein (E protein), and SARS-CoV-2 spike protein (S protein).

Another embodiment disclosed herein, is an expression vector encoding a CAR as disclosed herein, wherein the CAR comprises an extracellular domain comprising a signal peptide, an antigen binding domain, a hinge region, and a TM domain; and an intracellular domain comprising at least one co-stimulatory domain and an intracellular signaling domain; wherein the signal peptide comprises a CD64 and/or an IgGHv signal peptide; and wherein the antigen binding domain binds to an antigen selected from the group consisting of CD19, BCMA, B7H4, SARS-CoV-2 spike and CD30α. In one aspect, the expression vector has a nucleic acid sequence selected from the group consisting of SEQ ID NO:33, SEQ ID NO: 34, SEQ ID NO:35, SEQ ID NO:36, SEQ ID NO:37, SEQ ID NO:38, SEQ ID NO: 39, SEQ ID NO:40, SEQ ID NO:41, SEQ ID NO:42.

A further embodiment disclosed herein, is a modified primary NK cell expressing a CAR as disclosed herein, wherein the CAR comprises an extracellular domain comprising a signal peptide, an antigen binding domain, a hinge region, and a TM domain; and an intracellular domain comprising at least one co-stimulatory domain and intracellular signaling domain; wherein the signal peptide comprises a CD64 and/or an IgGHv signal peptide; and wherein the antigen binding domain binds to an antigen selected from the group consisting of CD19, BCMA, B7H4, SARS-CoV-2 spike, and CD30α.

Another embodiment disclosed herein is a method for making a genetically modified NK cell comprising the step of introducing a CAR mRNA molecule transcribed off of an expression vector as disclosed herein.

It is also possible to add other cytokine genes to the same construct (as a bi- or tri-peptide) which can improve activity of the RNA CAR molecule. Such cytokine genes include IL-15.

Also contemplated is the introduction of domains from activating NK receptors, such as NKG2D, into the CAR molecules disclosed herein for improving their activity. NKG2D is a transmembrane protein belonging to the NKG2 family of C-type lectin-like receptors.

A further embodiment disclosed herein, is a method of immunotherapy for treating cancer in a subject in need thereof. The method comprising administering to the subject a pharmaceutical composition comprising a genetically modified NK cell as disclosed herein and pharmaceutically acceptable carrier. Another embodiment is the use of a genetically modified NK cell as disclosed herein and pharmaceutically acceptable carrier for treating cancer.

Another embodiment is a pharmaceutical composition comprising a modified NK cell as disclosed herein and a pharmaceutically acceptable carrier.

Throughout this specification, “comprise” or variations such as “comprises” or “comprising” imply the inclusion of a stated integer (or components) or group of integers (or components), but not the exclusion of any other integer (or component) or group of integers (or components).

The singular forms “a,” “an,” and “the” include the plurals unless the context clearly dictates otherwise.

“Including” means “including but not limited to.” “Including” and “including but not limited to” are used interchangeably.

“Pharmaceutically acceptable carrier” refers to a non-toxic carrier that may be administered to a patient-together with compositions described herein-and which does not destroy the pharmacological activity of the active agents within the composition. “Excipient” refers to an additive in a formulation or composition that is not a pharmaceutically active ingredient.

“Pharmaceutically effective amount” refers to an amount effective to treat a patient, e.g., effecting a beneficial and/or desirable alteration in the general health of a patient suffering from a disease (including but not limited cancer). Treating includes, but is not limited to, killing cells, preventing the growth of new cells, improving vital functions of a patient, improving the well-being of the patient, decreasing pain, improving appetite, improving patient weight, and any combination thereof. A “pharmaceutically effective amount” also refers to the amount required to improve a patient's clinical symptoms.

“Peptide” and “polypeptide” are used synonymously herein to refer to polymers constructed from amino acid residues. “Amino acid residue” as used herein refers to any naturally occurring amino acid (L or D form), non-naturally occurring amino acid, or amino acid mimetic (such as peptide monomer).

“Identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window. The degree of amino acid or nucleic acid sequence identity for purposes of the present disclosure is determined using the BLAST algorithm, described in Altschul et al. (1990) J. Mol. Biol. 215:403-10. This algorithm identifies high scoring sequence pairs (HSPS) by identifying short words of length W in the query sequence, which either match or satisfy some positive-valued threshold score T when aligned with a word of the same length in a database sequence. T is referred to as the neighborhood word score threshold (Altschul et al., (1990) J. Mol. Biol. 215:403-10). Initial neighborhood word hits act as seeds for initiating searches to find longer HSPs containing them. The word hits are then extended in both directions along each sequence for as far as the cumulative alignment score can be increased. Cumulative scores are calculated for nucleotides sequences using the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below due to the accumulation of one or more negative-scoring residue alignments; or the end of either sequence is reached. For determining the percent identity of an amino acid sequence the BLASTP settings are: word length (W), 3; expectation (E), 10; and the BLOSUM62 scoring matrix. For analysis of nucleic acid sequences, the BLASTN program settings are word length (W), 11; expectation (E), 10; M=5; N =-4; and a comparison of both strands. The TBLASTN program (using a protein sequence to query nucleotide sequence databases) uses a word length (W) of 3, an expectation (E) of 10, and a BLOSUM 62 scoring matrix. (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Nat'l. Acad. Sci. USA 90:5873-87). The smallest sum probability (P (N)), provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01.

The “length” of a polypeptide is the number of amino acid residues linked end-to-end that constitute the polypeptide, excluding any non-peptide linkers and/or modifications that the polypeptide may contain.

Hydrophobic amino acid residues are characterized by a functional group (“side chain”) that has predominantly non-polar chemical properties. Such hydrophobic amino acid residues can be naturally occurring (L or D form) or non-naturally occurring. Alternatively, hydrophobic amino acid residues can be amino acid mimetics characterized by a side chain that has predominantly non-polar chemical properties. Conversely, hydrophilic amino acid residues are characterized by a side chain that has predominantly polar (charged or uncharged) chemical properties. Such hydrophilic amino acid residues can be naturally occurring (L or D form) or non-naturally occurring. Alternatively, hydrophilic amino acid residues can be amino acid mimetics characterized by a side chain that has predominantly polar (charged or uncharged) chemical properties. Suitable non-naturally occurring amino acid residues and amino acid mimetics are known in the art. See, e.g., Liang et al. (2013) PLOS ONE 8 (7): e67844.

Although most amino acid residues can be considered as either hydrophobic or hydrophilic, a few-depending on their context-can behave as either hydrophobic or hydrophilic. For example, the relatively weak non-polar characteristics of glycine, proline, and cysteine enable them each sometimes to function as hydrophilic amino acid residues. Conversely, the bulky, slightly hydrophobic side chains of histidine and arginine enable them each sometimes to function as hydrophobic amino acid residues.

Unless otherwise specified, each embodiment disclosed herein may be used alone or in combination with any one or more other embodiments herein.

“Transfection” refers to introduction of foreign nucleic acid into eukaryotic cells. Transfection may be accomplished by a variety of means known to the art, including electroporation, polymers (nanoparticles), calcium phosphate-DNA co-precipitation, DEAE-dextran-mediated transfection, polybrene-mediated transfection, microinjection, liposome fusion, lipofection, protoplast fusion, and biolistics.

“Stable transfection” or “stably transfected” refers to the introduction and integration of foreign nucleic acid, DNA, into the genome of the transfected cell.

The term variant refers to a protein, or fragment thereof, having an amino acids sequence that is similar, but not identical, to a referenced sequence (e.g., a SARS-CoV-2 protein sequence), wherein the activity of the variant protein is not significantly altered. These variations in sequence can be naturally occurring variations or they can be engineered through the use of technique known to those skilled in the art. Examples of suitable variations include, but are not limited to, amino acid deletions, insertions, substitutions, and combinations thereof.

Amino acids can be classified into groups based on their physical properties. Examples of such groups include, but are not limited to, charged amino acids, uncharged amino acids, polar uncharged amino acids, and hydrophobic amino acids. Preferred variants are those in which an amino acid is substituted with an amino acid from the same group. Such substitutions are referred to as conservative substitutions.

Naturally occurring residues may be divided into classes based on common side chain properties:
1) hydrophobic: Met, Ala, Val, Leu, Ile;
2) neutral hydrophilic: Cys, Ser, Thr;
3) acidic: Asp, Glu;
4) basic: Asn, Gln, His, Lys, Arg;
5) residues that influence chain orientation: Gly, Pro; and
6) aromatic: Trp, Tyr, Phe.

Non-conservative substitutions may involve the exchange of a member of one of these classes for a member from another class.

Methods and uses are also provided for treating or ameliorating the symptoms of cancer and/or to treating a cancer or a tumor in an individual. The method and/or use comprises administering to the subject a therapeutically effective amount of the modified NK cells as disclosed herein or a composition comprising modified NK cells as disclosed herein to a patient in need thereof. The administration is contemplated to treat the cancer, reduces the size of the tumor in the subject, or reduce cancer metastasis in the subject. One embodiment is a modified NK cell as disclosed herein for use in the treatment of cancer. Yet still another embodiment is a modified NK cell as disclosed herein for use as a medicament.

The term “cancer” refers to all types of cancer, neoplasm, or malignant tumors found in mammals, including leukemia, carcinomas and sarcomas. Exemplary cancers include cancer of the brain, breast, cervix, colon, head & neck, liver, kidney, lung, non-small cell lung, melanoma, mesothelioma, ovary, sarcoma, stomach, uterus and Medulloblastoma. Additional examples include, Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas, and prostate cancer.

The terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms subject, patient, individual, etc. are not intended to be limiting and can be generally interchanged. That is, an individual described as a patient does not necessarily have a given disease but may be merely seeking medical advice. As used throughout, a subject can be a vertebrate, more specifically a mammal (e.g., a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, patient, individual and subject may be used interchangeably and these terms are not intended to be limiting. That is, an individual described as a patient does not necessarily have a given disease, but may be merely seeking medical advice. The terms patient or subject include human and veterinary subjects.

Reference herein to “therapeutic” and “prophylactic” is to be considered in their broadest contexts. “Therapeutic” does not necessarily imply that a mammal is treated until total recovery. Similarly, “prophylactic” does not necessarily mean that the subject will not eventually contract a disease condition. The term “prophylaxis” may be considered as reducing the severity of onset of a particular condition. Therapy may also reduce the severity of an existing condition or the frequency of acute attacks. As used herein, “treat,” “treating,” and similar words mean stabilizing and/or reducing the symptoms of a disease or condition. In some aspects, the compositions disclosed herein can prevent the occurrence of a disease or condition, or cure a medical condition or disease, which is separate from treating.

Routes and frequency of administration of the therapeutic compositions described herein, as well as dosage, will vary from individual to individual, and from disease to disease, and may be readily established using standard techniques. In general, the pharmaceutical compositions may be administered by injection (e.g., intracutaneous, intramuscular, intravenous or subcutaneous), intranasally (e.g., by aspiration), in pill form (e.g. swallowing, suppository for vaginal or rectal delivery).

As described herein, the compositions of the invention are suitable for parenteral administration. These compositions may be administered, for example, intraperitoneally, intravenously, or intrathecally, intracranialy, intradermally, intramuscularly, intraocularly, intrathecaly, intracerebrally, intranasally, transmucosally, by infusion, orally, rectally, via iv drip, patch and implant, parenterally, orthotopically, subcutaneously, topically, nasally, orally, sublingually, intraocularly, by means of an implantable depot, using nanoparticle-based delivery systems, microneedle patch, microspheres, beads, osmotic or mechanical pumps, and/or other mechanical means. Optionally, the NK cells are administered parenterally. Optionally, the NK cells are administered intravenously. Optionally, the NK cells are administered peritumorally. One of skill in the art would appreciate that a method of administering the composition of the invention would depend on factors such as the age, weight, and physical condition of the patient being treated, and the disease or condition being treated. The skilled worker would, thus, be able to select a method of administration optimal for a patient on a case-by-case basis.

The modified NK cells as disclosed herein can be administered to a subject by absolute numbers of cells, e.g., said subject can be administered from about 1000 cells/injection to up to about 10 billion cells/injection, such as at about, at least about, or at most about, 1×10¹⁰, 1×10⁹, 1×10⁸, 1×10⁷, 5×10⁷, 1×10⁶, 5×10⁶, 1×10⁵, 5×10⁵, 1×10⁴, 5×10⁴, 1×10³, 5×10³ (and so forth) NK cells per injection, or any ranges between any two of the numbers, end points inclusive. Optionally, from 1×10⁸ to 1×10¹⁰ cells are administered to the subject. Optionally, the cells are administered one or more times weekly for one or more weeks. Optionally, the cells are administered once or twice weekly for 1, 2, 3, 4, 5, 6, 7, 8, 9, 10 or more weeks.

In another embodiment, the total dose may also calculated by m² of body surface area. The subject may be administered from about 1000 cells/injection/m² to up to about 10 billion cells/injection/m², such as at about, at least about, or at most about, 1×10¹⁰/m², 1×10⁹/m², 1×10⁸/m², 1×10⁷/m², 5×10⁷/m², 1×10⁶/m², 5×10⁶/m², 1×10⁵/m², 5×10⁵/m², 1×10⁴/m², 5×10⁴/m², 1×10³/m², 5×10³/m²(and so forth) NK cells per injection, or any ranges between any two of the numbers, end points inclusive. Optionally, from 1×10³ to 1×10¹⁰, per m² of the NK cells are administered to the subject. Optionally, 2×10⁹ per m², of the NK cells are administered to the subject.

In some embodiments, NK cells are administered in a composition comprising NK cells and a medium, such as human serum or an equivalent thereof. The medium may comprise human serum albumin and/or human plasma. Optionally, the medium comprises about 1% to about 15% human serum or human serum equivalent. Optionally, the medium comprises about 1% to about 10% human serum or human serum equivalent. Optionally, the medium comprises about 1% to about 5% human serum or human serum equivalent. Optionally, the medium comprises about 2.5% human serum or human serum equivalent. Optionally, the serum is human AB serum. Optionally, a serum substitute that is acceptable for use in human therapeutics is used instead of human serum. Such serum substitutes may be known in the art. Optionally, NK cells are administered in a composition comprising NK cells and an isotonic liquid solution that supports cell viability. Optionally, NK cells are administered in a composition that has been reconstituted from a cryopreserved sample.

According to the methods and uses provided herein, the subject is administered an effective amount or therapeutically effective amount of one or more of the agents provided herein. The terms effective amount, therapeutically effective amount and effective dosage are used interchangeably. The term effective amount is defined as any amount necessary to produce a desired physiologic response (e.g., reduction of inflammation). Effective amounts and schedules for administering the agent may be determined empirically by one skilled in the art. The dosage ranges for administration are hose large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 22nd Edition, Gennaro, Editor (2012), and Pickar, Dosage Calculations (1999)).

Compositions suitable for injectable use include sterile aqueous solutions (where water soluble) and sterile powders for the extemporaneous preparation of sterile injectable solutions. The carrier can be a solvent or dispersion medium containing, for example, water, ethanol, polyol (for example, glycerol, propylene glycol and liquid polyethylene glycol, and the like), suitable mixtures thereof and vegetable oils. The prevention of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by, for example, filter sterilization or sterilization by other appropriate means. Dispersions may be prepared by incorporating the various sterilized active ingredients into a sterile vehicle which contains the basic dispersion medium and the required other ingredients from those enumerated above. In the case of sterile powders for the preparation of sterile injectable solutions, a preferred method of preparation includes vacuum drying and the freeze-drying technique which yield a powder of the active ingredient plus any additional desired ingredient from a previously sterile-filtered solution.

When the active ingredients are suitably protected, they may be orally administered, for example, with an inert diluent or with an assimilable edible carrier, or it may be enclosed in hard or soft shell gelatin capsule, or it may be compressed into tablets. For oral therapeutic administration, the active compound may be incorporated with excipients and used in the form of ingestible tablets, buccal tablets, troches, capsules, elixirs, suspensions, syrups, wafers, and the like.

In conjunction with any of the foregoing methods and uses, the compositions can be administered in combination with another drug. In each case, the composition can be administered prior to, at the same time as, or after the administration of the other drug. In accordance with the methods described herein, more than one compound or composition may be co-administered with one or more other compounds, such as known chemotherapies, anti-viral compounds or molecules as well as antibiotics, chloroquine, hydroxychloroquine, known drugs for treating pneumonia, an analgesic (such as lidocaine or paracetoamol), an anti-inflammatory (such as betamethasone, non-steroid anti-inflammatory drugs (NSAIDs), acetaminophen, ibuprofen, naproxen), and/or other suitable drugs. The provided methods may be further combined with other tumor therapies such as radiotherapy, surgery, hormone therapy and/or immunotherapy. Thus, the provided methods can further include administering one or more additional therapeutic agents to the subject. Suitable additional therapeutic agents include, but are not limited to, analgesics, anesthetics, analeptics, corticosteroids, anticholinergic agents, anticholinesterases, anticonvulsants, antineoplastic agents, allosteric inhibitors, anabolic steroids, antirheumatic agents, psychotherapeutic agents, neural blocking agents, anti-inflammatory agents, antihelmintics, antibiotics, anticoagulants, antifungals, antihistamines, antimuscarinic agents, antimycobacterial agents, antiprotozoal agents, antiviral agents, dopaminergics, hematological agents, immunological agents, muscarinics, protease inhibitors, vitamins, growth factors, and hormones. The choice of agent and dosage can be determined readily by one of skill in the art based on the given disease being treated. Optionally, the additional therapeutic agent is octreotide acetate, interferon, pembrolizumab, glucopyranosyl lipid A, carboplatin, etoposide, or any combination thereof.

In one aspect, the compositions can be administered with N-803 (also referred to as “ALT-803”). In one aspect, the N-803 can be administered together with or separately from the pharmaceutical composition comprising the peptide(s) as disclosed herein.

In some embodiments, the additional therapeutic entity may be selected from the group consisting of a viral cancer vaccine, a bacterial cancer vaccine, a yeast cancer vaccine, an antibody, a stem cell transplant, and a tumor targeted cytokine.

Optionally, the additional therapeutic agent is a chemotherapeutic agent. A chemotherapeutic treatment regimen can include administration to a subject of one chemotherapeutic agent or a combination of chemotherapeutic agents. Chemotherapeutic agents include, but are not limited to, alkylating agents, anthracyclines, taxanes, epothilones, histone deacetylase inhibitors, inhibitors of Topoisomerase I, inhibitors of Topoisomerase II, kinase inhibitors, monoclonal antibodies, nucleotide analogs and precursor analogs, peptide antibiotics, platinum-based compounds, retinoids, and vinca alkaloids and derivatives. Optionally, the chemotherapeutic agent is carboplatin.

“Co-administered” conveys simultaneous administration in the same formulation or in two different formulations via the same or different routes or sequential administration by the same or different routes. “Sequential” administration conveys a time difference of seconds, minutes, hours, or days between the administration of the two or more separate compounds

N-803 is an interleukin-15 (IL-15) superagonist complex. In preclinical studies, IL-15 exhibits potent antitumor activities against well-established tumors. In addition, N-803 can synergistically enhance the antibody-dependent cellular cytotoxicity (ADCC) activity of therapeutic antibodies and anti-tumor activities of checkpoint inhibitors, such as anti-PD-1, anti-PD-L1, and anti-CTLA antibodies (Rhode et al., Cancer Immunol Res., 2016).

The useful concentration of N-803 is one that is suitable for that subject. One of skill in the art would appreciate that different individuals may require different total amounts of N-803. In some embodiments, the amount of N-803 is a pharmaceutically effective amount. The skilled worker would be able to determine the amount of N-803 in a composition needed to treat a subject based on factors such as, for example, the age, weight, and physical condition of the subject. A pharmaceutically effective amount of N-803 can be from about 1 pg compound/kg body weight to about 20 μg/kg compound/kg body weight; or from about 0.1 μg/kg to 20 μg/kg; or from about 1 μg/kg body weight to about 1 mg compound/kg body weight, or from about 1 mg/kg body weight to about 5000 mg/kg body weight; or from about 5 mg/kg body weight to about 4000 mg/kg body weight or from about 10 mg/kg body weight to about 3000 mg/kg body weight; or from about 50 mg/kg body weight to about 2000 mg/kg body weight; or from about 100 mg/kg body weight to about 1000 mg/kg body weight; or from about 150 mg/kg body weight to about 500 mg/g body weight. Preferably, 100 pk/kg to 20 μg/kg. In other embodiments this dose may be about 0.1,.5, 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 mg/kg body weight. In other embodiments, doses may be in the range of about 5 mg compound/kg body to about 20 mg compound/kg body. In other embodiments the doses may be about 8, 10, 12, 14, 16 or 18 mg/kg body weight.

Combinations of agents or compositions can be administered either concomitantly (e.g., as a mixture), separately but simultaneously (e.g., via separate intravenous lines) or sequentially (e.g., one agent is administered first followed by administration of the second agent). Thus, the term combination is used to refer to concomitant, simultaneous, or sequential administration of two or more agents or compositions. The course of treatment is best determined on an individual basis depending on the particular characteristics of the subject and the type of treatment selected. The treatment, such as those disclosed herein, can be administered to the subject on a daily, twice daily, bi-weekly, monthly, or any applicable basis that is therapeutically effective. The treatment can be administered alone or in combination with any other treatment disclosed herein or known in the art. The additional treatment can be administered simultaneously with the first treatment, at a different time, or on an entirely different therapeutic schedule (e.g., the first treatment can be daily, while the additional treatment is weekly).

In some embodiments, it may be beneficial to include one or more excipients in a composition. One of skill in the art would appreciate that the choice of any one excipient may influence the choice of any other excipient. For example, the choice of a particular excipient may preclude the use of one or more additional excipients because the combination of excipients would produce undesirable effects. One of skill in the art would be able to determine empirically which excipients, if any, to include in the formulations or compositions disclosed herein. Excipients may include, but are not limited to, co-solvents, solubilizing agents, buffers, pH adjusting agents, bulking agents, surfactants, encapsulating agents, tonicity-adjusting agents, stabilizing agents, protectants, and viscosity modifiers. In some embodiments, it may be beneficial to include a pharmaceutically acceptable carrier.

In some embodiments, it may be beneficial to include a solubilizing agent. Solubilizing agents may be useful for increasing the solubility of any of the components of the formulation or composition, including a peptide disclosed herein or an excipient. The solubilizing agents described herein are not intended to constitute an exhaustive list, but are provided merely as exemplary solubilizing agents that may be used. In certain embodiments, solubilizing agents include, but are not limited to, ethyl alcohol, tert-butyl alcohol, polyethylene glycol, glycerol, methylparaben, propylparaben, polyethylene glycol, polyvinyl pyrrolidone, and any pharmaceutically acceptable salts and/or combinations thereof.

The pH may be any pH that provides desirable properties for the composition. Desirable properties may include, for example, peptide stability, increased peptide retention as compared to compositions at other pHs, and improved filtration efficiency.

In some embodiments, it may be beneficial to include a tonicity-adjusting agent. The tonicity of a liquid composition is an important consideration when administering the composition to a patient, for example, by parenteral administration. Tonicity-adjusting agents, thus, may be used to help make a composition suitable for administration. Tonicity-adjusting agents are well known in the art. Accordingly, the tonicity-adjusting agents described herein are not intended to constitute an exhaustive list but are provided merely as exemplary tonicity-adjusting agents that may be used. Tonicity-adjusting agents may be ionic or non-ionic and include, but are not limited to, inorganic salts, amino acids, carbohydrates, sugars, sugar alcohols, and carbohydrates. Exemplary inorganic salts may include sodium chloride, potassium chloride, sodium sulfate, and potassium sulfate. An exemplary amino acid is glycine. Exemplary sugars may include sugar alcohols such as glycerol, propylene glycol, glucose, sucrose, lactose, and mannitol.

In some embodiments, it may be beneficial to include a stabilizing agent. Stabilizing agents help increase the stability of peptides in compositions of the invention.

In some embodiments, it may be beneficial to include a protectant. Protectants are agents that protect a pharmaceutically active ingredient (e.g., a peptide as disclosed herein) from an undesirable condition (e.g., instability caused by freezing or lyophilization, or oxidation). Protectants can include, for example, cryoprotectants, lyoprotectants, and antioxidants. Cryoprotectants are useful in preventing loss of potency of an active pharmaceutical ingredient (e.g., a peptide as disclosed herein) when a formulation is exposed to a temperature below its freezing point. For example, a cryoprotectant could be included in a reconstituted lyophilized formulation so that the formulation could be frozen before dilution for intravenous (IV) administration. Cryoprotectants are well known in the art. Accordingly, the cryoprotectants described herein are not intended to constitute an exhaustive list, but are provided merely as exemplary cryoprotectants that may be used. Cryoprotectants include, but are not limited to, solvents, surfactants, encapsulating agents, stabilizing agents, viscosity modifiers, and combinations thereof. Cryoprotectants may include, for example, disaccharides (e.g., sucrose, lactose, maltose, and trehalose), polyols (e.g., glycerol, mannitol, sorbitol, and dulcitol), glycols (e.g., ethylene glycol, polyethylene glycol, propylene glycol).

Lyoprotectants are useful in stabilizing the components of a lyophilized formulation or composition. For example, a peptide as disclosed herein could be lyophilized with a lyoprotectant prior to reconstitution. Lyoprotectants are well known in the art. Accordingly, the lyoprotectants described herein are not intended to constitute an exhaustive list, but are provided merely as exemplary lyoprotectants that may be used. Lyoprotectacts include, but are not limited to, solvents, surfactants, encapsulating agents, stabilizing agents, viscosity modifiers, and combinations thereof. Exemplary lyoprotectants may be, for example, sugars and polyols, trehalose, sucrose, dextran, and hydroxypropyl-beta-cyclodextrin are non-limiting examples of lyoprotectants.

Antioxidants are useful in preventing oxidation of the components of a composition. Oxidation may result in aggregation of a drug product or other detrimental effects to the purity of the drug product or its potency. Antioxidants are well known in the art. Accordingly, the antioxidants described herein are not intended to constitute an exhaustive list but are provided merely as exemplary antioxidants that may be used. Antioxidants may be, for example, sodium ascorbate, citrate, thiols, metabisulfite, and combinations thereof.

Variations, modifications, and other implementations of what is described herein will occur to those of ordinary skill without departing from the spirit and the scope of the present disclosure. Accordingly, the ensuing claims not to be limited only to the preceding illustrative description.

Each of the embodiments described herein may be combined individually or in combination with one or more other embodiments of the invention.

Those skilled in the art will recognize or be able to ascertain using no more than routine experimentation, numerous equivalents to the compounds, compositions, and methods of use thereof described herein. Such equivalents are considered to be within the scope of the compositions and methods disclosed herein.

The contents of all references, patents and published patent applications cited throughout this Application, as well as their associated figures are hereby incorporated by reference in their entirety.

### EXAMPLES

The following examples are put forth to provide those of ordinary skill in the art with a complete disclosure and description of how to make and use the embodiments and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, and temperature is in degrees Celsius. Standard abbreviations are used.

### Example 1

This example describes the generation of the RNA constructs disclosed herein. The vectors encoding the CAR constructs disclosed herein were generated by Gibson assembly of either PCR fragments or gene blocks. The 150-polyA tail was inserted at the 3′ end of each CAR molecule using engineered restriction sites. DNA vectors were fully sequenced, and the length of the poly A tail was determined. The CAR DNA was next linearized with SapI and the digested DNA was further purified. RNA was transcribed from the purified DNA template using T7 polymerase. RNA was next precipitated using lithium chloride and was subjected to electrophoresis to determine size and purity.

TABLE 1
pNKW97

| | | pNKW97(ACE2 |
|---|---|---|
| | Nucleotide | EC domain CAR) |
| | SEQ ID NO: | SEQ ID NO: 33 |
| Size | | 8764 bp |
| EF1a promoter | SEQ ID NO: 43 | 9-1190 |
| T7 promoter | SEQ ID NO: 44 | 1198-1216 |
| Kozak sequence | SEQ ID NO: 45 | 1256-1264 |
| CD64 signal peptide (SEQ | SEQ ID NO: 32 | 1262-1306 |
| ID NO: 23) | | |
| ACE2 EC domain | | 1307-3100 |
| CD28 hinge region | SEQ ID NO: 47 | 3101-3217 |
| CD28 transmembrane | SEQ ID NO: 48 | 3218-3298 |
| domain | | |
| CD28 (co-stimulatory | SEQ ID NO: 49 | 3299-3421 |
| domain) | | |
| CD3ζ cytoplasmic domain | SEQ ID NO: 50 | 3422-3763 |
| 3′UTR Mus Musculus | SEQ ID NO: 51 | 3770-3863 |
| hemoglobin alpha | | |
| 150 bp poly A tail | SEQ ID NO: 52 | 3870-4019 |
| SV40 poly A signal | SEQ ID NO: 53 | 4327-4461 |
| Puromycin resistance gene | SEQ ID NO: 54 | 4466-5065 |
| in reverse | | |
| Rep origin | SEQ ID NO: 13 | 7078-7656 |
| Ampicillin resistance gene | SEQ ID NO: 55 | 7736-8596 |
| RnnG terminator | SEQ ID NO: 56 | 8597-8729 |

TABLE 2
pNKW92

| | | pNKW92 |
|---|---|---|
| | | (CD64 VL-VH |
| | Nucleotide | B7H4 CAR) |
| | SEQ ID NO: | SEQ ID NO: 34 |
| Size | | 7709 bp |
| EF1a promoter | SEQ ID NO: 43 | 9-1190 |
| T7 promoter | SEQ ID NO: 44 | 1198-1216 |
| Kozak sequence | SEQ ID NO: 45 | 1256-1264 |
| CD64 signal peptide(SEQ | SEQ ID NO: 30 | 1262-1306 |
| ID NO: 23) | | |
| VH801 B7H4 | | 1307-1666 |
| Linker | | 1667-1723 |
| VL801 B7H4 | | 1724-2044 |
| CD28 hinge region | SEQ ID NO: 47 | 2045-2161 |
| CD28 transmembrane | SEQ ID NO: 48 | 2162-2242 |
| domain | | |
| CD28 co-stimulatory domain | SEQ ID NO: 49 | 2243-2365 |
| CD3ζ cytoplasmic domain | SEQ ID NO: 50 | 2366-2707 |
| 3′UTR Mus Musculus | SEQ ID NO: 51 | 2715-2808 |
| hemoglobin alpha | | |
| 150 bp poly A tail | SEQ ID NO: 52 | 2815-2967 |
| SV40 poly A signal | SEQ ID NO: 53 | 3272-3406 |
| Puromycin resistance gene in | SEQ ID NO: 54 | 3411-4010 |
| reverse | | |
| Rep origin | SEQ ID NO: 13 | 6023-6601 |
| Ampicillin resistance gene | SEQ ID NO: 55 | 6681-7541 |
| RnnG terminator | SEQ ID NO: 56 | 7542-7674 |

TABLE 3
pNKW93

| | | pNKW93 (IgGH-VH-VL |
|---|---|---|
| | Nucleotide | B7H4 CAR) |
| | SEQ ID NO: | SEQ ID NO: 35 |
| Size | | 7713 bp |
| EF1a promoter | SEQ ID NO: 43 | 9-1190 |
| T7 promoter | SEQ ID NO: 44 | 1198-1216 |
| Kozak sequence | SEQ ID NO: 45 | 1236-1244 |
| IgGHv signal peptide (SEQ | SEQ ID NO: 31 | 1242-1310 |
| ID NO: 46) | | |
| VH801 B7H4 | | 1311-1670 |
| Linker | | 1671-1727 |
| VH801 B7H4 | | 1728-2048 |
| CD28 hinge region | SEQ ID NO: 47 | 2049-2165 |
| CD28 transmembrane | SEQ ID NO: 48 | 2166-2246 |
| domain | | |
| CD28 co-stimulatory | SEQ ID NO: 49 | 2247-2369 |
| domain | | |
| CD3ζ cytoplasmic domain | SEQ ID NO: 50 | 2370-2711 |
| 3′UTR Mus Musculus | SEQ ID NO: 51 | 2719-2812 |
| hemoglobin alpha | | |
| 150 bp poly A tail | SEQ ID NO: 52 | 2819-2968 |
| SV40 poly A signal | SEQ ID NO: 53 | 3276-3410 |
| Puromycin resistance gene | SEQ ID NO: 54 | 3415-4014 |
| in reverse | | |
| Rep origin | SEQ ID NO: 13 | 6027-6605 |
| Amp resistance gene | SEQ ID NO: 55 | 6685-7545 |
| RnnG terminator | SEQ ID NO: 56 | 7546-7678 |

TABLE 4
pNKW88

| | | pNKW88 (IgGH-VL-VH |
|---|---|---|
| | Nucleotide | BCMA CAR) |
| | SEQ ID NO: | SEQ ID NO: 36 |
| Size | | 7692 bp |
| EF1a promoter | SEQ ID NO: 43 | 9-1190 |
| T7 promoter | SEQ ID NO: 44 | 1198-1216 |
| Kozak sequence | SEQ ID NO: 45 | 1236-1245 |
| IgGHv signal peptide (SEQ | SEQ ID NO: 28 | 1242-1310 |
| ID NO: 46) | | |
| BCMA VL | | 1311-1661 |
| Linker | | 1662-1706 |
| BCMA VH | | 1707-2027 |
| CD28 hinge region | SEQ ID NO: 47 | 2028-2144 |
| CD28 transmembrane | SEQ ID NO: 48 | 2145-2225 |
| domain | | |
| CD28 co-stimulatory | SEQ ID NO: 49 | 2226-2348 |
| domain | | |
| CD3ζ cytoplasmic domain | SEQ ID NO: 50 | 2349-2690 |
| 3′UTR Mus Musculus | SEQ ID NO: 51 | 2698-2791 |
| hemoglobin alpha | | |
| 150 bp poly A tail | SEQ ID NO: 52 | 2798-2947 |
| SV40 poly A signal | SEQ ID NO: 53 | 3255-3389 |
| Puromycin resistance gene | SEQ ID NO: 54 | 3394-3993 |
| in reverse | | |
| Rep origin | SEQ ID NO: 13 | 6006-6584 |
| Amp resistance gene | SEQ ID NO: 55 | 6664-7524 |
| RnnG terminator | SEQ ID NO: 56 | 7525-7657 |

TABLE 5
pNKW89

| | | pNKW89 (CD64 VL-VH |
|---|---|---|
| | Nucleotide | BCMA CAR) |
| | SEQ ID NO: | SEQ ID NO: 37 |
| Size | | 7688 bp |
| EF1a promoter | SEQ ID NO: 43 | 9-1190 |
| T7 promoter | SEQ ID NO: 44 | 1198-1216 |
| Kozak sequence | SEQ ID NO: 45 | 1256-1264 |
| CD64 signal peptide(SEQ | SEQ ID NO: 26 | 1262-1306 |
| ID NO: 23) | | |
| BCMA VL | | 1307-1657 |
| Linker | | 1658-1702 |
| BCMA VH | | 1703-2023 |
| CD28 hinge region | SEQ ID NO: 47 | 2024-2140 |
| CD28 transmembrane | SEQ ID NO: 48 | 2142-2221 |
| domain | | |
| CD28 co-stimulatory | SEQ ID NO: 49 | 2222-2344 |
| domain | | |
| CD3ζ cytoplasmic domain | SEQ ID NO: 50 | 2345-2686 |
| 3′UTR Mus Musculus | SEQ ID NO: 51 | 2694-2787 |
| hemoglobin alpha | | |
| 150 bp poly A tail | SEQ ID NO: 52 | 2794-2943 |
| SV40 poly A signal | SEQ ID NO: 53 | 3251-3385 |
| Puromycin resistance gene | SEQ ID NO: 54 | 3390-3989 |
| in reverse | | |
| Rep origin | SEQ ID NO: 13 | 6002-6580 |
| Ampicillin resistance gene | SEQ ID NO: 55 | 6660-7520 |
| RnnG terminator | SEQ ID NO: 56 | 7521-7653 |

TABLE 6
pNKW90

| | | pNKW90 (IgGH-VH-VL |
|---|---|---|
| | Nucleotide | BCMA CAR) |
| | SEQ ID NO: | SEQ ID NO: 38 |
| Size | | 7692 bp |
| EF1a promoter | SEQ ID NO: 43 | 9-1190 |
| T7 promoter | SEQ ID NO: 44 | 1198-1216 |
| Kozak sequence | SEQ ID NO: 45 | 1236-1245 |
| IgGHv signal peptide (SEQ | SEQ ID NO: 29 | 1242-1310 |
| ID NO: 46) | | |
| BCMA VH | | 1311-1631 |
| Linker | | 1632-1676 |
| BCMA VL | | 1677-2027 |
| CD28 hinge region | SEQ ID NO: 47 | 2028-2144 |
| CD28 transmembrane | SEQ ID NO: 48 | 2145-2225 |
| domain | | |
| CD28 co-stimulatory | SEQ ID NO: 49 | 2226-2348 |
| domain | | |
| CD3ζ cytoplasmic domain | SEQ ID NO: 50 | 2349-2690 |
| 3′UTR Mus Musculus | SEQ ID NO: 51 | 2698-2791 |
| hemoglobin alpha | | |
| 150 bp poly A tail | SEQ ID NO: 52 | 2798-2947 |
| SV40 poly A signal | SEQ ID NO: 53 | 3255-3389 |
| Puromycin resistance gene | SEQ ID NO: 54 | 3394-3993 |
| in reverse | | |
| Rep origin | SEQ ID NO: 13 | 6006-6584 |
| Amp resistance gene | SEQ ID NO: 55 | 6664-7524 |
| RnnG terminator | SEQ ID NO: 56 | 7525-7657 |

TABLE 7
pNKW91

| | | pNKW91 |
|---|---|---|
| | | (CD64 VH-VL |
| | Nucleotide | BCMA CAR) |
| | SEQ ID NO: | SEQ ID NO: 39 |
| Size | | 7688 bp |
| EF1a promoter | SEQ ID NO: 43 | 9-1190 |
| T7 promoter | SEQ ID NO: 44 | 1198-1216 |
| Kozak sequence | SEQ ID NO: 45 | 1256-1264 |
| CD64 signal peptide(SEQ ID | SEQ ID NO: 27 | 1262-1306 |
| NO: 23) | | |
| BCMA VH | | 1307-1627 |
| Linker | | 1628-1672 |
| BCMA VL | | 1673-2023 |
| CD28 hinge region | SEQ ID NO: 47 | 2024-2140 |
| CD28 transmembrane | SEQ ID NO: 48 | 2142-2221 |
| domain | | |
| CD28 co-stimulatory | SEQ ID NO: 49 | 2222-2344 |
| domain | | |
| CD3ζ cytoplasmic domain | SEQ ID NO: 50 | 2345-2686 |
| 3′UTR Mus Musculus | SEQ ID NO: 51 | 2694-2787 |
| hemoglobin alpha | | |
| 150 bp poly A tail | SEQ ID NO: 52 | 2794-2943 |
| SV40 poly A signal | SEQ ID NO: 53 | 3251-3385 |
| Puromycin resistance gene | SEQ ID NO: 54 | 3390-3989 |
| in reverse | | |
| Rep origin | SEQ ID NO: 13 | 6002-6580 |
| Ampicillin resistance gene | SEQ ID NO: 55 | 6660-7520 |
| RnnG terminator | SEQ ID NO: 56 | 7521-7653 |

TABLE 8
pNKW87

| | | pNKW87 (CD64-CD19 |
|---|---|---|
| | Nucleotide | CAR) |
| | SEQ ID NO: | SEQ ID NO: 40 |
| Size | | 7715 bp |
| EF1a promoter | SEQ ID NO: 43 | 9-1190 |
| T7 promoter | SEQ ID NO: 44 | 1198-1216 |
| Kozak sequence | SEQ ID NO: 45 | 1256-1264 |
| CD64 signal peptide(SEQ | SEQ ID NO: 24 | 1262-1306 |
| ID NO: 23) | | |
| FMC63 VL | | 1307-1630 |
| Linker | | 1631-1690 |
| FMC63 VH | | 1691-2050 |
| CD28 hinge region | SEQ ID NO: 47 | 2051-2167 |
| CD28 transmembrane | SEQ ID NO: 48 | 2168-2248 |
| domain | | |
| CD28 co-stimulatory | SEQ ID NO: 49 | 2249-2371 |
| domain | | |
| CD3ζ cytoplasmic domain | SEQ ID NO: 50 | 2372-2713 |
| 3′UTR Mus Musculus | SEQ ID NO: 51 | 2721-2814 |
| hemoglobin alpha | | |
| 150 bp poly A tail | SEQ ID NO: 52 | 2821-2970 |
| SV40 poly A signal | SEQ ID NO: 53 | 3278-3412 |
| Puromycin resistance gene | SEQ ID NO: 54 | 3417-4016 |
| in reverse | | |
| Rep origin | SEQ ID NO: 13 | 6029-6607 |
| Ampicilin resistance gene | SEQ ID NO: 55 | 6687-7547 |
| RnnG terminator | SEQ ID NO: 56 | 7548-7680 |

TABLE 9
pNKW59

| | | pNKW59 (IgGHv-CD19 |
|---|---|---|
| | Nucleotide | CAR) |
| | SEQ ID NO: | SEQ ID NO: 41 |
| Size | | 7739 bp |
| EF1a promoter | SEQ ID NO: 43 | 9-1190 |
| T7 promoter | SEQ ID NO: 44 | 1198-1216 |
| Kozak sequence | SEQ ID NO: 45 | 1256-1264 |
| IgGHv signal peptide (SEQ | SEQ ID NO: 25 | 1262-1318 |
| ID NO: 46) | | |
| FMC63 VL | | 1331-1654 |
| Linker | | 1655-1714 |
| FMC63 VH | | 1715-2074 |
| CD28 hinge region | SEQ ID NO: 47 | 2075-2191 |
| CD28 transmembrane | SEQ ID NO: 48 | 2192-2272 |
| domain | | |
| CD28 co-stimulatory | SEQ ID NO: 49 | 2273-2395 |
| domain | | |
| CD3ζ cytoplasmic domain | SEQ ID NO: 50 | 2396-2737 |
| 3′UTR Mus Musculus | SEQ ID NO: 51 | 2745-2828 |
| hemoglobin alpha | | |
| 150 bp poly A tail | SEQ ID NO: 52 | 2845-2994 |
| SV40 poly A signal | SEQ ID NO: 53 | 3302-3436 |
| Puromycin resistance gene | SEQ ID NO: 54 | 3441-4040 |
| in reverse | | |
| Rep origin | SEQ ID NO: 13 | 6053-6631 |
| Ampicilin resistance gene | SEQ ID NO: 55 | 6711-7571 |
| RnnG terminator | SEQ ID NO: 56 | 7572-7704 |

TABLE 10
pNKW95 (CD30 alpha)

| | | pNKW95 (CD64 VL-VH |
|---|---|---|
| | Nucleotide | CD30α CAR) |
| | SEQ ID NO: | SEQ ID NO: 42 |
| Size | | 7739 bp |
| EF1a promoter | SEQ ID NO: 43 | 9-1190 |
| T7 promoter | SEQ ID NO: 44 | 1198-1216 |
| Kozak sequence | SEQ ID NO: 45 | 1256-1264 |
| CD64 signal peptide (SEQ | SEQ ID NO: 62 | 1262-1306 |
| ID NO: 23) | | |
| 52-38 SLH28z VL CD30 | | 1307-1627 |
| Linker | | 1628-1702 |
| 52-38 SLH28z VH CD30 | | 1703-2074 |
| CD28 hinge region | SEQ ID NO: 47 | 2075-2191 |
| CD28 transmembrane | SEQ ID NO: 48 | 2192-2272 |
| domain | | |
| CD28 co-stimulatory | SEQ ID NO: 49 | 2273-2395 |
| domain | | |
| CD3ζ cytoplasmic domain | SEQ ID NO: 50 | 2396-2737 |
| 3′UTR Mus Musculus | SEQ ID NO: 51 | 2745-2838 |
| hemoglobin alpha | | |
| 150 bp poly A tail | SEQ ID NO: 52 | 2845-2997 |
| SV40 poly A signal | SEQ ID NO: 53 | 3302-3436 |
| Puromycin resistance gene | SEQ ID NO: 54 | 3411-4040 |
| in reverse | | |
| Rep origin | | 6052-6631 |
| Ampicillin resistance gene | SEQ ID NO: 55 | 6711-7571 |
| RnnG terminator | SEQ ID NO: 56 | 7572-7704 |

### Example 2

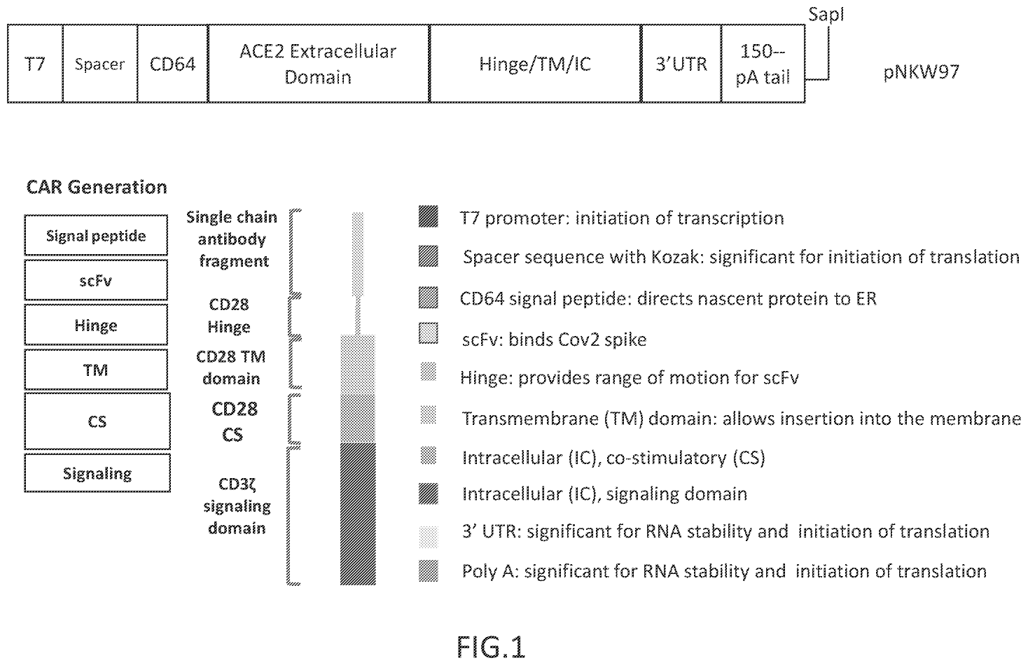

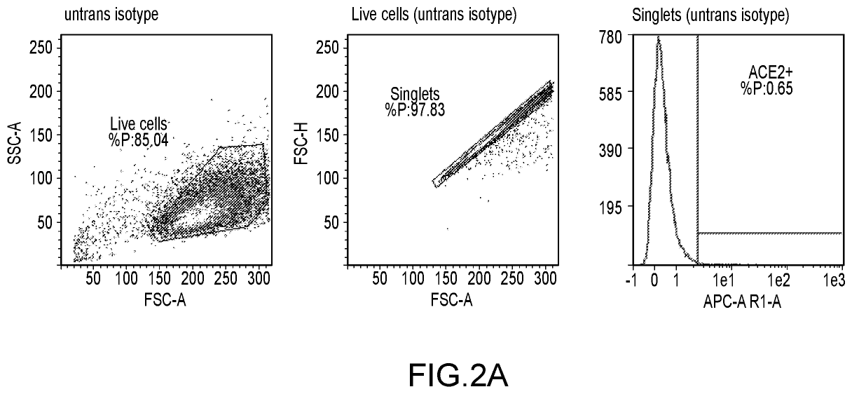

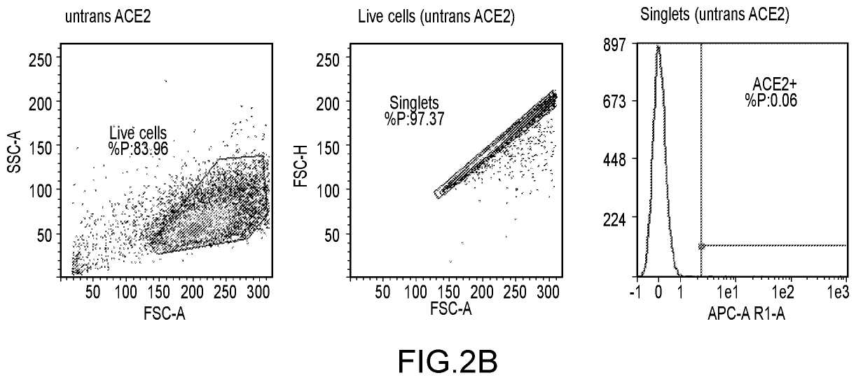

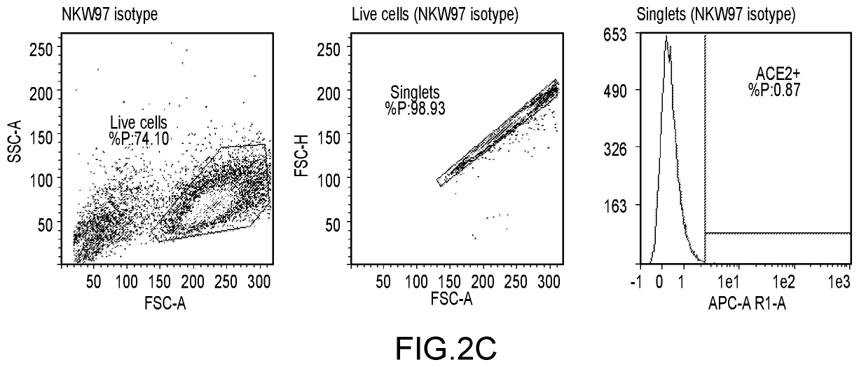

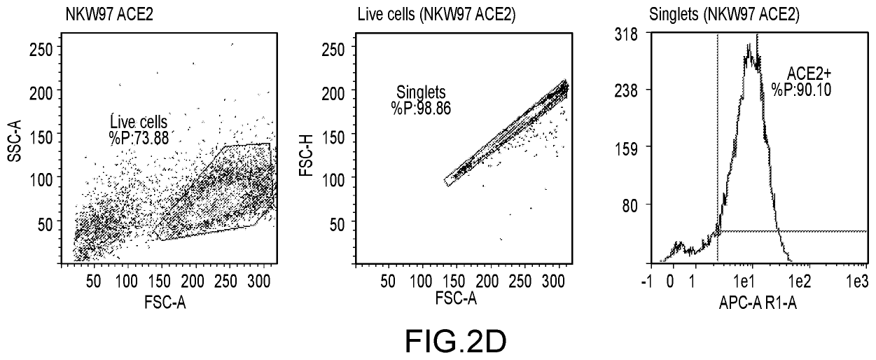

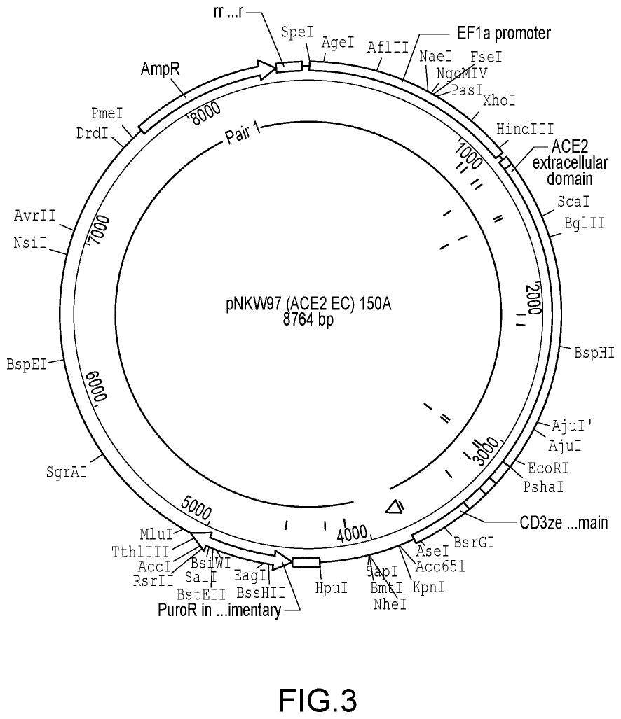

This example shows the results with ACE pNKW97 (FIGS. 1, 2A-2D) The extracellular domain of the ACE2 protein was cloned into a vector containing the hinge, TM and co-stimulatory domains of CD28 as well a CD3ζ signaling domain and a 150p-A (FIG. 1). The construct was digested with SapI and the linearized DNA was used as a template for mRNA generation. PBNK cells were electroporated with the pNKW97 mRNA. After overnight recovery, ACE2 expression was detected using flow cytometry and a conjugated anti-ACE2 antibody. An isotype control antibody was used to confirm specificity of the ACE2 antibody (FIG. 2A, 2C). A no-transfection control (FIG. 2B) shows no endogenous expression of ACE2 in CENK cells. Electroporated CENK cells, however, show >90% ACE2 CAR expression (FIG. 2D). The vector encoding the ACE2 CAR is shown in FIG. 3.

### Example 3

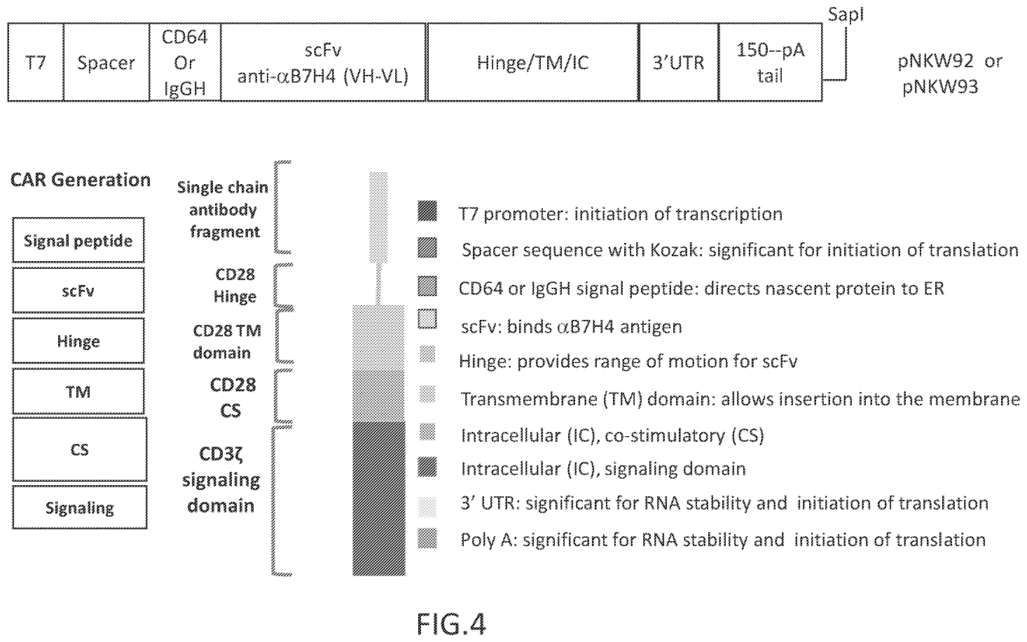

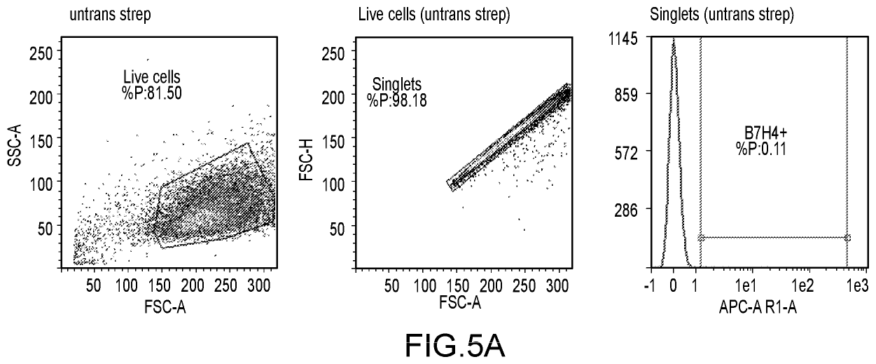

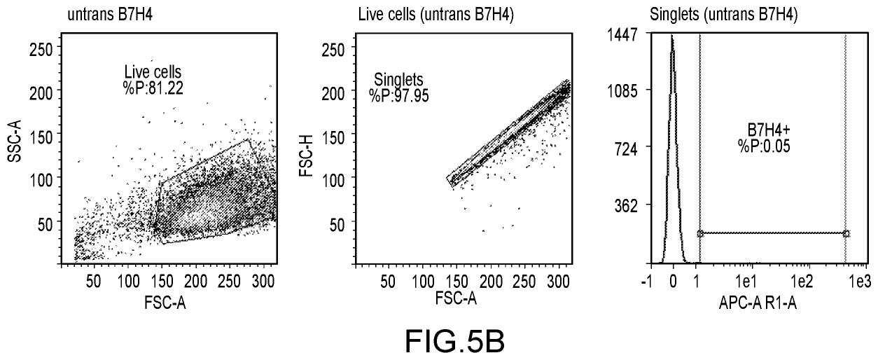

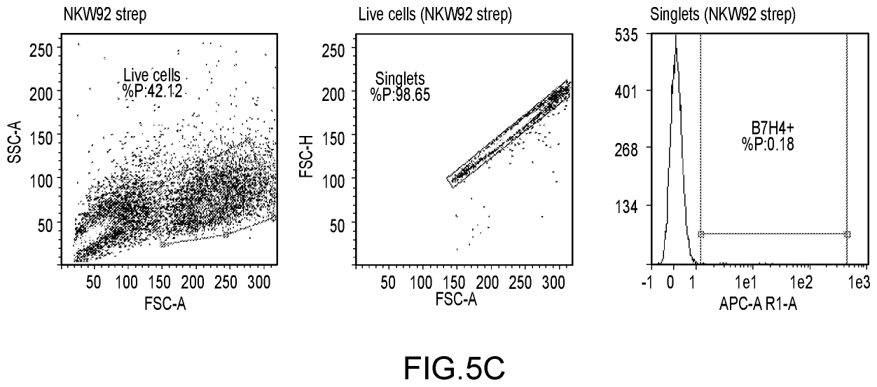

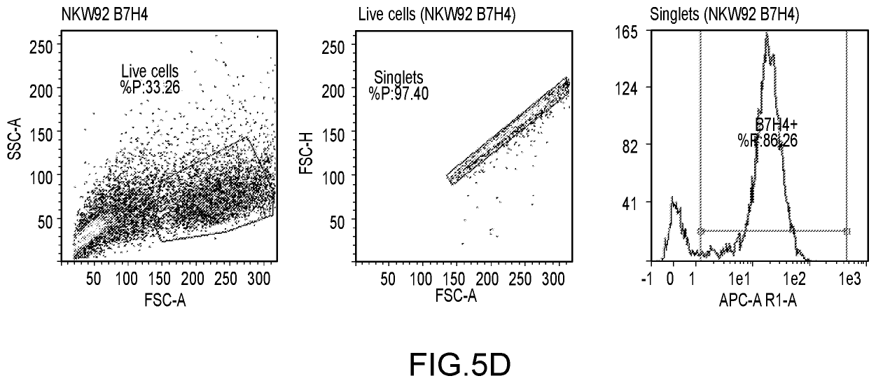

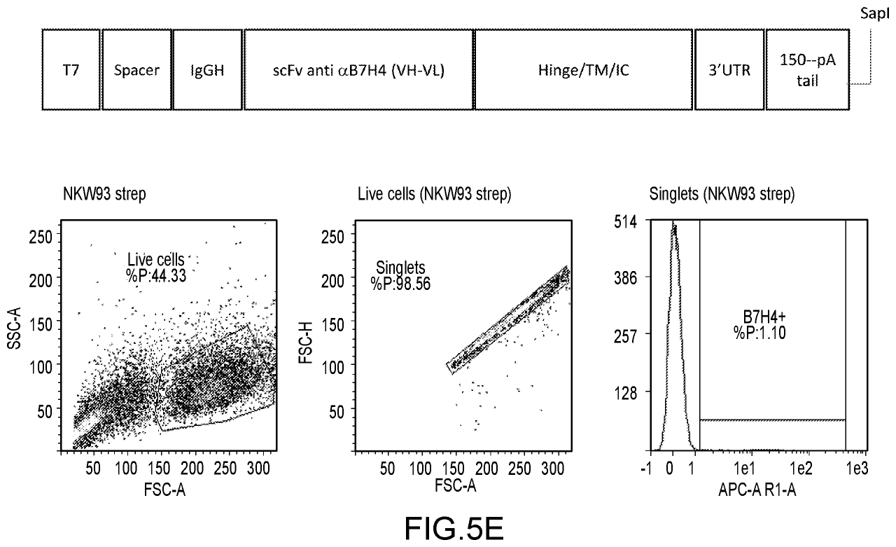

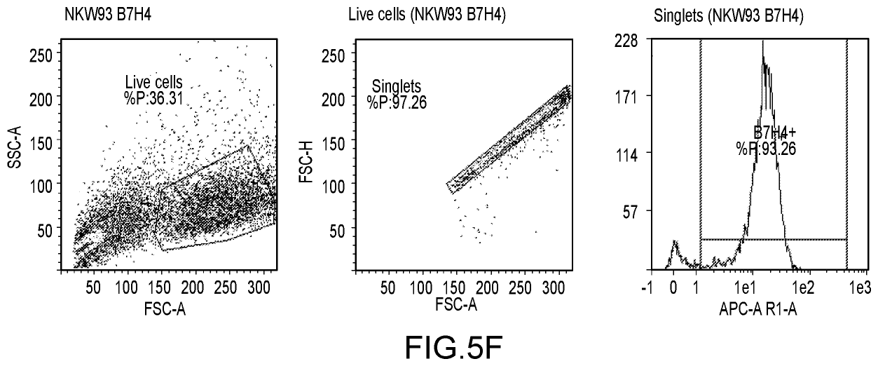

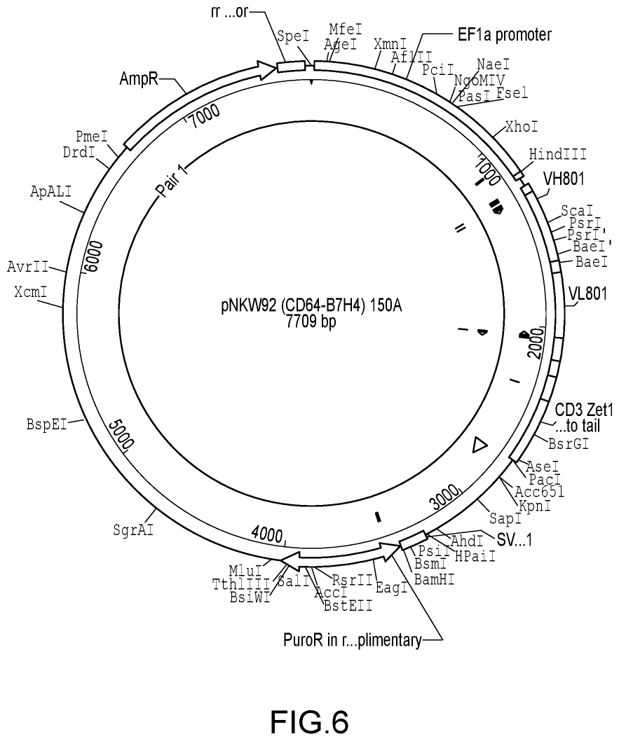

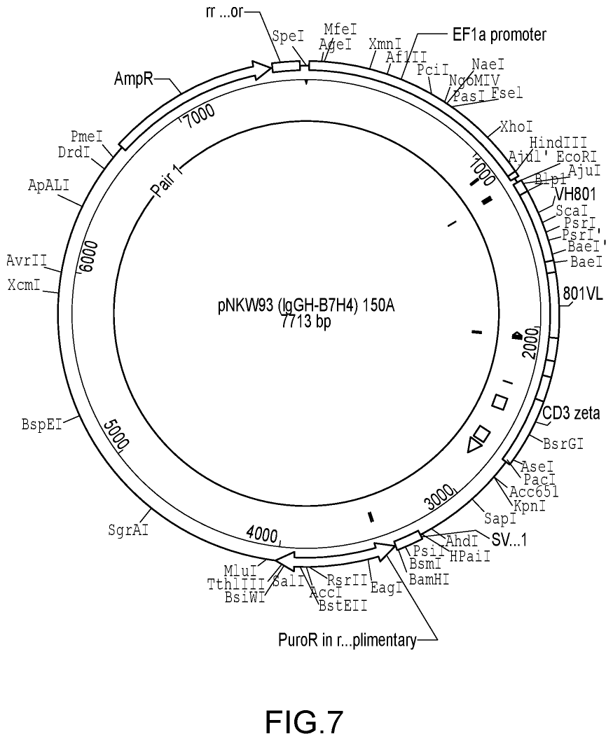

This example shows the results with pNKW92-93 (FIGS. 4, 5A-5F). A CAR molecule targeting the B7H4 antigen preceded by a CD64 or an IgGH signal peptide was designed as shown in FIG. 4. The CAR DNA was linearized with SapI and served as a template for in vitro transcription of mRNA. Next CENK cells were electroporated with the mRNA. After overnight recovery, expression of the B7H4 CAR was detected using flow cytometry and a biotinylated B7H4 followed by streptavidin-APC. As a control for the specificity of the detection reagent, a streptavidin-APC only staining was used for each sample. As shown in FIGS. 5A and 5B, the no transfection control did not express B7H4 CAR. On the other hand, both pNKW92 (CD64 signal peptide) and pNKW93 (IgGHv signal peptide) (FIGS. 5C, 5D and 5E, 5F respectively) showed high expression of the B7H4 CAR. The vectors encoding the B7H4 CAR are shown in FIGS. 6 and 7.

### Example 4

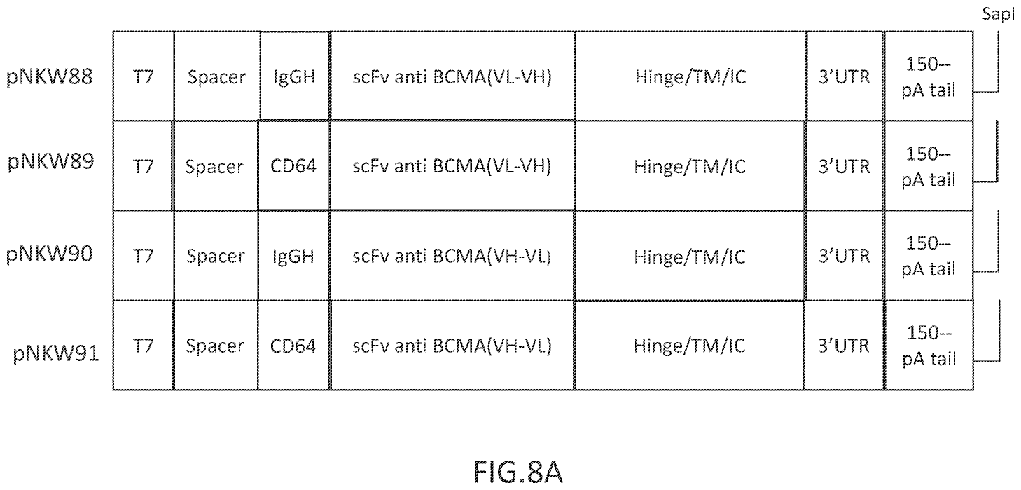

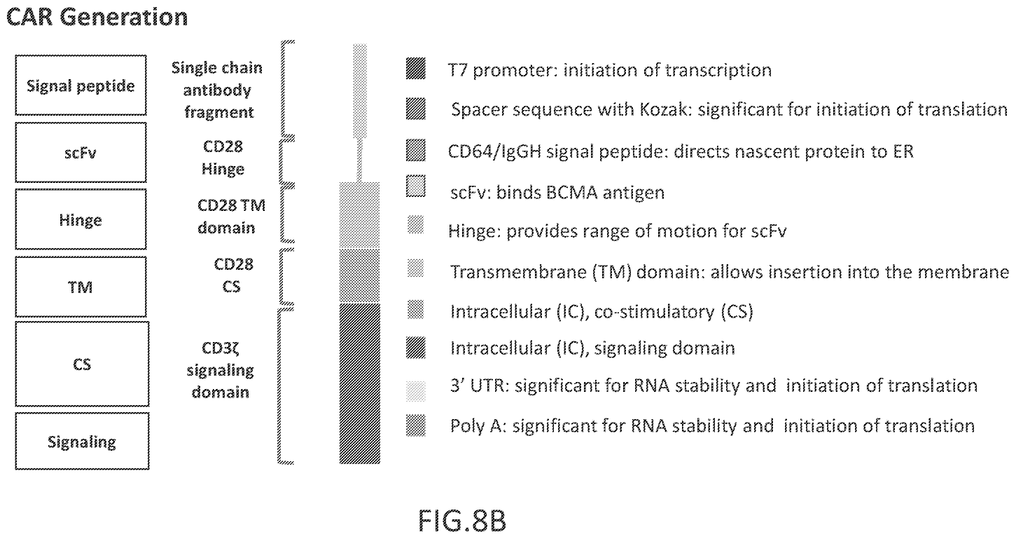

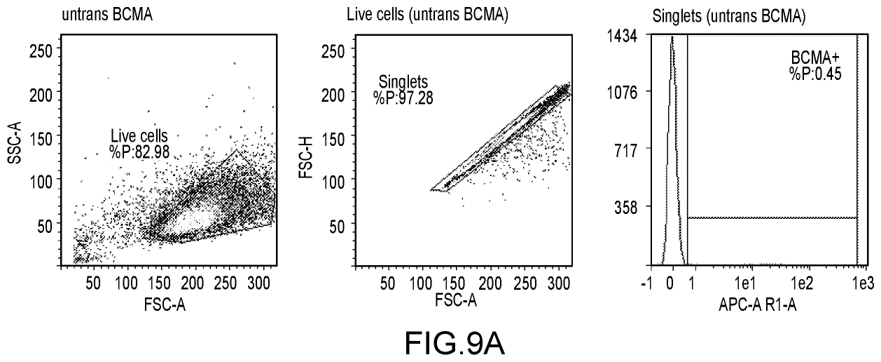

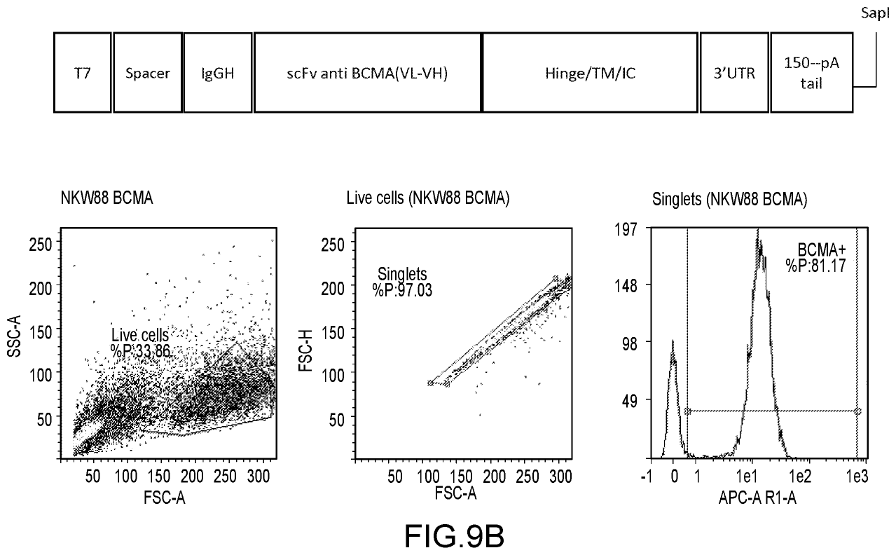

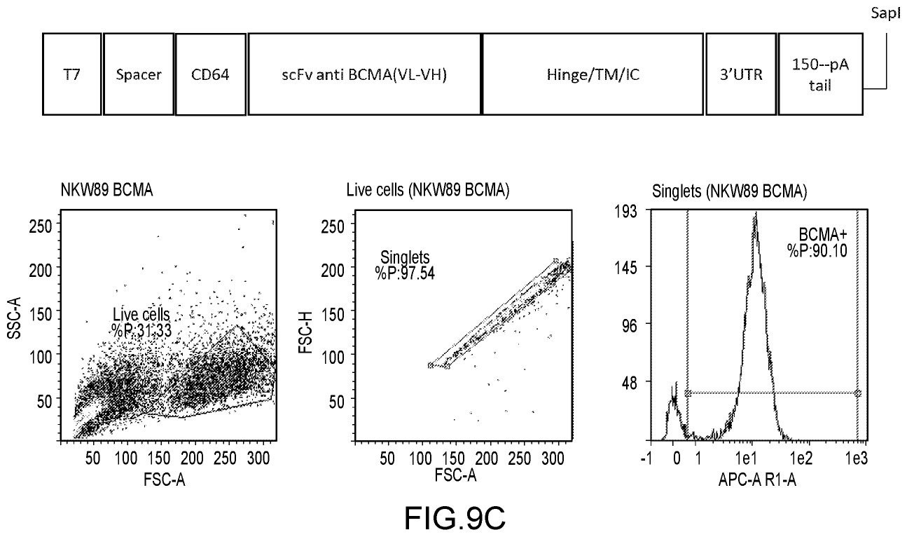

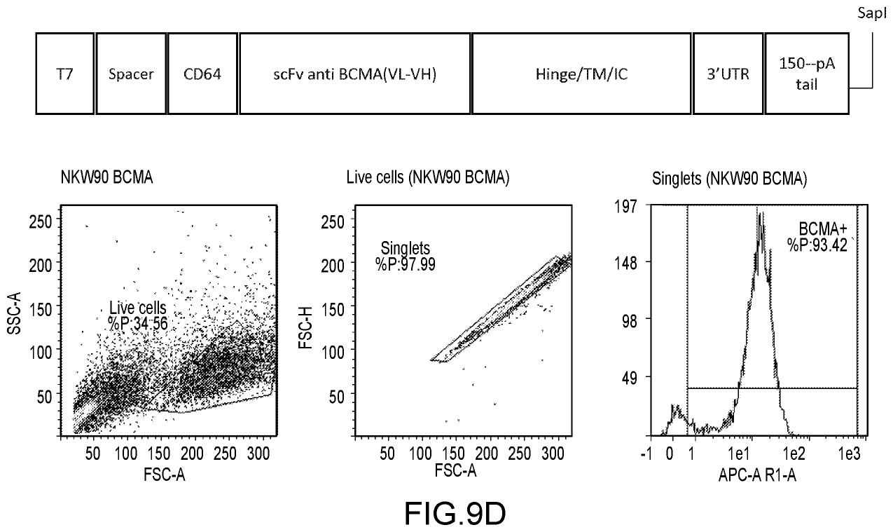

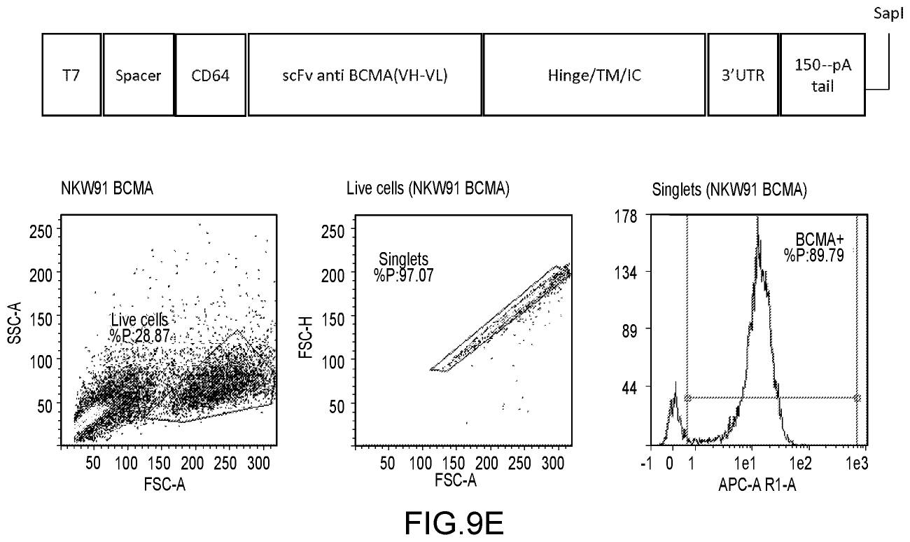

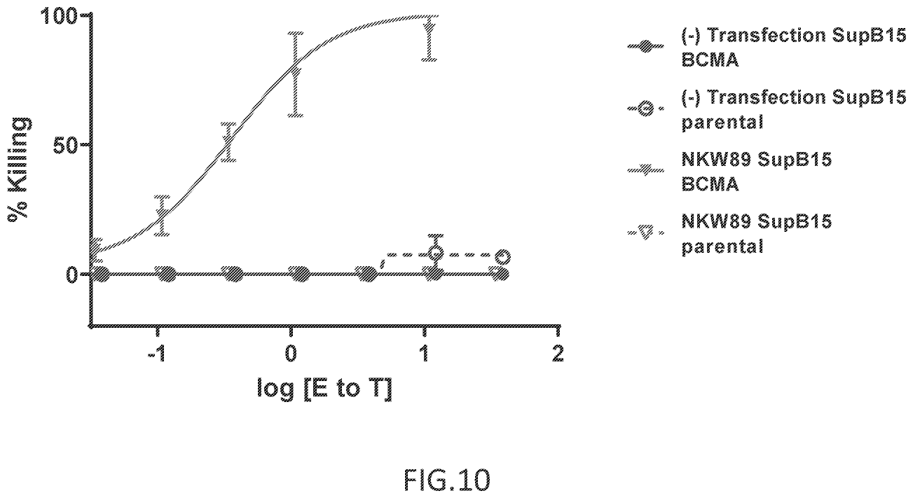

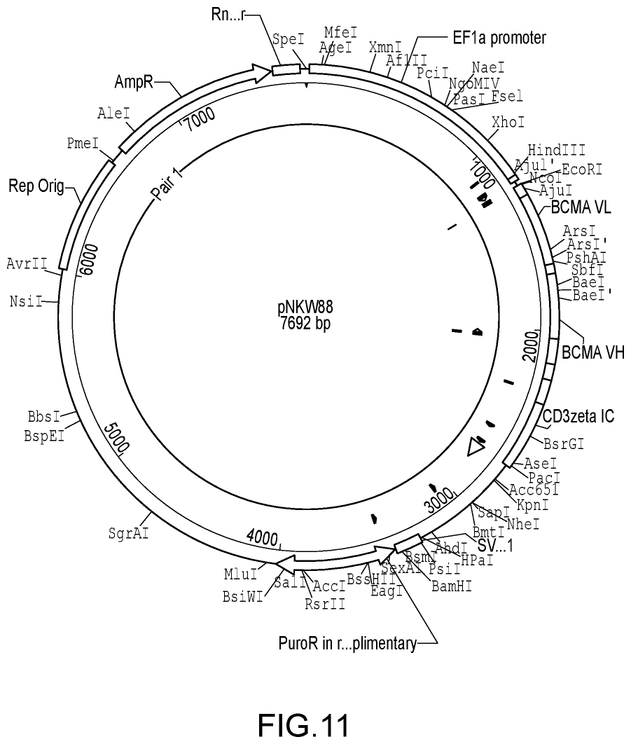

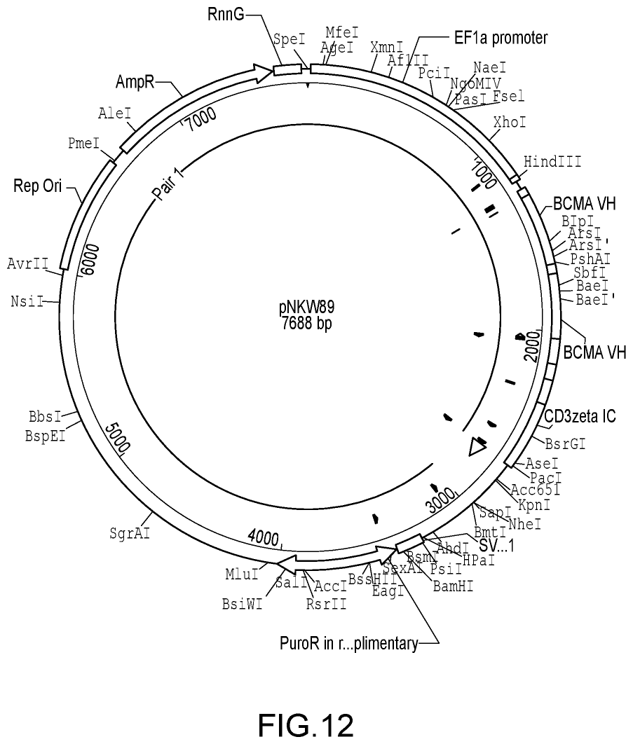

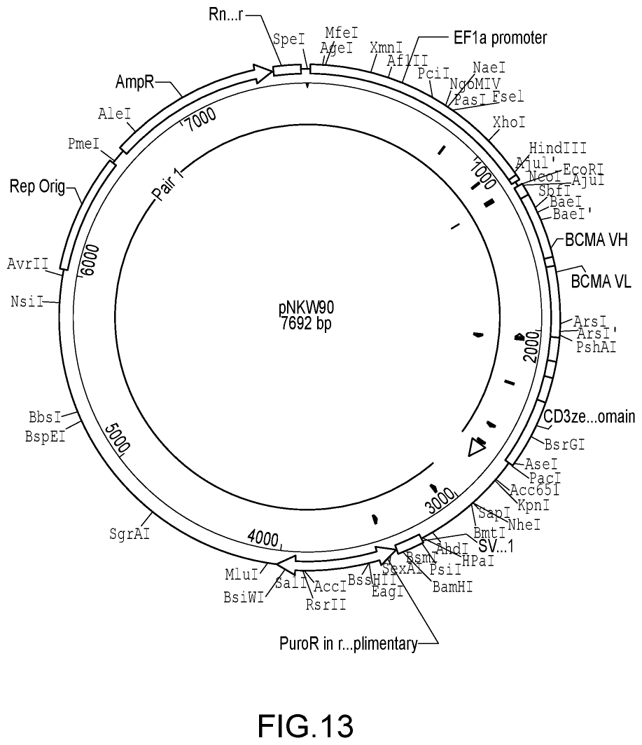

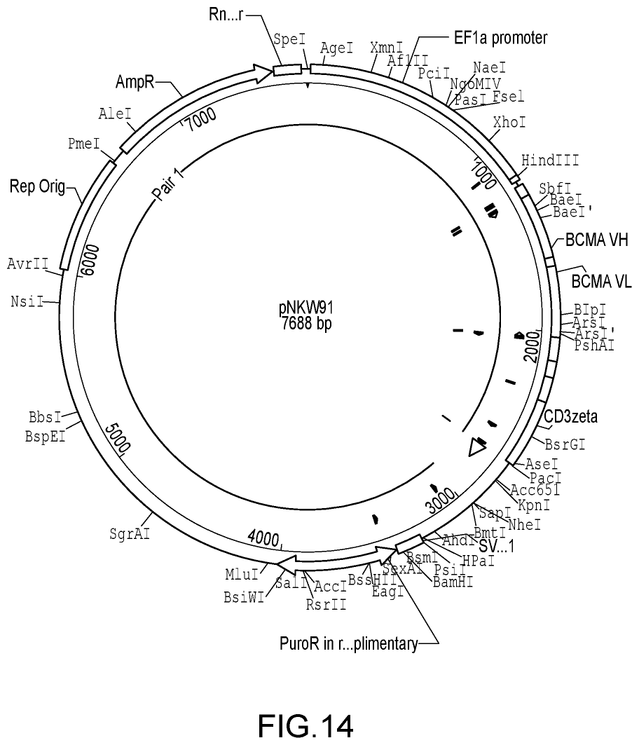

This example shows the results with pNKW88-91 (FIGS. 8A, B, 9A-9E, 10-14). Four CAR molecules targeting the BCMA antigen were designed as shown in FIGS. 8A and 8B. The CAR DNA was linearized with SapI and served as a template for in vitro transcription of mRNA. Next, CENK cells were electroporated with the mRNA. After overnight recovery, expression of BCMA CAR was detected using flow cytometry and a biotinylated BCMA followed by streptavidin-APC. As a control for the specificity of the detection reagent, a streptavidin-APC only staining was used for each sample. As shown in FIG. 9A, the no transfection control did not express the BCMA CAR. On the other hand, all 4 different constructs (FIGS. 9B-9E) showed high levels of BCMA CAR. The constructs were tested for cytotoxic activity against a SUP-B15 cell line stably expressing BCMA (data not shown for all constructs). In FIG. 10, one CAR construct (pNKW89) is shown to specifically lyse the BCMA expressing target cell line while it had no cytotoxic activity on the BCMA-negative parental cell line. The no transfection control has no cytotoxicity against either parental SUP-B15 or the BCMA expressing SUP-B15 cell line. The vectors encoding the BCMA CAR are shown in FIGS. 11-14.

### Example 5

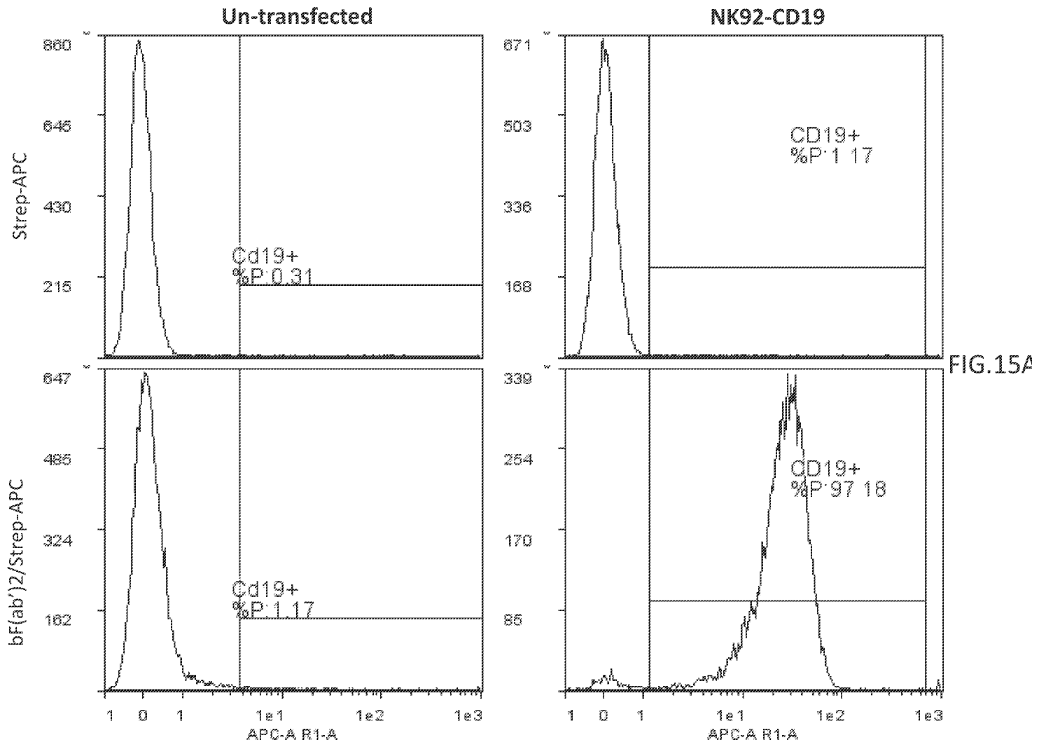

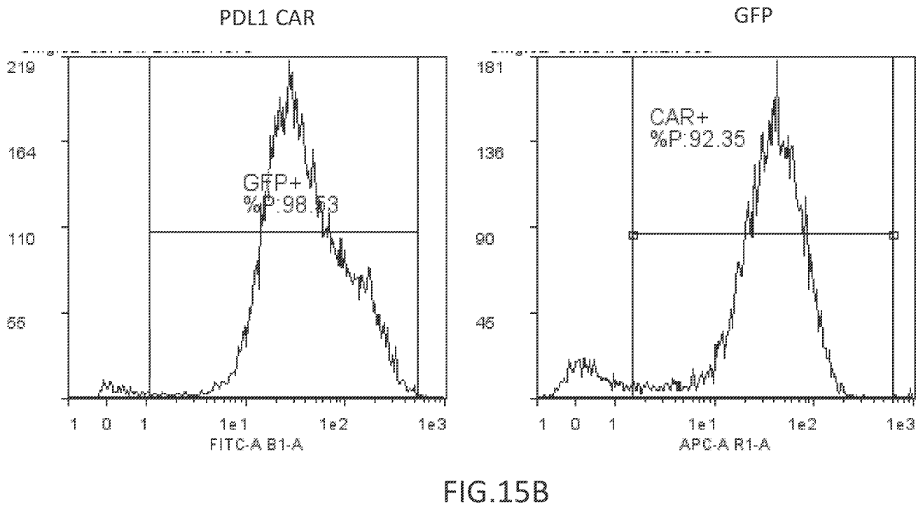

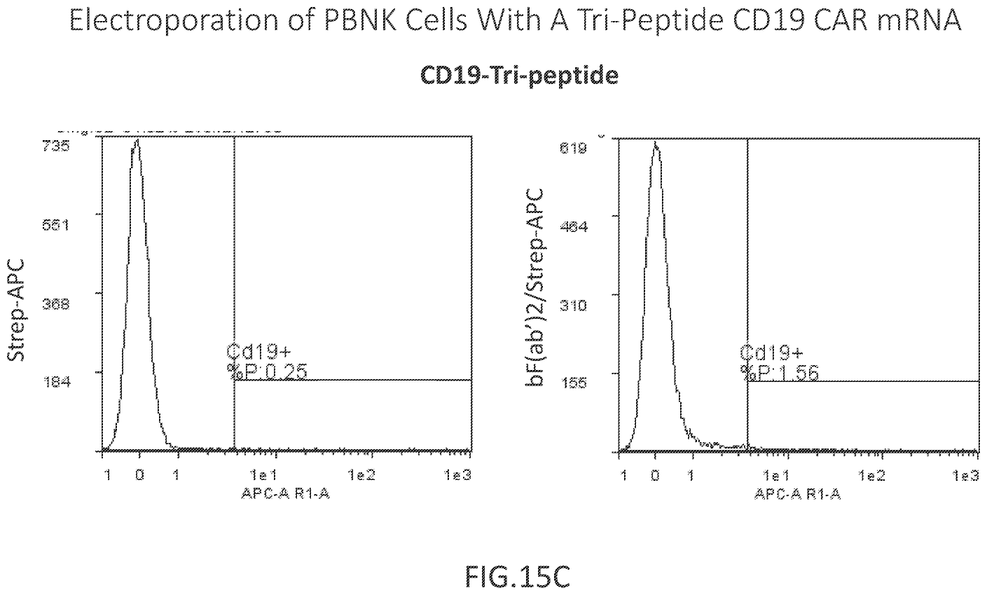

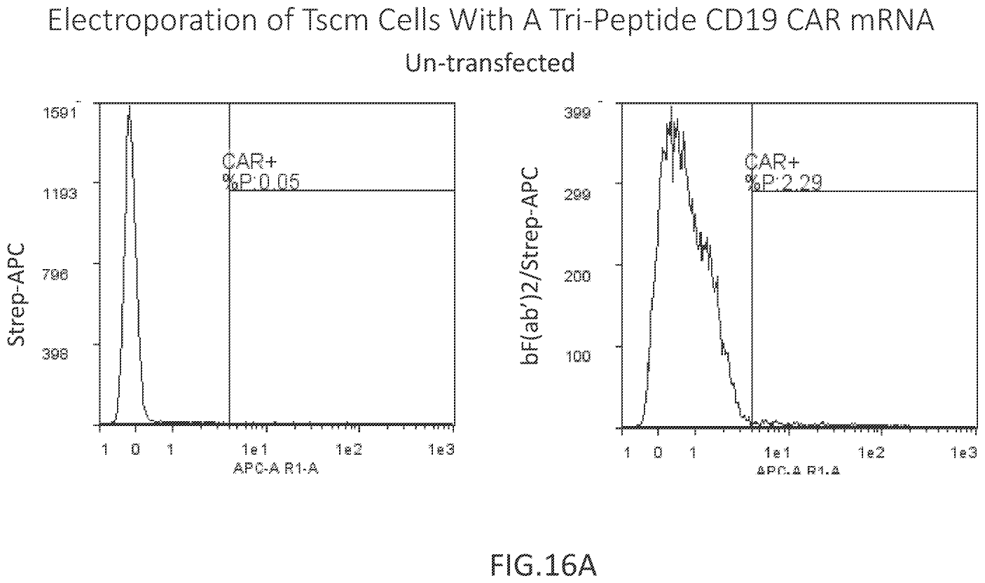

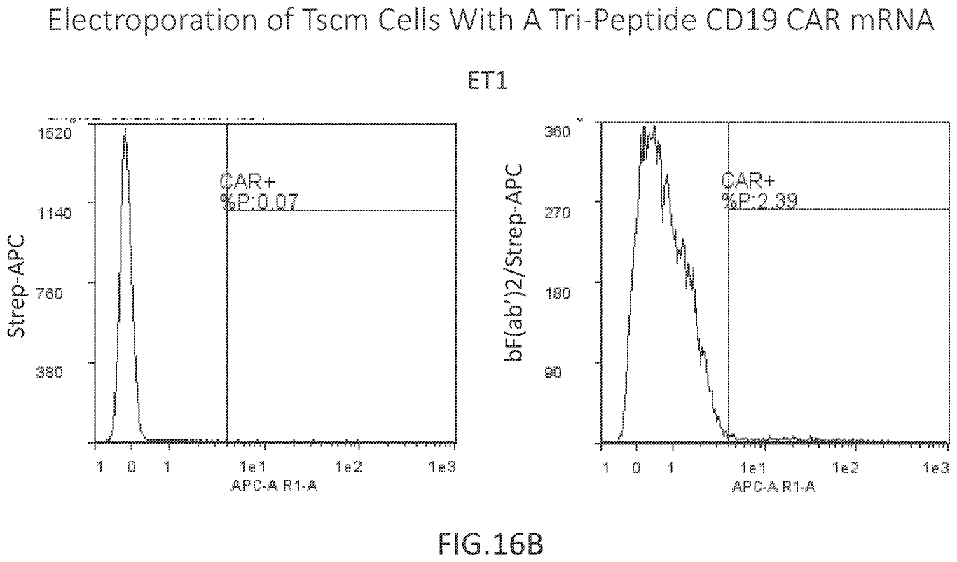

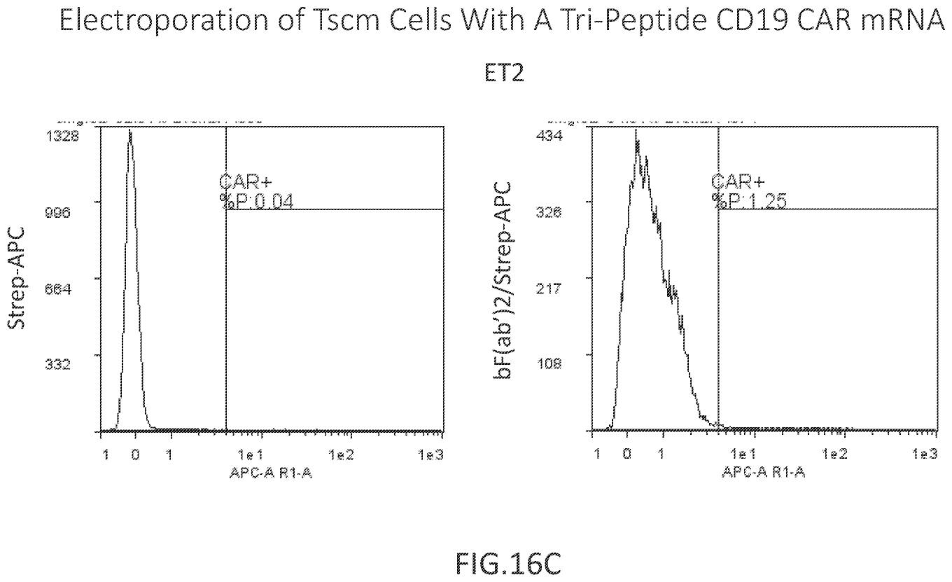

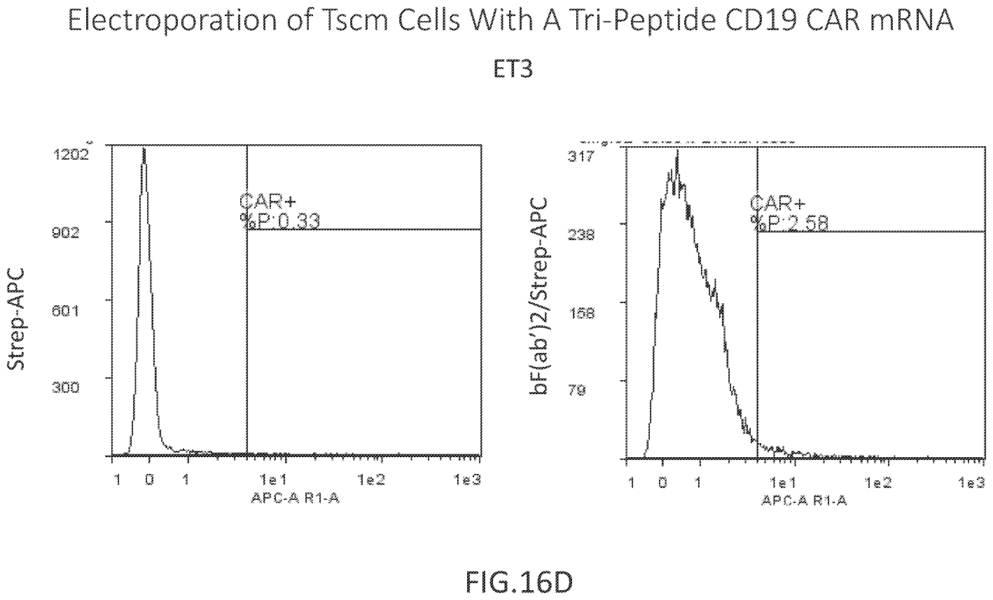

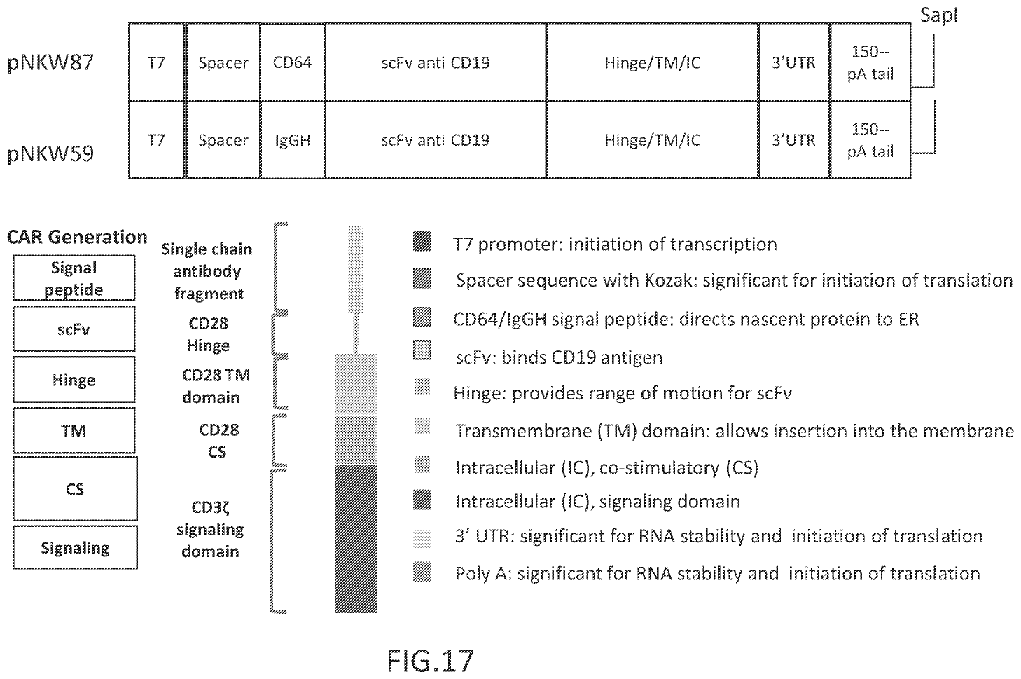

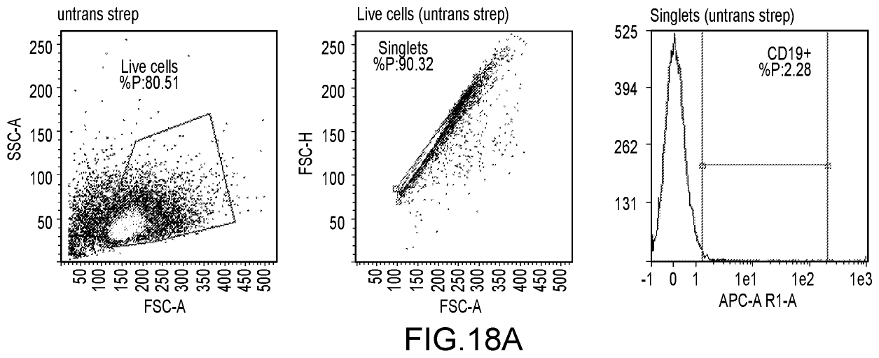

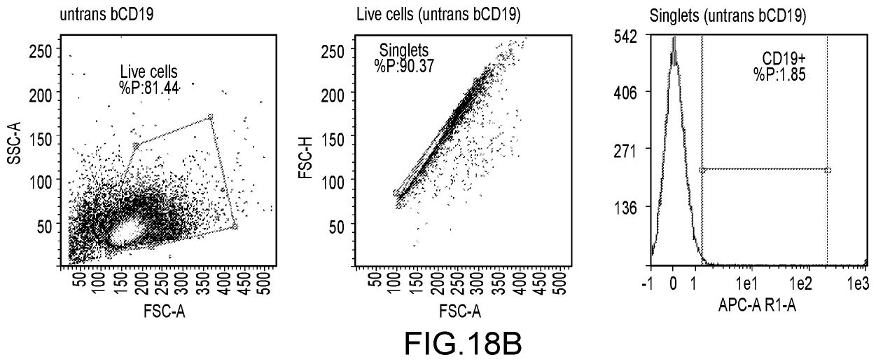

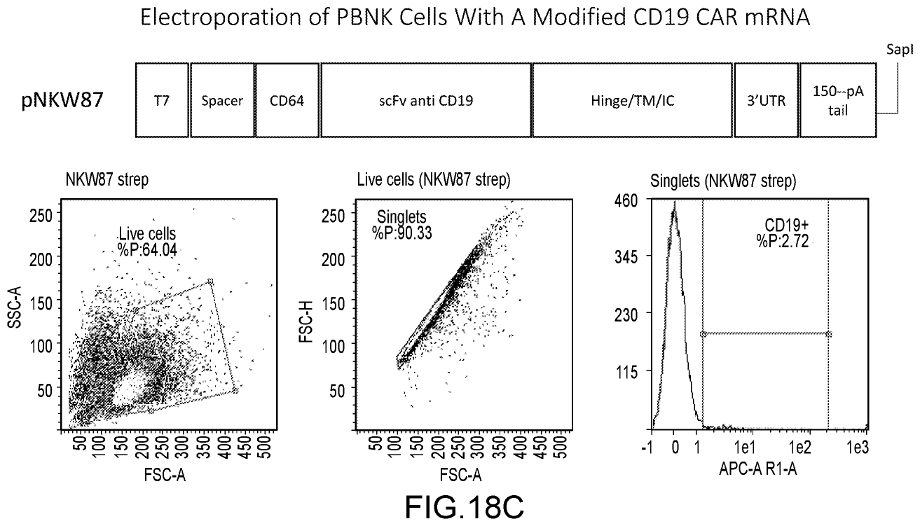

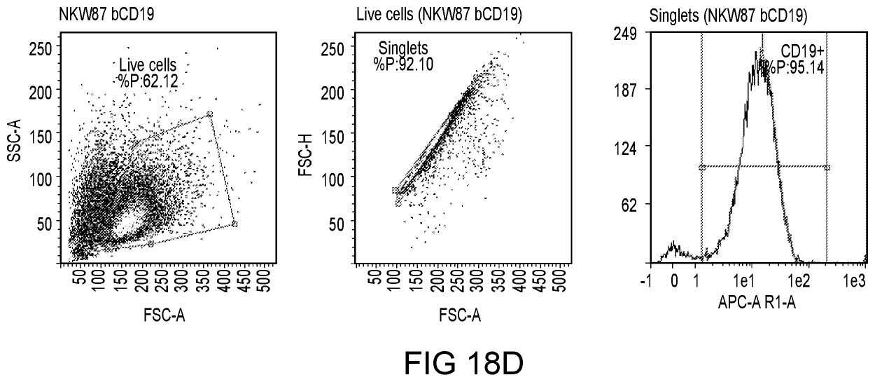

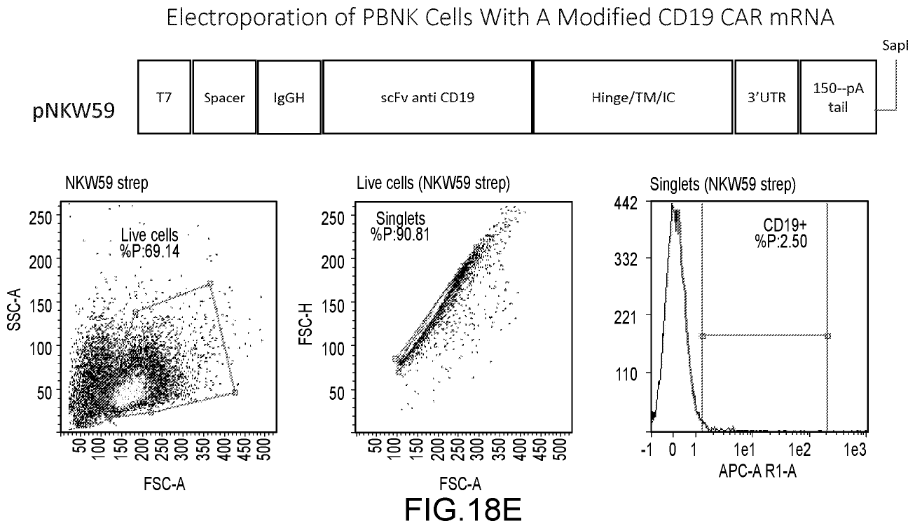

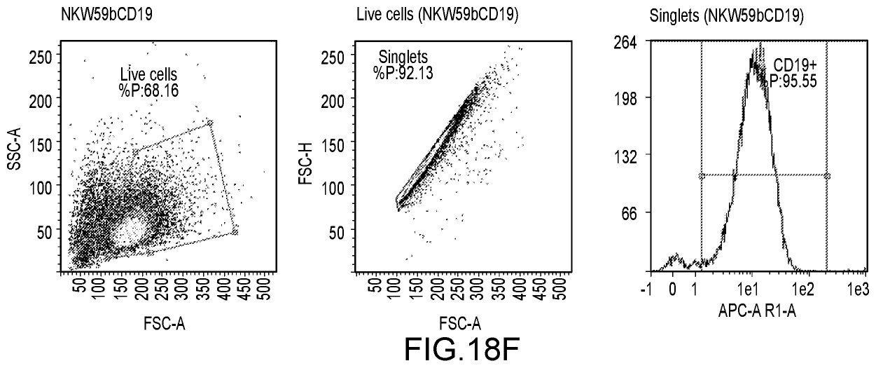

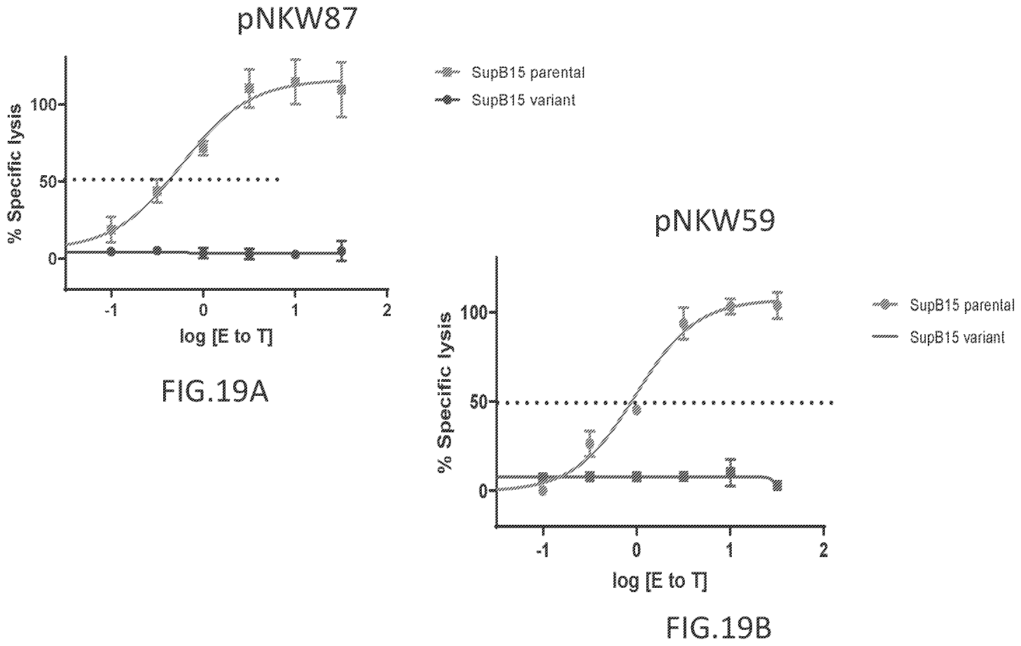

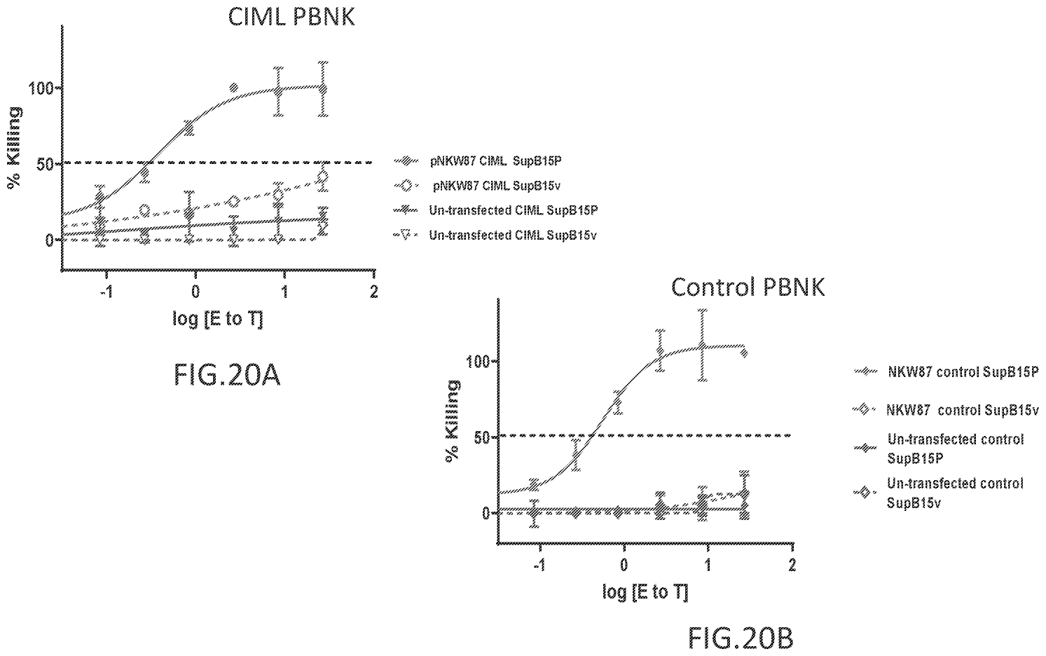

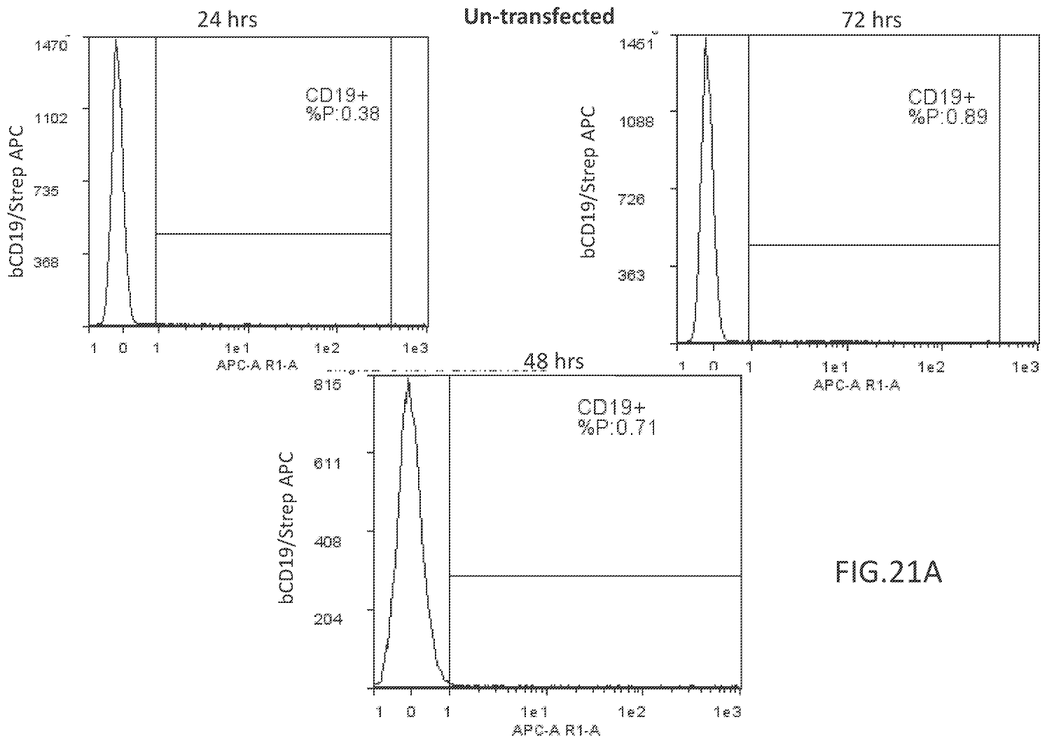

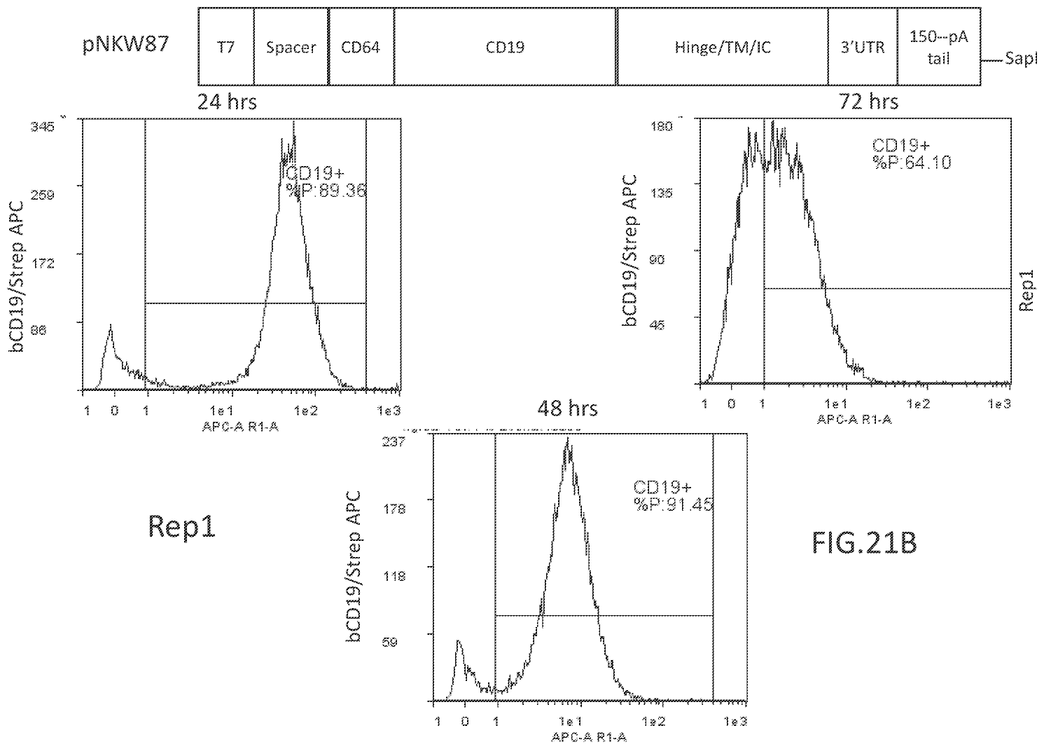

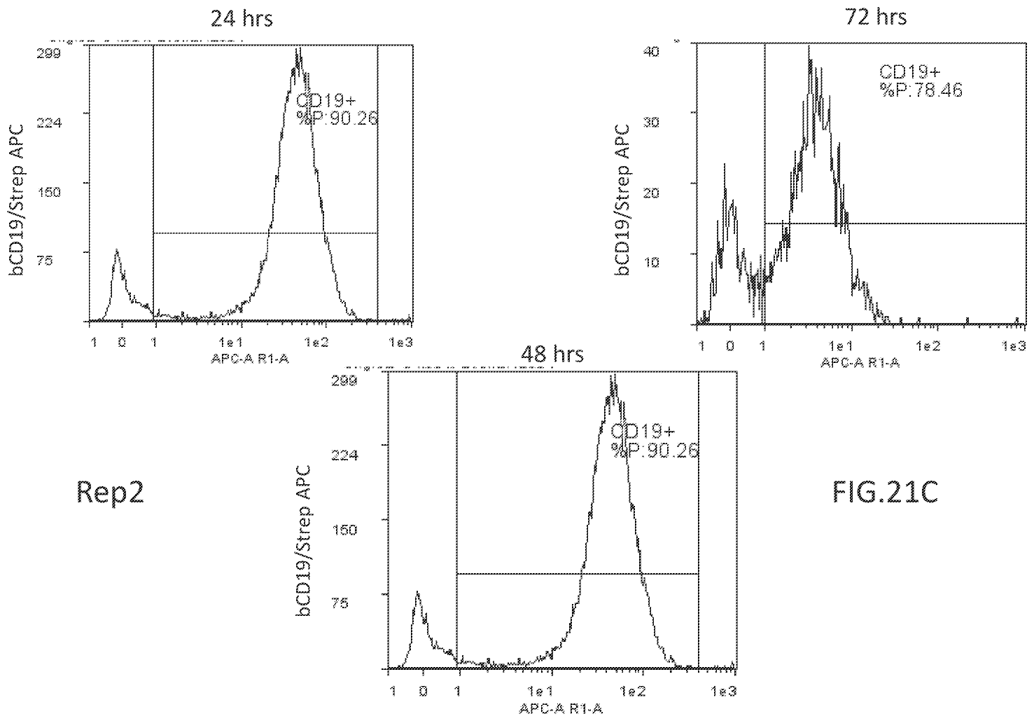

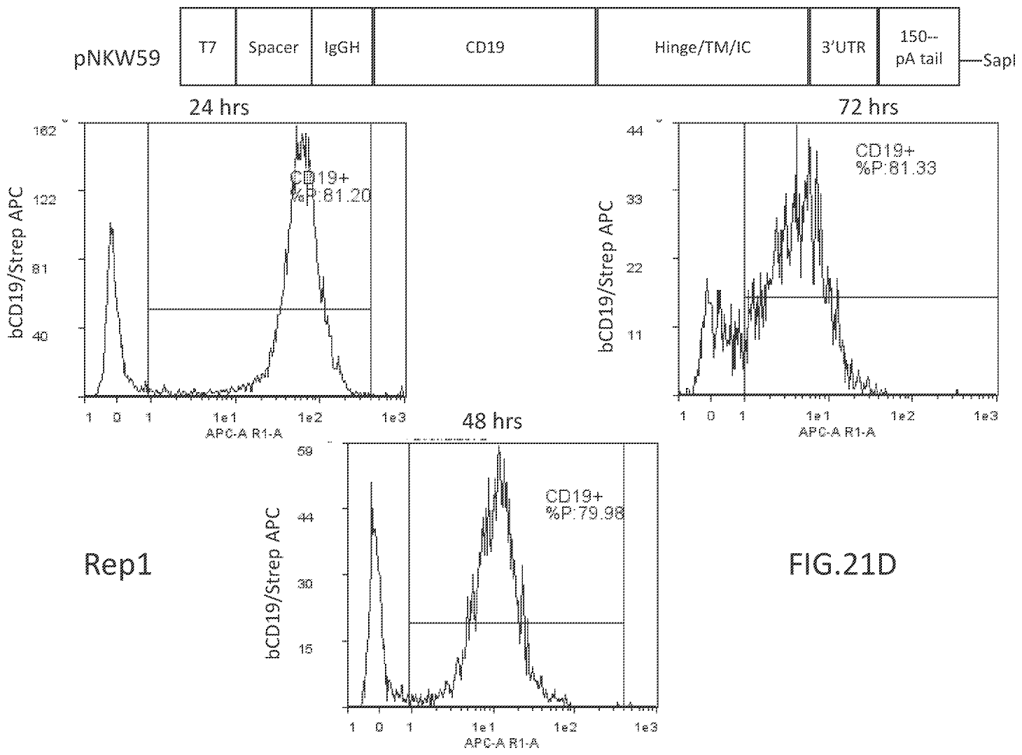

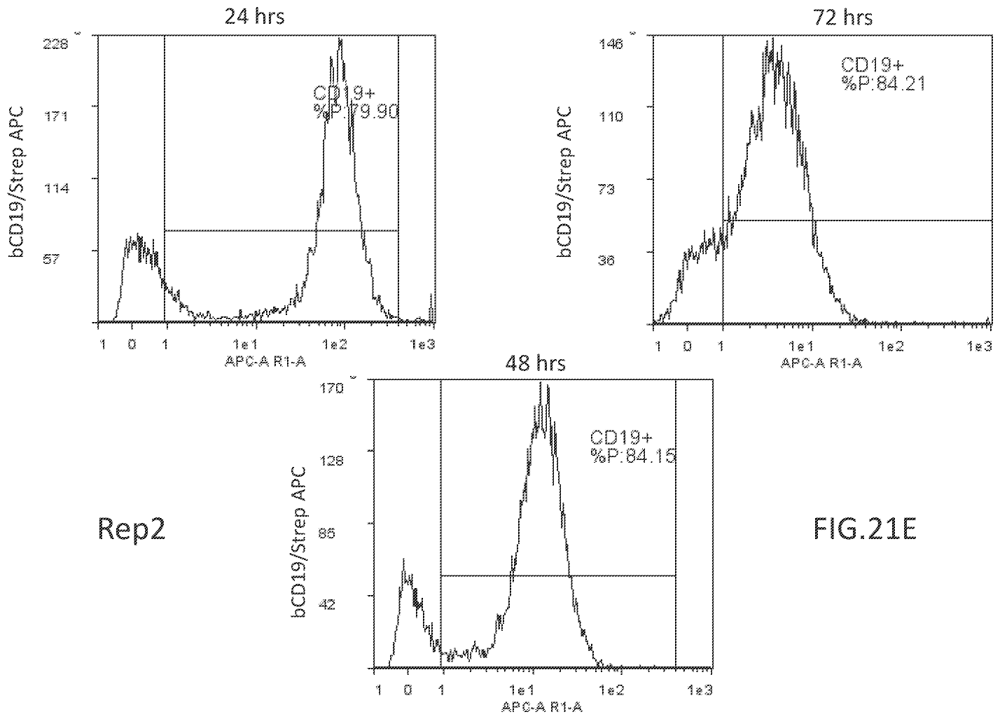

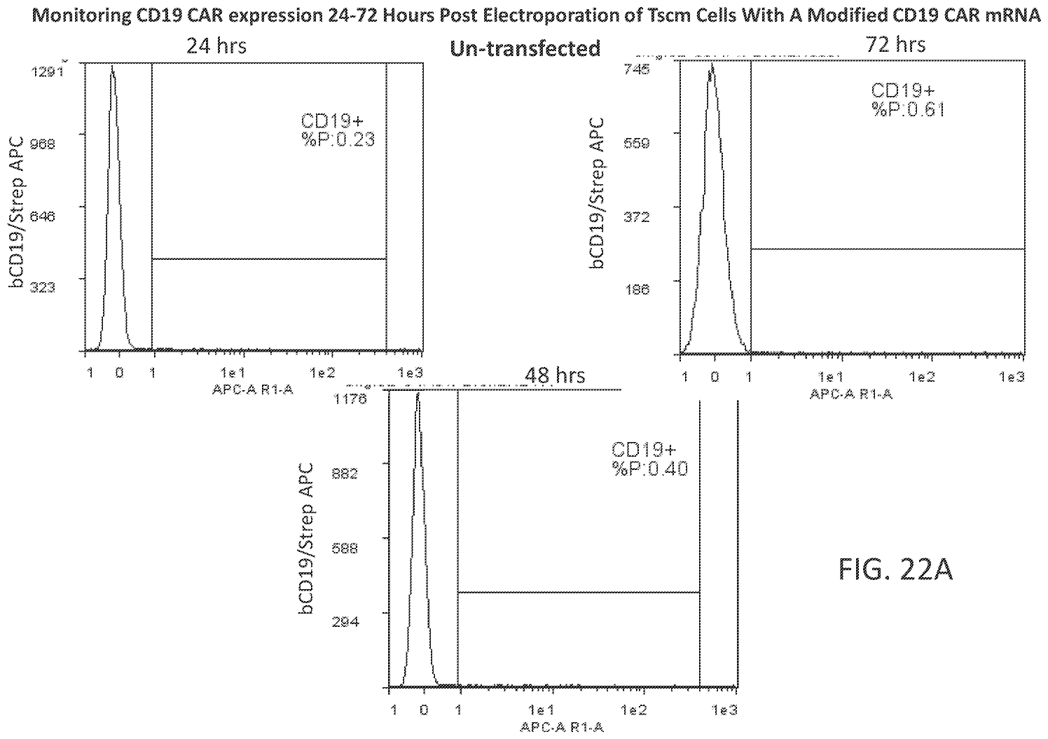

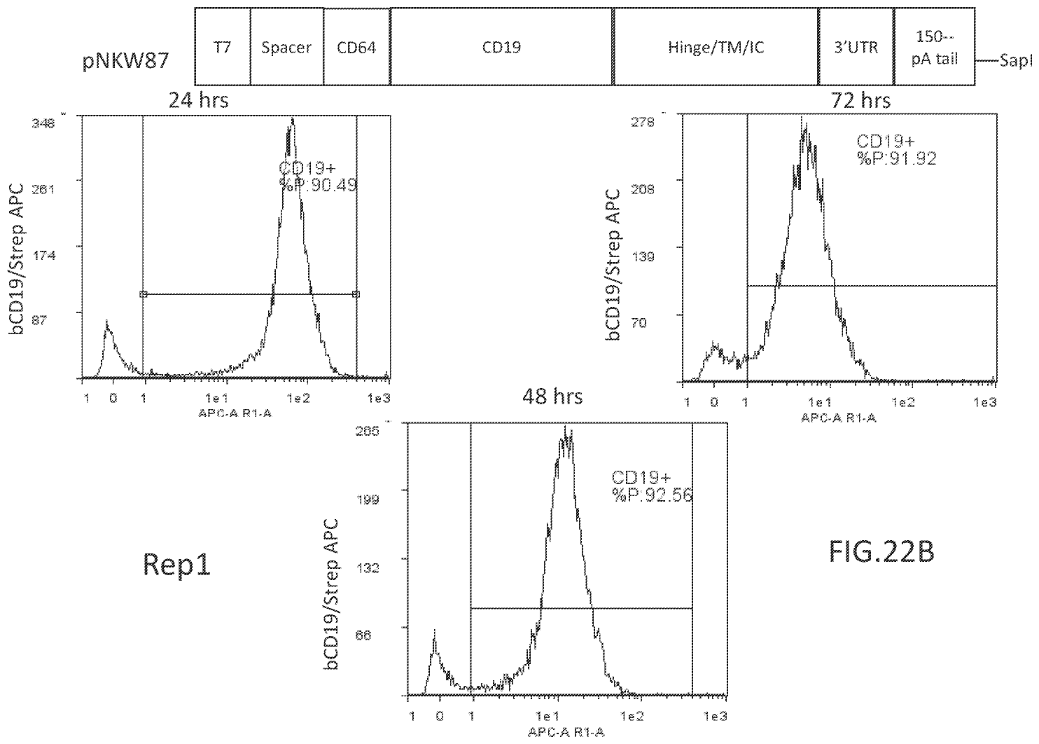

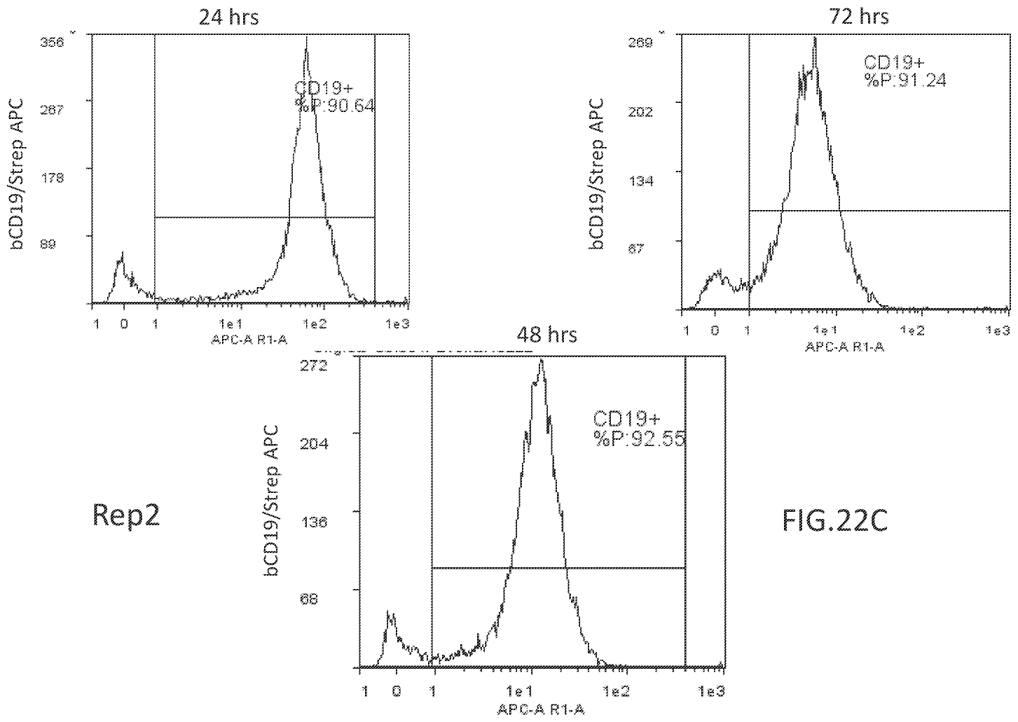

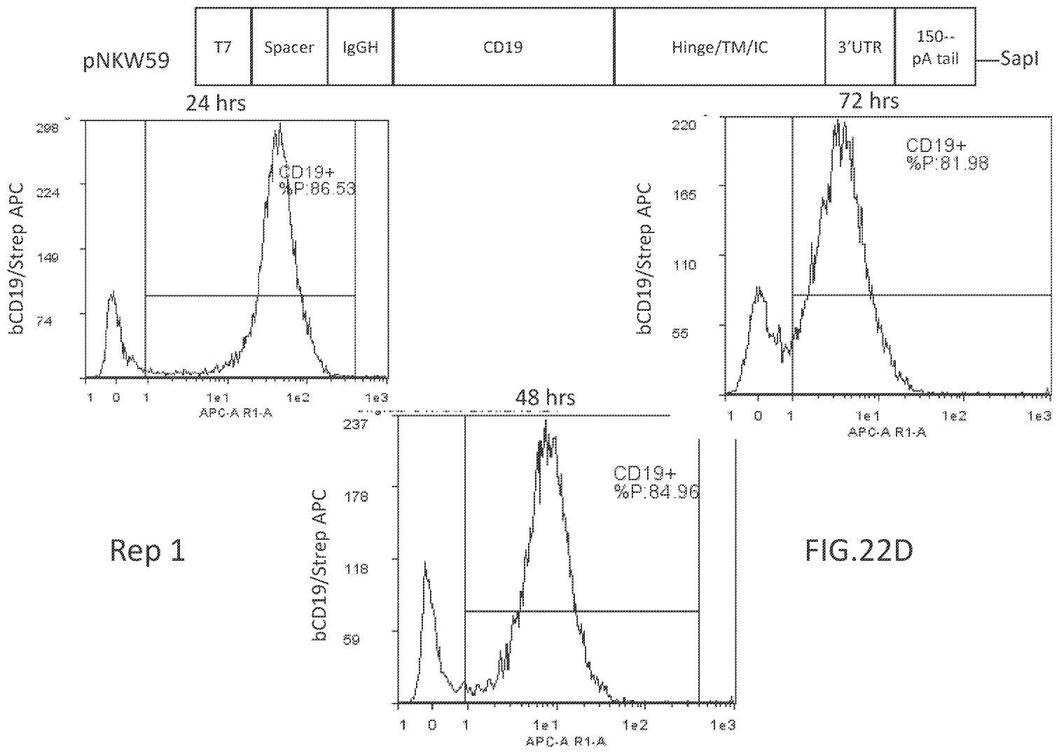

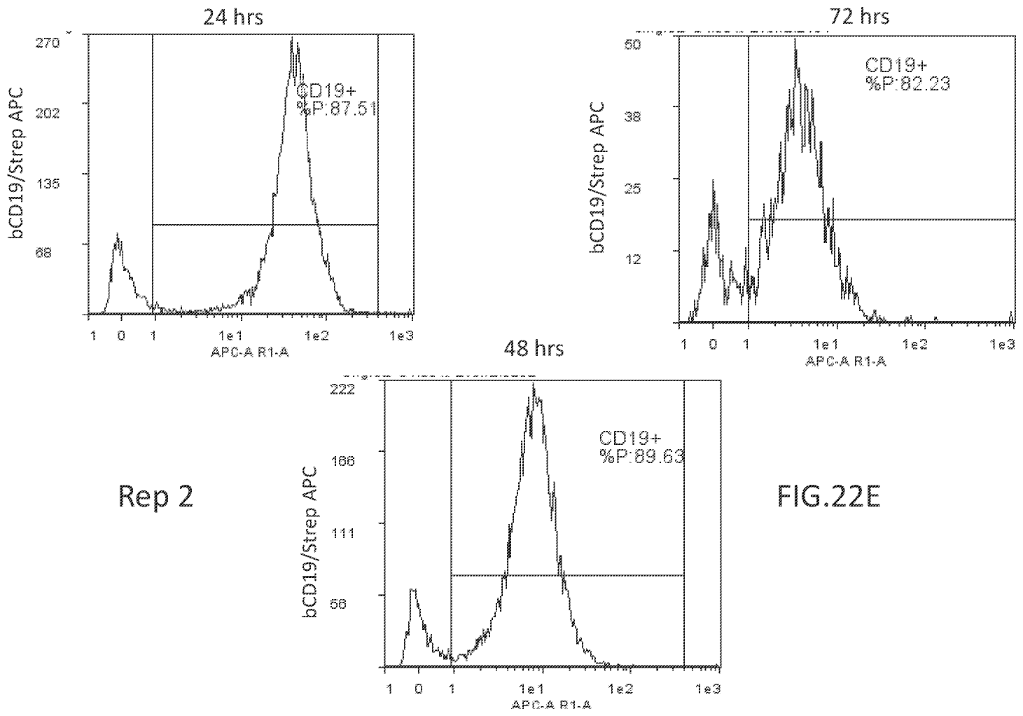

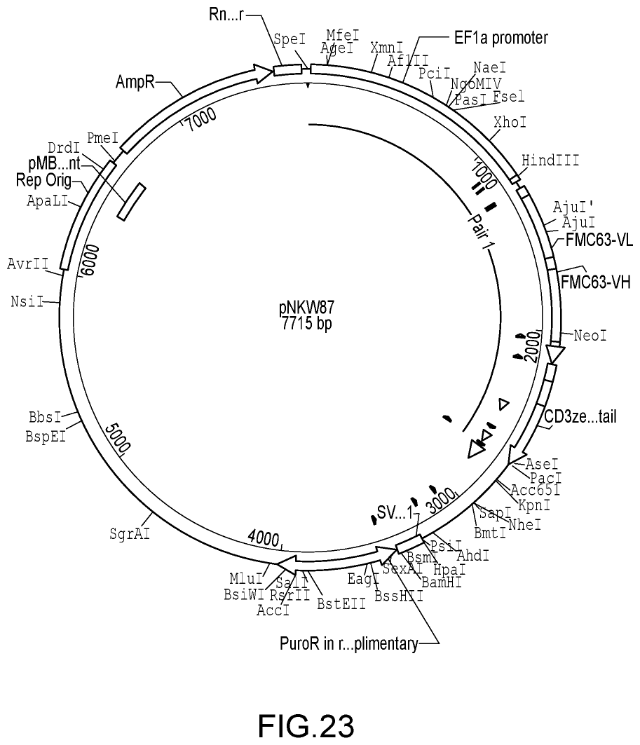

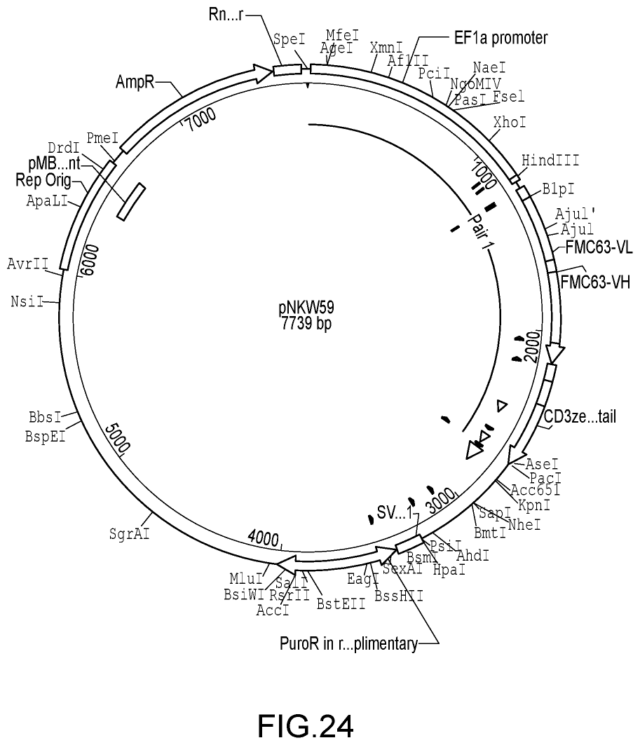

This example shows the rational for developing a mono-peptide CD19CAR. A Tri-peptide CD19 CAR that was previously used for making a stable CD19-CAR NK92 cell line was used to generate mRNA for electroporation of PB-NK cells. After overnight recovery from electroporation, CD19 CAR was detected using flow cytometry and a biotinylated anti CAR (F(ab′)2) followed by streptavidin-APC. As a control for the specificity of the detection reagent, a streptavidin-APC only staining was used for each sample. As shown in FIG. 15A, the no transfection negative control did not express the CD19 CAR, when the positive control cell line showed high expression of the CD19 CAR. To evaluate the electroporation protocol, two different RNA templates (GFP and PDL1) were used and they both showed high expression of the corresponding encoded protein 24 hours post electroporation (FIG. 15B). The Tri-peptide CD 19 CAR mRNA could not be expressed in PB-NK cells (FIG. 15C). To ensure that lack of CD19 CAR expression is not PB-NK cell specific, memory T cells (Tscm) were electroporated with the same CD19 CAR mRNA using three different protocols with increasing electric pulses. Neither the un-transfected negative control (FIG. 16A) nor the transfected Tscm cells (FIGS. 16B-D) express the CD19 CAR. The next step was to design a mono-peptide CD19 CAR with the domains optimized for mRNA expression in primary NK cells. Two CAR molecules (differing in their signal peptide) targeting the CD19 antigen were designed as shown in FIG. 17. The CAR DNA was linearized with SapI and served as a template for in vitro transcription of mRNA. Next, PB-NK cells were electroporated with the CAR mRNA. After overnight recovery, CD19 CAR was detected using flow cytometry and a biotinylated CD19 followed by streptavidin-APC. As a control for the specificity of the detection reagent, a streptavidin-APC only staining was used for each sample. As shown in FIGS. 18A-18B, the no transfection control does not express the CD19 CAR. On the other hand, PB-NK cells transfected with both constructs (FIGS. 18C, 18D and 18E, 18F) showed high levels of CD19 CAR. The CAR molecules showed cytotoxic activity against a SUP-B15 cell line expressing the CD19 antigen (parental) and no activity against the CD19-negative variant of the same cell line (FIG. 19A, and B). Use of memory NK cells (CIML) is promising for prolonging survival and activity of NK cells in a tumor microenvironment. PB-NK cells were subjected to cytokine treatment (IL12/IL18/N-803) for 16 hours. Cytokines were removed and both control and CIML cells were electroporated with pNKW87 mRNA. As shown in FIGS. 20A-20B, the transfected cells show cytotoxic activity only on the CD19-positive target cells in both memory (FIG. 20A) and control (FIG. 20B) PBNK cells. The mono-peptide CD19 CAR was next tested in activated T cells (control for Tscm) and Tscm cells. For this experiment, stability of CD19 CAR expression was also tested over a course of 72 hours. As shown in FIGS. 21B and 21C (pNKW87) and FIGS. 21D and 21E (pNKW59), the CD19 CAR levels stay high even 72 hours post-electroporation of activated T cells. The no-transfection negative control (FIG. 21A), shows no CD19 CAR expression. Similar stability studies were done with electroporation of Tscm cells with the same CAR RNA molecules (FIGS. 22B, and 22C and FIGS. 22D and 22E: pNKW87 and pNKW59 respectively). The results show high stability of CD19 CAR mRNA in transfected Tscm cells even 72 hours post-transfection. The no-transfection negative control (FIG. 22A), shows no CD19 CAR expression. The vectors encoding the CD19 CAR are shown in FIGS. 23 and 24.

### Example 6

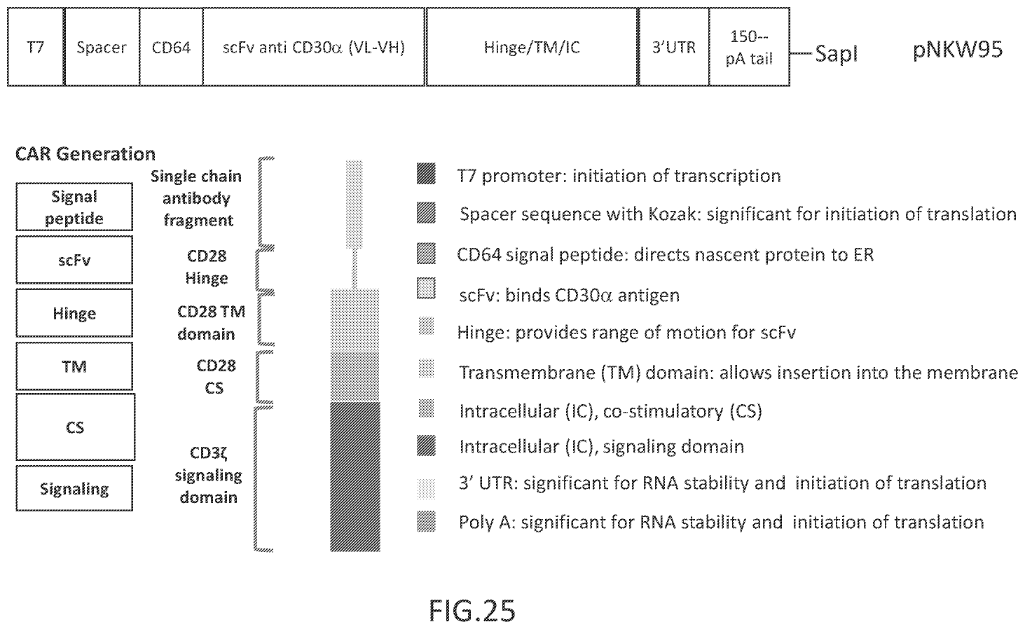

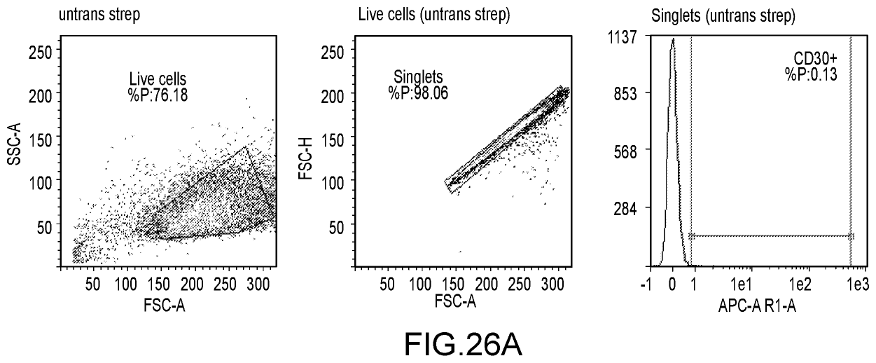

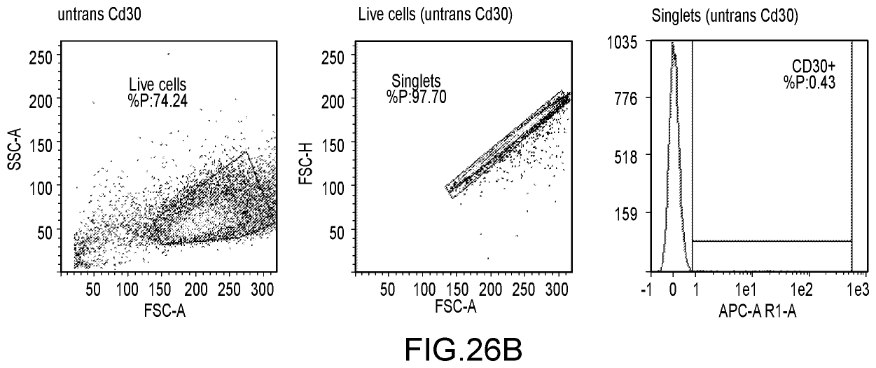

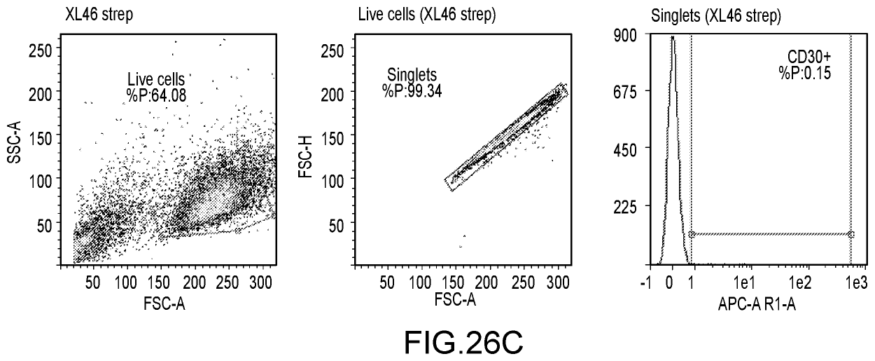

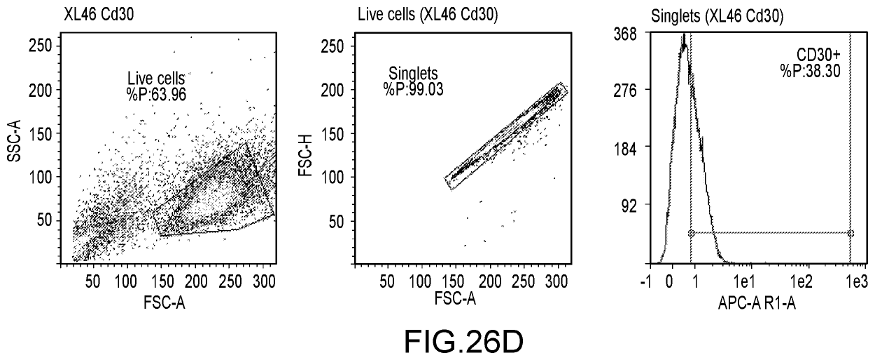

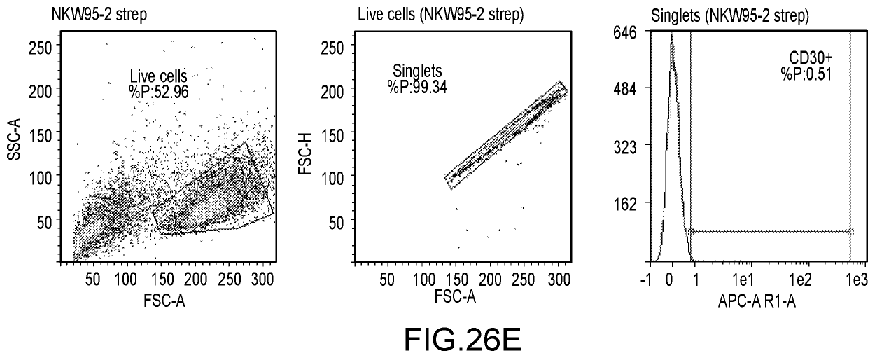

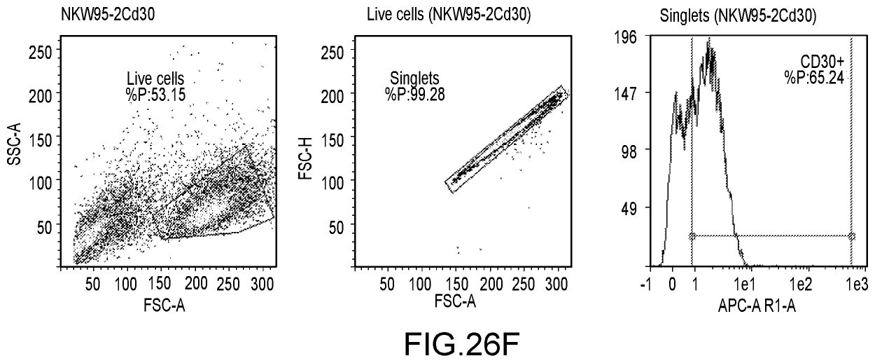

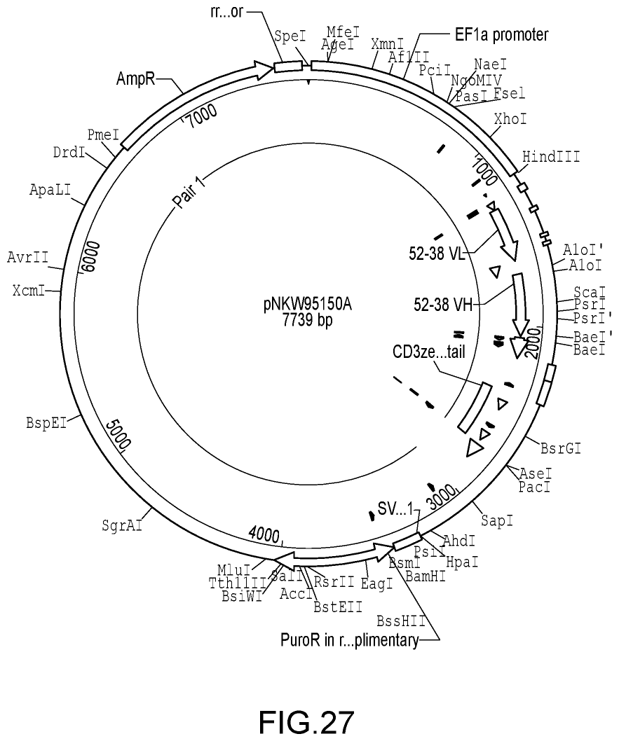

This example shows the results with CD30 pNKW95 (FIGS. 25, 26A-26F and -27). A CAR molecule targeting the CD30α antigen was designed in a vector containing the hinge, TM and co-stimulatory domains of CD28 as well a CD3ζ signaling domain and a 150p-A (FIG. 25). The vector was digested with SapI and the linearized DNA was used as a template for mRNA generation. CENK cells were electroporated with the pNKW95 mRNA. After overnight recovery, CD30 expression was detected using flow cytometry and a biotinylated CD30 followed by streptavidin-APC. A streptavidin-APC staining alone was used to confirm specificity of the detection method. A no-transfection control (FIGS. 26A, 26B) shows no endogenous expression of CD30 in CENK cells. The original plasmid (pXL46) from which the pNKW95 was derived was used for comparison. The pXL46 does not have a long poly A tail, so a poly A tail (less than 50 nucleotide) was added during in vitro transcription. As shown in FIGS. 26E, 26F, the pNK95 shows higher CD30 CAR expression compared to the original construct (pXL46: FIG. 26C, 26D). The vector encoding the CD30 CAR is shown in FIG. 27.

1. A chimeric antigen receptor (CAR) comprising:
a T7 promoter, a spacer sequence, a signal peptide, an antigen binding domain, a hinge region, a transmembrane (TM) domain, and an intracellular domain;
wherein the signal peptide comprises a cluster of differentiation 64 (CD64) and/or an IgG heavy chain variable gene (IgGHv) signal peptide; and
wherein the antigen binding domain binds to an antigen selected from the group consisting of cluster of differentiation 19 (CD19), B-cell maturation antigen (BCMA), B7 homolog 4 (B7H4), SARS-CoV-2 spike, and cluster of differentiation 30 alpha (CD30α).

2. The CAR of claim 1, wherein the signal peptide comprises SEQ ID NO: 1 or SEQ ID NO:2.

3. The CAR of claim 1, wherein the antigen binding domain that binds to CD19 comprises at least 90%, at least 95%, or up to 100% sequence identity to SEQ ID NO:4.

4. The CAR of claim 1, wherein the antigen binding domain that binds to BCMA comprises at least 90%, at least 95%, or up to 100% sequence identity to SEQ ID NO:5 or SEQ ID NO:6.

5. The CAR of claim 1, wherein the antigen binding domain that binds to B7H4 comprises at least 90%, at least 95%, or up to 100% sequence identity to SEQ ID NO:7.

6. The CAR of claim 1, wherein the antigen binding domain that binds to SARS-CoV-2 spike comprises at least 90%, at least 95%, or up to 100% sequence identity to SEQ ID NO:8.

7. The CAR of claim 1, wherein the antigen binding domain that binds to CD30α comprises at least 90%, at least 95%, or up to 100% sequence identity to SEQ ID NO:57.

8. The CAR of claim 1, wherein the hinge region is a cluster of differentiation 28 (CD28) hinge region having SEQ ID NO:9.

9. The CAR of claim 1, wherein the TM domain is a CD28 TM domain having SEQ ID NO:10.

10. The CAR of claim 1, wherein the intracellular domain comprises a co-stimulatory domain comprises a CD28 cytoplasmic domain having SEQ ID NO:11.

11. The CAR of claim 1, wherein the intracellular signaling domain comprises a cluster of differentiation 3 zeta (CD3ζ) cytoplasmic domain having SEQ ID NO:12.

12. The CAR of claim 1, wherein the CAR comprises an amino acid sequence having at least 90%, at least 95%, or up to 100% sequence identity to an amino acid sequence selected from the group consisting of SEQ ID NO:14, SEQ ID NO: 15, SEQ ID NO: 16, SEQ ID NO: 17, SEQ ID NO:18, SEQ ID NO:19, SEQ ID NO: 20, SEQ ID NO:21, SEQ ID NO:22 and SEQ ID NO:58.

13. A nucleic acid construct encoding the CAR of claim 1, wherein the nucleic acid construct comprises a nucleic acid sequence selected from the group consisting of SEQ ID NO:24, SEQ ID NO:25, SEQ ID NO:26, SEQ ID NO: 27, SEQ ID NO:28, SEQ ID NO:29, SEQ ID NO:30, SEQ ID NO:31 SEQ ID NO: 32 and SEQ ID NO:59.

14. (canceled)

15. An expression vector encoding the CAR of claim 1.

16. The expression vector of claim 15 having a nucleic acid sequence selected from the group consisting of SEQ ID NO:33, SEQ ID NO: 34, SEQ ID NO: 35, SEQ ID NO:36, SEQ ID NO:37, SEQ ID NO:38, SEQ ID NO: 39, SEQ ID NO: 40, SEQ ID NO:41, and SEQ ID NO:42.

17. A modified primary Natural Kill (NK) cell modified with an RNA molecule comprising one or more nucleic acids of a T7 promoter, a spacer sequence, a signal peptide sequence portion, an antigen binding domain sequence portion, a hinge region sequence portion, a transmembrane (TM) domain sequence portion and an intracellular domain sequence portion;
wherein the signal peptide sequence comprises a sequence encoding cluster of differentiation 64 (CD64) and/or an IgG heavy chain variable gene (IgGHv);
wherein the antigen binding domain comprises a sequence encoding an antigen binding portion that binds to an antigen selected from the group consisting of cluster of differentiation 19 (CD19), B-cell maturation antigen (BCMA), B7 homolog 4 (B7H4), SARS-CoV-2 spike, and cluster of differentiation 30 alpha (CD30α); and
wherein the nucleic acid sequences are operably linked to each other as a single polynucleotide.

18. The modified primary NK cell of claim 17, wherein the intracellular domain sequence portion comprises a CD28 cytoplasmic domain having SEQ ID NO: 11 and/or a cluster of differentiation 3 zeta (CD3ζ) cytoplasmic domain having SEQ ID NO: 12.

19. (canceled)

20. (canceled)

21. A method for generating modified primary CAR-NK cells comprising transfecting a primary NK cell with a recombinant nucleic acid construct of claim 13.

22. A method of immunotherapy for treating cancer in a subject in need thereof, the method comprising administering to the subject a therapeutically effective amount of a pharmaceutical composition comprising a genetically modified NK cell of claim 17.

23. (canceled)

24. (canceled)

25. (canceled)

26. (canceled)

27. A pharmaceutical composition comprising a modified NK cell of claim 17 and a pharmaceutically acceptable carrier.
